(19) United States
(12) Patent Application Publication
(10) Pub. No.: US 2024/0325567 A1
(43) Pub. Date: Oct. 3, 2024

(54) CELL THERAPY

(71) Applicant: IMPERIAL COLLEGE INNOVATIONS LIMITED, London (GB)

(72) Inventors: Uta GRIESENBACH, London (GB); Eric ALTON, London (GB); Nora Katherine CLARKE, London (GB)

(21) Appl. No.: 18/555,403

(22) PCT Filed: Apr. 13, 2022
(86) PCT No.: PCT/GB2022/050933

§ 371 Date: Oct. 13, 2023

(30) Foreign Application Priority Data
Apr. 13, 2021 (GB) 2105278.2

(51) Int. Cl. A61K 48/00; C12N 5/0786; C12N 15/86

(52) U.S. Cl. CPC A61K 48/0033; C12N 5/0645; C12N 15/86; C12N 2506/13; C12N 2510/00; C12N 2740/15043

(57) ABSTRACT

The present invention relates to methods for the production of immune cells and immunomodulatory cells expressing a transgene of interest, using lentiviral vectors. The invention also relates to populations of cells produced by such methods and uses thereof.

### FIELD OF THE INVENTION

The present invention relates to methods for the production of modified immune cells, immunomodulatory cells, induced pluripotent stem cells (iPSCs) or iPSC-derived cells expressing a transgene of interest, using lentiviral vectors. The invention also relates to populations of cells produced by such methods and uses thereof.

### BACKGROUND TO THE INVENTION

There have been major efforts to develop gene therapy approaches for genetic and acquired lung diseases, using in vivo and ex vivo approaches. Many of these diseases lack effective or feasible treatment options and gene therapy can offer a long term solution, particularly in the case of genetic disorders. In vivo approaches to the lung have been popular, as initially it was believed the lung would be an easy organ to target due to the relative ease of access. However the lung has many barriers to airborne pathogens which have proven effective at limiting both viral and non-viral vector-access to the lung epithelium.

Further, many diseases with severe pulmonary manifestations are not limited to the lung epithelium, or are in part due to a lack or absence of a secreted protein in the lung milieu, such as is the case in pulmonary alveolar proteinosis (PAP) and α1-antitrypsin deficiency. Therefore, gene therapy targeting delivery of these therapeutic secreted proteins to the lung epithelium may not be sufficient to provide a therapeutic effect, even if under normal or healthy conditions these lung epithelial cells secrete the protein.

Ex vivo cell transduction to produce cells secreting therapeutic proteins, and administered the secretory cells to patients has the potential to be an efficient alternative to in vivo gene transfer and negate difficulties associated with delivery and entry of a vector directly to the lung. In particular, macrophages are a prime candidate as a vehicle with which to deliver sustained transgenic protein to the lung, because sources of autologous macrophages are relatively accessible in patients (peripheral blood mononuclear cells), and studies have shown that transplanted macrophages resemble alveolar macrophages in their long-half life and persistence in lungs.

However, targeting macrophages for genetic modification has proven problematic in practice. Macrophages are professional phagocytes and so are naturally equipped to degrade nucleic acids and foreign materials, and produce anti-viral responses. Non-viral approaches to gene modification of these cells have shown little to no success. As such, there remains an unmet need for a successful vector that can efficiently transduce macrophages and provide long lasting gene expression in these cells.

It is an object of the invention to address one or more of these problems. In particular, it is an object of the invention to provide a method for modifying modified immune cells, immunomodulatory cells, iPSCs or iPSC-derived cells, and particularly macrophages, using lentiviral vectors, wherein the modified modified immune cells, immunomodulatory cells, iPSCs or iPSC-derived cells express a transgene of interest.

### SUMMARY OF THE INVENTION

The present inventors have previously developed a lentiviral vector, which has been pseudotyped with hemagglutinin-neuraminidase (HN) and fusion (F) proteins from a respiratory paramyxovirus, comprising a promoter and a transgene. Typically, the backbone of the vector is from a simian immunodeficiency virus (SIV), such as SIV1 or African green monkey SIV (SIV-AGM). The HN and F proteins function, respectively, to attach to sialic acids and mediate cell fusion for vector entry to target cells. The present inventors discovered that this specifically F/HN-pseudotyped lentiviral vector can efficiently transduce airway epithelial cells. However, the ability of this lentiviral vector to transduce other cell types had not been investigated.

The present inventors have now shown for the first time that an F/HN-pseudotyped lentiviral vector can efficiently transduce immune cells. In particular, the inventors have shown for the first time that bone marrow derived macrophages, and monocytes from bone marrow prior to differentiation to a macrophage state can be transduced using an F/HN pseudotyped lentivirus to express both secreted and non-secreted transgenes in a dose-dependent manner. The inventors have also shown that modified macrophages produced in this way express significant levels of secreted transgene in vivo when administered to mice. Thus, the present inventors have demonstrated for the first time the therapeutic potential for an ex vivo cell therapy approach, particularly a macrophage-based ex vivo cell therapy approach, to expressing therapeutic proteins in the lung, utilising an F/HN pseudotyped lentivirus.

Accordingly, the invention provides an ex vivo method for obtaining immune cells, immunomodulatory cells, induced pluripotent stem cells (iPSCs) or iPSC-derived cells modified to express a transgene of interest, said method comprising transducing the cells with a lentiviral vector comprising the transgene, wherein the transgene is a secreted therapeutic protein. The lentiviral vector may be pseudotyped with haemagglutinin-neuraminidase (HN) and fusion (F) proteins from a respiratory paramyxovirus or G glycoprotein from Vesicular Stomatitis Virus (G-VSV). The secreted therapeutic protein may be selected from Alpha-1 Antitrypsin (A1A1), Factor VIII, Surfactant Protein B (SFTPB), Factor VII, Factor IX, Factor X, Factor XI, van Willebrand Factor, Granulocyte-Macrophage Colony-Stimulating Factor (GM-CSF), an anti-inflammatory protein (e.g. IL-10 or TGFβ) or monoclonal antibody, or a monoclonal antibody against an infectious agent.

The invention further provides an ex vivo method for obtaining immune cells, immunomodulatory cells, iPSC, or iPSC-derived cells modified to express a transgene of interest, said method comprising transducing the cells with a lentiviral vector comprising the transgene, wherein the lentiviral vector is pseudotyped with haemagglutinin-neuraminidase (HN) and fusion (F) proteins from a respiratory paramyxovirus or G glycoprotein from Vesicular Stomatitis Virus (G-VSV). The transgene may encode a non-secreted protein involved in macrophage biology, optionally selected from CFTR, CSF2RA, CSF2RB or TRIM-72.

In an ex vivo method of the invention, the lentiviral vector may be selected from the group consisting of a Human immunodeficiency virus (HIV) vector, a Simian immunodeficiency virus (SIV) vector, a Feline immunodeficiency virus (FIV) vector, an Equine infectious anaemia virus (EIAV) vector, and a Visna/maedi virus vector. Preferably the lentiviral vector may be a SIV vector. The respiratory paramyxovirus may be a Sendai virus. The vector may further comprise a promoter, which is optionally selected from the group consisting of a hybrid human cytomegalovirus (CMV) enhancer/elongation factor 1 a (EF1 a) promoter (hCEF), a CMV promoter, an EF1 a promoter, and a steroid-regulated promoter, preferably a hCEF promoter. The immune cells, immunomodulatory cells, iPSC, or iPSC-derived cells may be derived from peripheral blood, cord blood, bone marrow, fibroblasts or adipose tissue. The immune cells, immunomodulatory cells, iPSC, or iPSC-derived cells may be: (a) immune cells which are peripheral blood mononuclear cells (PBMCs), optionally macrophages or monocytes; (b) immunomodulatory cells, optionally mesenchymal stem cells (MSCs); or (c) iPSCs or iPSC-derived cells. The immune cells, immunomodulatory cells, iPSC, or iPSC-derived cells may be: (a) differentiated macrophages, optionally differentiated from monocytes, iPSCs or MSCs prior to transduction; (b) monocytes, iPSCs or MSCs, and which are differentiated to macrophages after the cells have been transduced with the lentiviral vector; or (c) iPSC-derived cells, optionally iPSC-derived epithelial cells, which are differentiated from iPSC prior to transduction with the lentiviral vector. The immune cells, immunomodulatory cells, iPSC, or iPSC-derived cells may (i) be naïve; or (ii) have previously been mobilised.

An ex vivo method of the invention may further comprise: (a) a step of expanding the immune cells, immunomodulatory cells, iPSC, or iPSC-derived cells prior to transduction with the lentiviral vector; or (b) a step of expanding the immune cells, immunomodulatory cells, iPSC, or iPSC-derived cells after transduction with the lentiviral vector.

An ex vivo method of the invention may further comprise one or more step to isolate and/or concentrate the immune cells, immunomodulatory cells, iPSC, or iPSC-derived cells, wherein optionally said one or more step is carried out: (a) prior to or after the immune cells, immunomodulatory cells, iPSC, or iPSC-derived cells are transduced with the lentiviral vector; and/or (b) prior to or after a step of expanding the immune cells, immunomodulatory cells, iPSC, or iPSC-derived cells.

The invention further provides a population of modified immune cells, immunomodulatory cells, iPSC, or iPSC-derived cells comprising a transgene of interest, which immune cells, immunomodulatory cells, iPSC, or iPSC-derived cells are obtainable by a method of the invention.

The invention also provides a population of modified immune cells, immunomodulatory cells, iPSC, or iPSC-derived cells comprising a transgene of interest obtainable by an ex vivo method of the invention for use in a method of gene therapy. Said method of gene therapy may comprise carrying out an ex vivo method of the invention and administering the modified cells produced by said method to a patient. The immune cells, immunomodulatory cells, iPSCs or iPSC-derived cells may be: (a) autologous cells derived from a patient to be treated; or (b) allogenic cells derived from an individual other than the patient. Said gene therapy may be for the treatment of Cystic Fibrosis (CF); Alpha 1-antitrypsin Deficiency (A1AD); Pulmonary Alveolar Proteinosis (PAP); Chronic obstructive pulmonary disease (COPD); a surfactant deficiency; an inflammatory or allergic lung condition; an infection of the lung; lung cancer; asthma or a fibrotic lung condition.

The invention further provides a lentiviral vector as defined herein for use in a method of gene therapy, wherein said method comprises the steps of: (a) transducing immune cells, immunomodulatory cells, iPSCs or iPSC-derived cells with the lentiviral vector to produce modified immune cells, immunomodulatory cells, iPSCs or iPSC-derived cells expressing a transgene of interest; and (b) administering the resulting modified immune cells, immunomodulatory cells, iPSCs or iPSC-derived cells to a patient. Said method of gene therapy may comprise carrying out an ex vivo method of the invention and administering the modified immune cells, immunomodulatory cells, iPSC, or iPSC-derived cells produced by said method to a patient.

The invention further provides a composition comprising a population of modified immune cells, immunomodulatory cells, iPSCs or iPSC-derived cells comprising a transgene of interest obtainable by an ex vivo method of the invention, and optionally a pharmaceutically acceptable excipient, buffer or diluent.

The invention provides the use of a population of modified immune cells, immunomodulatory cells, iPSCs or iPSC-derived cells obtainable by an ex vivo method of the invention in the manufacture of a medicament for use in a method of gene therapy.

The invention also provides a gene therapy method comprising administering to a subject in need thereof a therapeutically effective amount of a population of modified immune cells, immunomodulatory cells, iPSCs or iPSC-derived cells comprising a transgene of interest, which cells are obtainable by an ex vivo method of the invention.

### BRIEF DESCRIPTION OF THE DRAWINGS

FIG. 1: Bone marrow derived macrophages (BMDM) express EGFP when transduced with lentivirus. Graphs showing the percentage of EGFP-positive BMDM from C57BL/6 mice following transduction with SIV-F/HN expressing an EGFP transgene at increasing multiplicities of infection (MOI). a) Cells transduced during the differentiation process, or b) post-differentiation (n=6/MOI). Bars represent median. **p<0.01, ***p<0.001 Kruskal Wallis test with Dunn's multiple comparisons.

FIG. 2: Bone marrow derived macrophages (BMDM) secrete GM-CSF when transduced with lentivirus. Graphs showing the concentration of GM-CSF produced by BMDM from C57BL/6 mice following transduction with SIV-F/HN expressing a GM-CSF transgene at increasing multiplicities of infection (MOI). a) Cells transduced during the differentiation process, or b) post-differentiation (n=6/MOI). Bars represent median. *p<0.05, **p<0.01, ***p<0.001 Kruskal Wallis test with Dunn's multiple comparisons.

FIG. 3: F/HN lentivirus has a maximum feasible dose of MOI 50 in bone marrow derived macrophages (BMDM). BMDM were transduced with F/HN pseudotyped simian immunodeficiency virus (SIV) expressing a) GFP or b) GM-CSF transgene at multiplicities of infection (MOI) of 20 or 50. (n=6/MOI) and then either: a) harvested on day 14 and the percentage of EGFP positive macrophages was determined, or b) on day 16, GM-CSF levels were measured. Bars represent median. *p<0.05, **p<0.01, ***p<0.001 Kruskal Wallis test with Dunn's multiple comparisons.

FIG. 4: Bone marrow derived macrophages (BMDM) are unsuccessfully transfected using the reagent PEI PRO. BMDM were transfected with a plasmid expressing gaussia luciferase under the control of the hCEF promoter in a non-viral expression cassette. Bone marrow was differentiated into macrophages by supplying M-CSF (100 ng/ml) in culture and cells were transfected on day 7 (n=6/MOI). 48 hours post transfection, all media (1 ml per well) was removed and analysed using the Pierce™ Gaussia Luciferase Glow Assay Kit from Thermo Fischer Scientific. Media from A549 cells transfected under the same protocol are shown in comparison. Bars represent median. **p<0.01 Mann-Whitney t-test, all significant interactions are shown.

FIG. 5: Bone marrow derived macrophages (BMDM) transduced with F/HN lentivirus secrete detectable levels of GM-CSF in the mouse lung. a) BMDM were transduced post-differentiation with F/HN lentivirus expressing GM-CSF then delivered to the mouse lung using. Graph shows significant GM-CSF in the BALF of mice treated with transduced BMDM.

FIG. 6: Bone marrow derived macrophages (BMDM) transduced with F/HN lentivirus secrete detectable levels of Gaussia luciferase (Gluc) in the mouse lung in a dose dependent manner. BMDM were transduced post-differentiation with F/HN lentivirus expressing Gluc then delivered to the mouse lung. Graphs show dose-dependent Gluc secretion in tissue homogenate (A) and the BALF (B) of mice treated with transduced BMDM at 1 week time point. Bars represent median value.

FIG. 7: Bone marrow derived macrophages (BMDM) transduced with F/HN lentivirus remain in the mouse lung and secrete detectable levels of Gaussia luciferase (Gluc) for up to two weeks. BMDM were transduced post-differentiation with F/HN lentivirus expressing Gluc then delivered to the mouse lung. (A) Gluc levels in lung homogenate. (B) Gluc levels in the BALF. (C) Number of transplanted cells in the BALF. Bars represent median value. *p<0.05, **p<0.01, Kruskal Wallis test with Dunn's multiple comparisons, stars above data sets indicate significant difference to untransduced controls. All significant interactions are shown.

FIG. 8: Bone marrow derived macrophages (BMDM) transduced with VSV-G lentivirus express a GFP transgene at promoter-specific levels of intensity. Graphs show a) percentage of EGFP positive macrophages and b) mean fluorescence intensity from BMDM transduced with VSV-G pseudotyped SIV expressing an EGFP transgene under the control of either a CMV, hCEF, or EF1a promoter. Bars represent median value. ****p<0.0001, Ordinary One-Way ANOVA with Holm-Sidak's multiple comparisons test comparing promoter data sets (exl UT). All significant interactions are shown.

FIG. 9: Bone marrow derived macrophages (BMDM) transduced with F/HN lentivirus to secrete GM-CSF in the mouse lung, lead to robust changes in phenotypic markers of disease in mouse models of pulmonary alveolar proteinosis (PAP). Graphs show a) turbidity (optical density at 600 nm) of BALF collected 2 weeks or 4 weeks post-delivery, and b) concentration of surfactant protein D (SP-D) in BALF. Bars represent median value. Two-way ANOVA with Sidak's multiple comparisons test, all significant interactions are indicated.

FIG. 10: Sustained Glux expression in vivo following bone marrow derived macrophages (BMDMs) transplantation. BMDMs were transduced ex vivo with rSIV.F/HN lentivirus encoding murine Glux under the control of the hCEF promoter at MOI 20. Following a single dose of 3×10⁶ cells delivered via oropharyngeal delivery Glux expression was detected in (a) bronchoalveolar lavage fluid (BALF) and (b) lung homogenate (LH) 2, 4, 8, and 16 weeks later (n=6/group). Glux expression was compared to samples from control mice which received a corresponding dose of un-transduced macrophages and were harvested at week 2 (n=6), maximum reported Glux expression in these mice is indicated by red dotted line. Glux was quantified in 10 μl of BALF and lung homogenate, the latter were then normalised to total protein concentration (mg/ml). Median values plotted; range indicated by error bars. (*=p<0.05, **=p<0.01, ***=p<0.001, ****=p<0.0001, Kruskal-Wallis, Dunn's multiple comparisons).

FIG. 11: Retention of bone marrow derived macrophages (BMDMs) in the lung following transplantation. A single dose of 3×106 BMDMs transduced ex vivo with rSIV.F/HN.hCEF.Glux were delivered via oropharyngeal delivery to WT mice. (a) The number of donor-derived cells (CD45.1⁺) in BALF samples were identified by flow cytometry in the BALF of mice harvested 2, 4, 8, and 16 weeks post-delivery (n=6/group). Absolute counts of donor-derived cells in BALF were extrapolated using counting beads. **=p<0.01, Kruskal-Wallis, Dunn's multiple comparisons. Each circle represents a single mouse, bars indicate median.

FIG. 12: Relative transduction efficiency and integration of VSV-G and F/HN pseudotyped rSIV lentivirus in bone marrow-derived macrophages (BMDMs). Comparisons between rSIV vectors with a VSV-G and F/HN pseudotype in BMDMs transduced at MOI 46. Untransduced cells served as control samples. Transduction efficiency was calculated by (a) flow cytometry analysis of EGFP transgene expression (n=6), and (b) a ddPCR encapsulation assay quantifying cells positive for viral cDNA (n=5). (c) Vector copy number (VCN) ddPCR analysis of reverse transcribed lentiviral genomes was performed from bulk extracted DNA samples (n=6), and then (d) corrected for transduction efficiency as quantified by the encapsulation assay. BMDMs were transduced for 24 hours and then analysed on day 7. Each sample was split into thirds, ⅓ was then analysed by flow cytometry, another ⅓ by encapsulation. The final ⅓ was divided equally into two and used to procure RNA and DNA extractions. Flow cytometry capture by BD Accuri™ C6 Plus Flow Cytometer, analysis in FlowJo software. Each data point represents an individually transduced well of BMDMs. Bars represent median. (a) Mann-Whitney t-test, (b-d) Kruskall-Wallis, Dunn's multiple comparisons, ns=not significant.

FIG. 13: Transgene mRNA and vector specific RNA comparisons in BMDMs transduced with VSV-G and F/HN pseudotyped rSIV lentivirus. The concentration of RRE (left) and WPRE (right) positive RNA copies were quantified by ddPCR in BMDMs transduced by rSIV vectors with a VSV-G or F/HN pseudotype (MOI 46). Untransduced cells served as control. RNA analysis performed on bulk extracted samples of RNA, where RRE is specific to viral genomes, and WPRE is present on both viral genomes and transgene mRNA. BMDMs were transduced for 24 hours and then analysed on day 7. Each sample was split into thirds, ⅓ was then analysed by flow cytometry, another ⅓ by encapsulation. The final ⅓ was divided equally into two and used to procure RNA and DNA extractions. Bars represent median. ***=p<0.001, ****=p<0.0001, 2-wayANOVA, Sidak's multiple comparisons)

FIG. 14: Relative transduction efficiency of rSIV vectors encoding CMV, hCEF, and EF1a promoters in bone marrow-derived macrophages (BMDMs). The relative transduction efficiency of BMDMs following transduction with rSIV.VSV-G vectors expressing an EGFP transgene under the control of the CMV (left data set for each MOI), EF1a (centre data set for each MOI), and hCEF (right data set for each MOI) promoters at increasing multiplicities of infection (MOI: 10, 20, 50) (n=6/MOI). Transduction efficiency was calculated as the percentage of EGFP positive cells in culture by flow cytometry 7 days post-initiation of transduction (48 hours). Capture by BD Accuri™ C6 Plus Flow Cytometer. Each data point represents an individually transduced well of BMDMs. Bars represent median. *=p<0.05, ***=p<0.001, 2-wayANOVA, Sidak's multiple comparisons.

FIG. 15: Relative gene expression of CMV, hCEF, and EF1a promoters in bone marrow-derived macrophages (BMDMs). BMDMs were transduced with (a) rSIV.VSV-G and (b) rSIV.F/HN vectors expressing an EGFP transgene under the control of the CMV (C), EF1a (E), and hCEF (h) promoters at increasing multiplicities of infection (MOI: 10, 20, 50) (n=6). Gene expression quantified as mean fluorescent intensity (MFI) of EGFP expression by flow cytometry across previous transduction experiments. Colours and symbols relate to different experiments, black square: experiment 5.3.4; red circle: experiment 5.3.5; blue triangle: experiment 5.3.6. Capture by BD Accuri™ C6 Plus Flow Cytometer. Each data point represents an individually transduced well of BMDMs. Bars represent median. All crude vectors produced in small batches, except the large scale purified rSIV.F/HN.hCEF vector in experiment 5.3.4 (hL). *=p<0.05, ****=p<0.0001, 2-wayANOVA, Sidak's multiple comparisons.

FIG. 16: Induction of Gaussia luciferase (Glux) transgene expression in bone marrow derived macrophages (BMDMs). BMDMs were transduced with one of two regulated expression lentiviral systems: (b) a dual (2) vector system or (c) a single vector system for 24 hours (+SIV) (MOI 10-controlled for Glux encoding vector). Gene expression was induced by a 24-hour incubation with 10 μM of mifepristone (+M), and Glux was quantified in cell culture media every 24 hours for 5 days, where at each time point cells were incubated with fresh media (1 ml). Expression in induced samples was compared to non-induced controls at each time point to determine the degree of induction for each vector system, and significant differences between these conditions is indicated at each time point in (b) and (c). ns=p>0.05, ***=p<0.001, ****=p<0.0001, 2-wayANOVA, Sidak's multiple comparisons). Positive controls were transduced with rSIV.F/HN.Glux whose expression steadily increased over time and untransduced cells served as negative controls, and (a) the results of all test conditions and controls were plotted on one graph (n=6/group). (d) Untransduced controls exhibited increasing levels of leaky expression over time, each data set was fit to a model of simple linear regression. (e) The induction ratio of each vector system was calculated as the median difference in gene expression between induced and non-induced controls. Glux was quantified as relative light units (RLU) in 10 μl of media by Pierce™ Gaussia Luciferase Glow Assay Kit and normalised by cell number at the end of the experiment by quantification of total protein (mg/ml). Data plotted as median±range.

FIG. 17: Induction of Gaussia luciferase (Glux) transgene expression in bone marrow derived macrophages (BMDMs). BMDMs were transduced with one of two regulated expression lentiviral systems: (b) a dual (2) vector system or (c) a single vector system for 48 hours (+SIV) (MOI 10—controlled for Glux encoding vector). Gene expression was induced by a 24-hour incubation with 10 μM of mifepristone (+M) on day 2 and day 6. Glux was quantified in cell culture media every 24 hours for up to 7 days post transduction, where at each time point cells were incubated with fresh media (1 ml). Expression in induced samples was compared to non-induced controls at each time point to determine the degree of induction for each vector system, and significant differences between these conditions are indicated at each time point in (b) and (c). *=p<0.05, **=p<0.01, ***=p<0.001, 2-wayANOVA, Sidak's multiple comparisons). Positive controls were transduced with rSIV.F/HN.Glux whose expression steadily increased over time and untransduced cells served as negative controls, and (a) the results of all test conditions and controls were plotted on one graph (n=6/group). (d) Untransduced controls exhibited increasing levels of leaky expression over time. (e) The induction ratio of each vector system was calculated as the median difference in gene expression between induced and non-induced controls. Glux was quantified as relative light units (RLU) in 10 μl of media by Pierce™ Gaussia Luciferase Glow Assay Kit and normalised by cell number at the end of the experiment by quantification of total protein (mg/ml). Data plotted as median±range.

FIG. 18: In vivo induction of Gaussia luciferase (Glux) transgene expression from lung transplanted bone marrow derived macrophages (BMDMs). Bone marrow cells were transduced during differentiation into macrophages (Day 0-2; 48 hours) with a VSV-G-hCEF-Glux constitutive expression vector (VSV-G) or a dual vector regulated expression lentiviral system (MOI 20-controlled for Glux encoding vector). 1E6 cells/mouse were transplanted via oropharyngeal delivery to the lungs of wild type mice as differentiated macrophages on day 7. Mice either received untransduced cells (UT, n=6), cells transduced with VSV-G (n=6), or the inducible vector system (IN, n=12). 7 days later, n=6 of IN mice (IN+) and UT controls received daily administration of 0.5 mg/kg of mifepristone by IP injection for four days. The remaining n=6 IN mice did not receive mifepristone (IN−) and represent test conditions where Glux expression was not induced. All mice were harvested 12 days after cell delivery: the day following the final administration of mifepristone. Glux was quantified as relative light units (RLU) by Pierce™ Gaussia Luciferase Glow Assay Kit in 15 μl of (a) BALF, (b) Serum and (c) lung homogenate, the latter were then normalised to total protein concentration (mg/mL). The fold difference in Glux expression between treatment groups is indicated above data sets on each graph. Data plotted as median±range.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of’ when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the terms “vector”, “lentiviral vector” and “lentiviral F/HN vector” are used interchangeably to mean a lentiviral vector pseudotyped with hemagglutinin-neuraminidase (HN) and fusion (F) proteins from a respiratory paramyxovirus, unless otherwise stated. All disclosure herein in relation to lentiviral vectors of the invention applies equally and without reservation to SIV vectors that are pseudotyped with hemagglutinin-neuraminidase (HN) and fusion (F) proteins from a respiratory paramyxovirus (also referred to herein as SIV F/HN or SIV-FHN).

As used herein, the terms “transduced” and “modified” are used interchangeably to describe immune cells, immunomodulatory cells, iPSCs or iPSC-derived cells which have been modified to express a transgene of interest. Typically the modification occurs through transduction of the immune cells, immunomodulatory cells, iPSCs or iPSC-derived cells.

As used herein, the terms “titre” and “yield” are used interchangeably to mean the amount of lentiviral (e.g. SIV) vector produced by a method of the invention. Titre is the primary benchmark characterising manufacturing efficiency, with higher titres generally indicating that more retroviral/lentiviral (e.g. SIV) vector is manufactured (e.g. using the same amount of reagents). Titre or yield may relate to the number of vector genomes that have integrated into the genome of a target cell (integration titre), which is a measure of “active” virus particles, i.e. the number of particles capable of transducing a cell. Transducing units (TU/mL also referred to as TTU/mL) is a biological readout of the number of host cells that get transduced under certain tissue culture/virus dilutions conditions, and is a measure of the number of “active” virus particles. The total number of (active+inactive) virus particles may also be determined using any appropriate means, such as by measuring either how much Gag is present in the test solution or how many copies of viral RNA are in the test solution. Assumptions are then made that a lentivirus particle contains either 2000 Gag molecules or 2 viral RNA molecules. Once total particle number and a transducing titre/TU have been measured, a particle: infectivity ratio calculated. Amino acids are referred to herein using the name of the amino acid, the three-letter abbreviation or the single letter abbreviation.

As used herein, the term “immune cell” is used to mean a cell of the immune system (either adaptive or innate). An immune cell may be myeloid or lymphoid in lineage. Preferably the invention relates to immune cells of the myeloid lineage. An immune cell of the invention may be a differentiated cell or a progenitor cell. An immune cell of the invention, particularly a progenitor immune cell, may be pluripotent, multipotent or oligopotent. Non-limiting examples of myeloid immune cells according to the invention include myeloid progenitor cells, granulocyte/macrophage progenitors, myeloblasts, monocytes, neutrophils, eosinophils, basophils, macrophages and dendritic cells. Non-limiting examples of lymphoid immune cells according to the present invention include common lymphoid progenitors, lymphoid progenitors, progenitor B cells, natural killer (NK) cells and T cells (including Treg cells, NK T cells, cytotoxic T cells and helper T cells (Th1 and Th2). Immune cells of the invention may be mononuclear cells from peripheral blood (PBMCs) or mononuclear cells from cord blood. PBMCs and cord blood mononuclear cells may encompass monocytes and macrophages. Preferably an immune cell of the invention is a monocyte or macrophage. Macrophages can be detected or identified using conventional techniques, including marker profiles. For example, macrophages are typically CD45⁺, CD11b⁺, F4/80⁺, CD64⁺, and CD24⁻.

As used herein, the term “immunomodulatory cell” is used to mean a cell which can interact with one or more type of immune cell, and which is capable of modulating an immune response, enabling immunosuppression and/or tolerance induction. Preferably an immunomodulatory cell of the invention is a mesenchymal stem cell (MSC), also referred to interchangeably as a mesenchymal stromal cell. MSCs are multipotent mesoderm-derived progenitor cells. MSCs are heterogeneous non-hematopoietic fibroblast-like cells that can differentiate into cells of multiple lineages, such as chondrocytes, osteoblasts, adipocytes, myoblasts, and others. Adult MSCs can, for example, be isolated from the stroma of the bone marrow, peripheral blood, cord blood (or other neonat-associated tissue) and adipose tissue. Markers for undifferentiated MSCs include nucleostemin (an intracellular protein) as well as cell surface markers such as receptors for Bone Morphogenetic Proteins (BMPR), Endoglin, Stem Cell Factor Receptor (SCF R), and STRO-1. Any disclosure herein in relation to immunomodulatory cells applies equally and without restriction to MSCs.

As used herein, the terms “induced pluripotent stem cell” and “iPSC” are a type of pluripotent stem cell artificially derived from a non-pluripotent cell, typically an adult somatic cell or a terminally differentiated cell. iPSCs are believed to be identical to natural pluripotent stem cells, such as embryonic stem cells in many respects, such as in terms of the expression of certain stem cell genes and proteins, chromatin methylation patterns, doubling time, embryoid body formation, teratoma formation, viable chimera formation, and potency and differentiability. iPSCs are commonly generated from fibroblasts, such as dermal fibroblasts, haematopoietic cells, such as haematopoietic progenitor cells (HPCs), myocytes, neurons, epidermal cells. HPCs can be isolated from bone marrow, mononuclear cells obtained from cord blood or peripheral blood. Mononuclear cells obtained from peripheral blood may be referred to as peripheral blood mononuclear cells (PBMCs). Methods for generating iPSC from a non-pluripotent cell typically involve the introduction of certain factors, referred to as reprogramming factors into the non-pluripotent cell. Methods for generating iPSC are known in the art and within the routine practice of one of ordinary skill in the art.

As used herein, the term “iPSC-derived cell” refers to a cell which has been produced by the differentiation of an iPSC. Various approaches may be used with the present invention to differentiate iPS cells into cell lineages including, but not limited to, immune cells (e.g. macrophages or monocytes), immunomodulatory cells (e.g. MSCs), epithelial cells, hematopoietic cells, myocytes (e.g., cardiomyocytes), neurons, fibroblasts and epidermal cells, and tissues or organs derived therefrom. Methods for the differentiation of iPS cells are known in the art and within the routine practice of one of ordinary skill in the art.

As used herein the term “population” refers to a group of immune, immunomodulatory, iPSC and/or immune-derived cells as described herein. The population may consist or consist essentially of a single cell type, or may be a mixed population of different cell types. By way of non-limiting example, a mixed population may comprise macrophages and monocytes, in any proportion.

The term “xeno-free (XF)” or “animal component-free (ACF)” or “animal free”, when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components, therefore excluding mouse feeder cells or Matrigel™. Matrigel™ is a solubilised basement membrane preparation extracted from the Engelbreth-Holm-Swarm (EHS) mouse sarcoma, a tumour rich in extracellular matrix proteins to include laminin (a major component), collagen IV, heparan sulfate proteoglycans, and entactin/nidogen.

The term “defined”, when used in relation to a medium, an extracellular matrix, or a culture condition, refers to a medium, an extracellular matrix, or a culture condition in which the nature and amounts of approximately all the components are known. A “chemically defined medium” refers to a medium in which the chemical nature of approximately all the ingredients and their amounts are known. These media are also called synthetic media.

A culture, matrix or medium are “essentially free” of certain reagents, such as signalling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium.

Unless otherwise indicated, any nucleic acid sequences are written left to right in 5′ to 3′ orientation; amino acid sequences are written left to right in amino to carboxy orientation, respectively.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN: 1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino Acid | Codons | Degenerate Codon |
|---|---|---|
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, or at least about 100%, or at least about 150%, or at least about 200%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cystic fibrosis (CF) or any other disease described herein). Preferably said healthy individual(s) is not on medication affecting CF and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

Immune Cells, Immunomodulatory Cells, iPSCs and iPSC-Derived Cells

Many lung diseases with a genetic origin are not limited to the lung epithelium and are associated with defects in the production of secreted proteins critical to lung function, such as pulmonary alveolar proteinosis (PAP) and α1-antitrypsin deficiency (A1AD). To develop an effective gene therapy for such conditions, it is not necessary to target the lung epithelium directly with a vector. Rather, any cell type capable of secreting proteins can be modified to secrete the therapeutic protein, acting as a therapeutic protein “factory”. This modification can be carried out ex vivo, negating the need to deliver the vector directly to the lungs. Adopting an ex vivo approach also has the potential to alleviate safety concerns around delivering vectors directly to patients, minimise environmental exposure via vector shedding and may be cheaper than in vivo approaches, as lower vector titres will be required for efficient gene transfer.

Furthermore, some lung diseases are known to be caused by defects in the production of critical proteins are associated with deficient protein production by alveolar macrophages. Hereditary PAP is associated with mutations in the colony stimulating factor 2 receptor alpha gene in alveolar macrophages and it has been postulated that cystic fibrosis (CF) is associated with genetic aberrations in the same cells. In particular, chronic bacterial colonisation is a hallmark of CF lung disease, and has been linked with a reduced phagocytosis and bacterial killing by CF alveolar macrophages. CF alveolar macrophages also exhibit a hyper response to infection with an increased pro-inflammatory cytokine secretion profile.

Thus, where diseases find their root in defective alveolar macrophages or where mutations in these cells contribute to disease phenotypes (such as in CF and hereditary PAP), an approach using gene therapy to deliver transgenic macrophages ex vivo has the potential to alleviate disease burdens. Therefore, there may be particular benefit to the production of modified macrophages (or other cell types, such as immune cells, immunomodulatory cells, iPSCs and/or iPSC-derived cells as described herein) for use in gene therapy to treat such diseases.

In particular, the present invention has potential therapeutic application in diseases associated with genetic defects in a particular cell type in the lung, such as (alveolar) macrophages; in diseases where a secreted protein is missing as a result of a genetic defect (e.g. in various surfactant deficiencies); and/or where a secreted protein has a potential beneficial therapeutic effect in diseases of multifactorial aetiology, wherein a transgene can be expressed in the lung and the resulting protein secreted into the circulation to reach target sites around the body.

Accordingly, the invention provides an immune cell, immunomodulatory cell, induced pluripotent stem cell (iPSC) and/or iPSC-derived cell, which has been modified to express a transgene of interest. Immune cells, immunomodulatory cells, iPSCs and iPSC-derived cells are defined herein.

The invention particularly relates to the modification of immune cells selected from macrophages and monocytes. Thus, the invention may relate to the modification of differentiated macrophages. The invention may relate to the modification of monocytes, which may then be differentiated into macrophages after the monocytes have been transduced with a lentiviral (e.g. SIV) vector of the invention. The invention may relate to the modification of monocytes, which are differentiated into macrophages after the monocytes have been transduced with a lentiviral (e.g. SIV) vector of the invention and prior to administration to a patient. Any appropriate means may be used to differentiate (modified or unmodified) monocytes into macrophages, examples of suitable agents and protocols are known in the art. By way of non-limiting example, macrophage colony-stimulating factor (M-CSF) may be used to stimulate the differentiation of monocytes into macrophages. By way of further non-limiting example, activation actors as exemplified by interferon-gamma (IFNγ) and lipopolysaccharide (LPS) may be used to activate macrophages. The invention may relate to the modification of monocytes, which are transduced with a lentiviral (e.g. SIV) vector of the invention and then administered to a patient without first being differentiated into macrophages.

The invention also relates to the modification of iPSCs. The invention may relate to the modification of iPSCs, which may then be differentiated into another cell type (e.g. macrophages or monocytes) after the iPSCs have been transduced with a lentiviral (e.g. SIV) vector of the invention. The invention may relate to the modification of iPSCs, which are differentiated into another cell type (e.g. macrophages or monocytes) after the iPSCs have been transduced with a lentiviral (e.g. SIV) vector of the invention and prior to administration to a patient. The invention may relate to the modification of iPSCs, which are transduced with a lentiviral (e.g. SIV) vector of the invention and then administered to a patient without first being differentiated into another cell type.

The invention also relates to the modification of iPSC-derived cells (e.g. iPSC-derived epithelial cells, macrophages and/or monocytes). The invention may relate to the modification of iPSC-derived cells (e.g. iPSC-derived epithelial cells, macrophages and/or monocytes), which are themselves capable of further differentiation, and which may then be differentiated into another cell type (e.g. epithelial cells, macrophages or monocytes) after the iPSCs-derived cells have been transduced with a lentiviral (e.g. SIV) vector of the invention. The invention may relate to the modification of iPSCs-derived cells, which are themselves differentiated into another cell type (e.g. epithelial cells, macrophages or monocytes) after the iPSCs have been transduced with a lentiviral (e.g. SIV) vector of the invention and prior to administration to a patient. The invention may relate to the modification of iPSC-derived cells (e.g. iPSC-derived epithelial cells and/or macrophages), which are themselves already terminally differentiated prior to transduction with a lentiviral (e.g. SIV) vector of the invention and/or are administered to a patient without first being further differentiated into another cell type.

The invention may relate to the modification of progenitor cells, such as MSCs or iPSCs if there are instances in shorter term expression is required, or for indications where there is a narrow efficacy/toxicity window.

The invention may relate to the modification of monocytes, iPSC or iPSC-derived cells (such as iPSC-derived monocytes or iPSC-derived epithelial cells)

The invention also relates to populations of immune, immunomodulatory, iPSC and/or immune-derived cells as described herein. Any disclosure relating to the modulation and/or use of immune, immunomodulatory, iPSC and/or immune-derived cells as described herein applies equally and without reservation to populations of said cells.

The lentiviral (e.g. SIV) vectors of the present invention enable long-term transgene expression, resulting in long-term expression of a therapeutic protein by modified immune cells, immunomodulatory cells, iPSCs and/or iPSC-derived (or populations thereof) as described herein. The modified immune cells, immunomodulatory cells, iPSCs and/or iPSC-derived (or populations thereof) used in gene therapy methods of the invention typically express the transgene for the life of the cell. As described herein, the phrases “long-term expression”, “sustained expression”, “long-lasting expression” and “persistent expression” are used interchangeably. Long-term expression according to the present invention means expression of a therapeutic gene and/or protein, preferably at therapeutic levels, for at least 45 days, at least 60 days, at least 90 days, at least 120 days, at least 180 days, at least 250 days, at least 360 days, or more. Preferably long-term expression means expression for at least 45 days, at least 90 days, at least 120 days, at least 180 days, at least 250 days or at least 360 days. In particular, the modified immune cells, immunomodulatory cells, iPSCs and/or iPSC-derived (or populations thereof) of the invention can enable long-term expression of a therapeutic protein in vivo, such as in the lung tissue, epithelial lining fluid and/or serum/plasma.

The immune, immunomodulatory, iPSC and/or immune-derived cells to be modified according to the invention may be obtained or derived from any suitable source. By way of non-limiting example, the immune, immunomodulatory, iPSC and/or immune-derived cells may be derived from peripheral blood, cord blood, bone marrow, fibroblasts or adipose tissue. Typically readily accessible sources, such as peripheral blood mononuclear cells (PBMCs) are preferred. The invention particularly relates to the modification of (differentiated) macrophages, such as macrophages derived from PBMCs or bone marrow. The source of the immune cells, immunomodulatory cells, iPSC, and/or iPSC-derived cells may be obtained from a sample that has been cryopreserved, and/or the immune cells, immunomodulatory cells, iPSC, and/or iPSC-derived cells may be cryopreserved prior to use in an ex vivo method of the invention.

The immune, immunomodulatory, iPSC and/or immune-derived cells to be modified according to the invention may be naïve or may have been previously mobilised. In particular, the immune, immunomodulatory, iPSC and/or immune-derived cells may be obtained from an individual whose cells have not been mobilised by an extrinsically applied factor, such as extrinsically applied G-CSF or GM-CSF. Such immune, immunomodulatory, iPSC and/or immune-derived cells are referred to interchangeably as naïve or non-mobilised. Alternatively, an individual's cells may be mobilised by an extrinsically applied factor, such as extrinsically applied G-CSF or GM-CSF. Such immune, immunomodulatory, iPSC and/or immune-derived cells are referred to as mobilised. Mobilisation may be carried out to increase the number of immune, immunomodulatory, iPSC and/or immune-derived cells obtained for modification according to the present invention.

Methods of Modifying Immune Cells, Immunomodulatory Cells, iPSCs and iPSC-Derived Cells

The inventors have surprisingly shown that, despite the FHN pseudotyping being originally developed to target lentiviral vectors to cells of the airway and lungs, lentiviral (e.g. SIV) vectors pseudotyped with FHN as described herein are also capable of transducing immune cells, immunomodulatory cells, iPSCs and iPSC-derived cells in an efficient manner to achieve long-term high-level transgene expression.

Accordingly, the present invention provides an ex vivo method for obtaining immune cells, immunomodulatory cells, iPSCs and/or iPSC-derived cells modified to express a transgene of interest. Typically said method comprises transducing the immune cells, immunomodulatory cells, iPSCs and/or iPSC-derived cells with a lentiviral vector comprising the transgene.

The transgene may encode an intracellular or membrane protein which will be expressed in or on the surface of the modified immune cell, immunomodulatory cell, iPSC and/or iPSC-derived cell. Alternatively, the transgene may encode a protein which will be secreted by the modified immune cell, immunomodulatory cell, iPSC and/or iPSC-derived cell. When modified immune cells, immunomodulatory cells, iPSCs and/or iPSC-derived cells of the invention are administered to a patient, the secreted protein may either be retained in the lungs (e.g. in the lung epithelial fluid), or may be secreted or pass into the circulatory system, from where it can be distributed systemically or to specific target cells, tissues or organs. Any appropriate transgene may be delivered according to the invention, particularly a transgene with utility in gene therapy, and particularly preferably a transgene for use in gene therapy for a disease or disorder of the lungs or airways. Non-limiting examples of transgenes that may be delivered using lentiviral vectors of the invention are described herein.

Any appropriate lentiviral (e.g. SIV) vector may be used to transduce immune cells, immunomodulatory cells, iPSCs and/or iPSC-derived cells according to the invention. The lentiviral (e.g. SIV) vector used to transduce immune cells, immunomodulatory cells, iPSCs and/or iPSC-derived cells according to the invention is typically a lentiviral vector as described herein. Typically lentiviral (e.g. SIV) vectors pseudotyped with (i) haemagglutinin-neuraminidase (HN) and fusion (F) proteins from a respiratory paramyxovirus; or (ii) G glycoprotein from Vesicular Stomatitis Virus (G-VSV) are used in the ex vivo methods of the invention. In particular, when the transgene encodes an intracellular or membrane protein, the lentiviral (e.g. SIV) vector is pseudotyped with (i) HN and F proteins from a respiratory paramyxovirus; or (ii) G glycoprotein from Vesicular Stomatitis Virus (G-VSV), preferably with HN and F proteins from a respiratory paramyxovirus. When the transgene encodes a secreted protein, the lentiviral (e.g. SIV) vector is typically pseudotyped with (i) HN and F proteins from a respiratory paramyxovirus; or (ii) G glycoprotein from Vesicular Stomatitis Virus (G-VSV), preferably with HN and F proteins from a respiratory paramyxovirus.

Accordingly, the invention provides an ex vivo method for obtaining immune cells, immunomodulatory cells, iPSCs or iPSC-derived cells modified to express a transgene of interest, said method comprising transducing the cells with a lentiviral (e.g. SIV) vector comprising the transgene, wherein the transgene is a secreted therapeutic protein.

The invention also provides an ex vivo method for obtaining immune cells, immunomodulatory cells, iPSC, or iPSC-derived cells modified to express a transgene of interest, said method comprising transducing the cells with a lentiviral (e.g. SIV) vector comprising the transgene, wherein the lentiviral (e.g. SIV) vector is pseudotyped with HN and F proteins from a respiratory paramyxovirus or G-VSV, preferably HN and F proteins from a respiratory paramyxovirus. Said transgene may encode an intracellular protein, a membrane protein or a secreted protein.

The ex vivo methods of the invention may be used to modify any immune cell, immunomodulatory cell, iPSC and/or iPSC-derived cell. Typically the immune cell, immunomodulatory cell, iPSC and/or iPSC-derived cell to be modified is an immune cell, immunomodulatory cell, iPSC and/or iPSC-derived cell as described herein. The invention provides ex vivo methods for obtaining modified mononuclear cells, such as PBMCs or mononuclear cells from cord blood. In particular, the ex vivo methods of the invention can be used to obtain modified macrophages and/or monocytes. The invention also provides ex vivo methods for obtaining modified MSCs cells. The ex vivo methods of the invention can also be used to obtain modified iPSCs or iPSC-derived cells (e.g. epithelial cells derived from iPSCs).

The methods of the invention can be used to modify differentiated cells or progenitor (non-differentiated) cells as described herein. By way of non-limiting example, a preferred ex vivo method of the invention may be used to modify differentiated macrophages. The macrophages to be modified may optionally be differentiated from monocytes, iPSCs or MSCs prior to transduction. In other words, progenitor cells may be obtained (e.g. monocytes or MSCs from a patient or healthy individual, iPSC may be generated from cells from a patient or healthy individual) and differentiated to macrophages, and the differentiated macrophages then transduced with a lentiviral (e.g. SIV) vector according to the invention. Alternatively, differentiated macrophages may be obtained directly (e.g. from a patient or healthy individual), and then then transduced with a lentiviral (e.g. SIV) vector according to the invention.

By way of a further non-limiting example, an ex vivo method of the invention may be used to modify progenitor cells, such as monocytes, iPSCs or MSCs, which are then differentiated (e.g. to macrophages) after the progenitor cells have been transduced with the lentiviral (e.g. SIV) vector. In other words, progenitor cells may be obtained (e.g. monocytes or MSCs from a patient or healthy individual, iPSC may be generated from cells from a patient or healthy individual) and then transduced with a lentiviral (e.g. SIV) vector according to the invention. The modified cells may be differentiated (e.g. to macrophages) prior to administration to a patient. Alternatively, the modified progenitor cells, such as monocytes, iPSCs or MSCs may be administered to a patient without differentiation.

By way of a further non-limiting example, an ex vivo method of the invention may be used to modify iPSC-derived cells (e.g. iPSC-derived epithelial cells), and the iPSC-derived cells are then transduced with the lentiviral (e.g. SIV) vector. In other words, iPSCs may be obtained (e.g. iPSC may be generated from cells from a patient or healthy individual) and differentiated to iPSC-derived cells (e.g. iPSC-derived epithelial cells), and the differentiated iPSC-derived cells (e.g. iPSC-derived epithelial cells) then transduced with a lentiviral (e.g. SIV) vector according to the invention to produce modified iPSC-derived cells (e.g. iPSC-derived epithelial cells).

The ex vivo methods of the invention may comprise one or more additional step, such one or more expansion step, isolation step, concentration step, enrichment step, purification step, and/or formulation step, or a combination thereof. In particular, an ex vivo method of the invention may comprise one or more expansion step and one or more isolation/concentration step as described herein.

An expansion step may be carried out to provide sufficient number of cells. Any appropriate means or protocol for expanding the immune cells, immunomodulatory cells, iPSC, and/or iPSC-derived cells may be used. Such expansion protocols are known in the art and are within the routine skill of one of skill in the art. By way of non-limiting example, expansion may involve the culturing (in vitro or ex vivo) of immune cells, immunomodulatory cells, iPSC, and/or iPSC-derived cells with one or more cytokine or other growth factor. Non-limiting examples of such cytokines or growth factors include M-CSF, IL-3 and epidermal growth factor (EGF). The ex vivo methods of the invention may comprise one or more step of expanding the immune cells, immunomodulatory cells, iPSC, and/or iPSC-derived cells prior to transduction with a lentiviral (e.g. SIV) vector. An expansion step prior to transduction of immune cells, immunomodulatory cells, iPSC, and/or iPSC-derived cells with a lentiviral (e.g. SIV) vector may be carried out to provide a sufficient number of cells for transduction. The ex vivo methods of the invention may comprise one or more step of expanding the immune cells, immunomodulatory cells, iPSC, and/or iPSC-derived cells after transduction with a lentiviral (e.g. SIV) vector. In other words, the ex vivo methods may comprise a step of expanding the modified immune cells, immunomodulatory cells, iPSC, and/or iPSC-derived cells. An expansion step after transduction of immune cells, immunomodulatory cells, iPSC, and/or iPSC-derived cells with a lentiviral (e.g. SIV) vector may be carried out to provide a sufficient number of modified immune cells, immunomodulatory cells, iPSC, and/or iPSC-derived cells for administering to a patient or for carrying out further applications with the modified immune cells, immunomodulatory cells, iPSC, and/or iPSC-derived cells. The ex vivo methods of the invention may comprise both a step of expanding the immune cells, immunomodulatory cells, iPSC, and/or iPSC-derived cells prior to transduction with a lentiviral (e.g. SIV) vector and a step of expanding the modified immune cells, immunomodulatory cells, iPSC, and/or iPSC-derived cells following transduction with the lentiviral (e.g. SIV vector). The one or more expansion step may involve expansion for about 1, 2, 3, 4, 5, 6, 7, 8, 9, 10, 11, 12, 13, 14, 15, 16, 17, 18, 19, 20, 21, 22, 23, 24, 25 days or any range derivable therein either prior to and/or after the transduction step. By way of non-limiting example, the when the immune cells, immunomodulatory cells, iPSC, and/or iPSC-derived cells are expanded prior to transduction, the immune cells, immunomodulatory cells, iPSC, and/or iPSC-derived cells (e.g. macrophages) may be expanded for about days 3, 4, 5, 6 or 7 prior to transduction with a lentiviral (e.g. SIV) vector. The concentration of immune cells, immunomodulatory cells, iPSC, and/or iPSC-derived cells may be kept at an optimum throughout expansion. For instance, progenitor cells can expand up to ˜1500 fold compared to a mononuclear cell population which expands only ˜10-20 fold. Progenitor cells have a large proliferative capacity, as such, where culture is performed in a closed system such a system must provide enough volume for total cell expansion. However, progenitor cells may also have a relatively high inoculation density. Optimal inoculation density and proliferation conditions can be achieved by growing the cells in a bioreactor. An expansion step may typically be included if the unmodified immune cells, immunomodulatory cells, iPSC, and/or iPSC-derived cells are derived from a patient whose cells have not been mobilised (as described herein).

As a non-limiting example, human iPSCs can be cultured under in vitro differentiation protocols s to continuously produce large quantities of terminally differentiated macrophages/monocytes using embryoid-body based differentiation protocols (examples of which are known in the art) and the addition of cytokines IL-3, M-CSF, or GM-CSF.

As a further non-limiting example, to enable expansion of macrophages in culture, an expansion step may induce self-renewal in the macrophages, monocytes or other myeloid precursors. Such an expansion step could involve altering the expression of transcription factors, such as Hoxb8, in myeloid progenitors or monocytes prior to M-CSF or GM-CSF driven macrophage differentiation. Any appropriate means for inducing self-renewal of said cells may be used. Again, suitable techniques and means are known in the art.

The ex vivo methods of the invention may comprise one or more step to isolate and/or concentrate the immune cells, immunomodulatory cells, iPSC, and/or iPSC-derived cells. Said one or more isolation and/or concentration step may be carried out prior to and/or after the immune cells, immunomodulatory cells, iPSC, and/or iPSC-derived cells are transduced with the lentiviral (e.g. SIV) vector. Said one or more isolation and/or concentration step may be carried out prior to or after a step of expanding the immune cells, immunomodulatory cells, iPSC, and/or iPSC-derived cells. By way of non-limiting example, a first isolation and/or concentration step may be carried out after expansion of the unmodified immune cells, immunomodulatory cells, iPSC, and/or iPSC-derived cells, and a second isolation and/or concentration step may be carried out after the immune cells, immunomodulatory cells, iPSC, and/or iPSC-derived cells have been transduced with the lentiviral (e.g. SIV) vector. Any method useful for isolating/concentrating the immune cells, immunomodulatory cells, iPSC, and/or iPSC-derived cells may be used. For example, such isolation/concentration may be based on surface marker expression, which may comprise positive selection of expression and/or negative selection of lineage-specific marker expression. The selection methods may include Magnetic-activated cell sorting (MACS®) or Fluorescence Activated Cell Sorting (FACS™, i.e., flow cytometry). By way of non-limiting example to isolate/concentrate macrophages, markers specific for macrophages may be used. Examples of such markers include, but are not limited to, CD14, CD16, CD64, CD71, CCR5, CD11b, CD11c, CD206, CD69, CD80 and/or CD86.

A population of immune cells, immunomodulatory cells, iPSC, and/or iPSC-derived cells may comprise at least, about, or at most, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶ or 2×10⁶ immune cells, immunomodulatory cells, iPSC, and/or iPSC-derived cells or any range derivable therein. In certain aspects, starting immune cells, immunomodulatory cells, iPSC, and/or iPSC-derived cells prior to expansion or transduction with a lentiviral (e.g. SIV) vector may comprise at least or about 10³, 10⁴, 10⁵, 10⁶, 10⁷, 10⁸, 10⁹, 10¹⁰, 10¹¹, 10¹², 10¹³ cells or any range derivable therein. For suspension culture, a population of immune cells, immunomodulatory cells, iPSC, and/or iPSC-derived cells prior to transduction may have a seeding density of at least or about 10, 10¹, 10², 10³, 10⁴, 10⁵, 10⁶, 10⁷, 10⁸ cells/ml, or any range derivable therein. For adherent culture, a population of immune cells, immunomodulatory cells, iPSC, and/or iPSC-derived cells prior to transduction may have a seeding density of at least or about 10, 10¹, 10², 10³, 10⁴, 10⁵, 10⁶, 10⁷, 10⁸ cells/cm², or any range derivable therein.

It is also possible to obtain a cell sample from a subject (e.g. a patient or healthy individual) and then to enrich it for a desired cell type. For example, PBMCs can be isolated from blood and MSCs can be isolated from bone marrow. Cells can also be isolated from other cells using a variety of techniques, such as isolation and/or activation with an antibody binding to an epitope on the cell surface of the desired cell type. Another method that can be used includes negative selection using antibodies to cell surface markers to selectively enrich for a specific cell type without activating the cell by receptor engagement.

Any appropriate cell culture medium and conditions may be used for the ex vivo methods of the invention. Standard media and conditions are known in the art. All the steps of an ex vivo method of the invention may be carried out using the same culture medium and/or culture conditions. Alternatively, one or more step of an ex vivo method of the invention may be carried out using different culture medium and/or culture conditions. By way of non-limiting example, if an expansion step is used in an ex vivo method of the invention, a specific expansion medium may be used which comprises one or more cytokine or growth factors which will stimulate the expansion of the desired immune cells, immunomodulatory cells, iPSC, and/or iPSC-derived cells. By way of a further non-limiting example, if an ex vivo method of the invention includes an enrichment step, said enrichment step may comprise the use of a specific culture medium which selects for the desired immune cells, immunomodulatory cells, iPSC, and/or iPSC-derived cells.

Any medium, culture or matrix for any of the steps or sub-steps or throughout the whole process may be xeno-free or defined (as described herein). A medium may be chemically defined. One or more medium or culture for use according to the invention may be free or essentially free of any matrix components, or may include a defined or xeno-free extracellular matrix. For human cells, “xeno-free” means that a medium or culture is free or essentially free of any non-human animal components. Preferably the ex vivo methods of the invention are in accordance with GMP and use xeno-free and/or defined culture and media.

Immune cells, immunomodulatory cells, iPSC, and/or iPSC-derived cells may be transduced with a lentiviral (e.g. SIV) vector at any appropriate time point during culture. Typically, a lentiviral (e.g. SIV) vector may be added at a set time point following the start of the ex vivo method or culture of the immune cells, immunomodulatory cells, iPSC, and/or iPSC-derived cells. For example, a lentiviral (e.g. SIV) vector may be following isolation or expansion. For example, a lentiviral (e.g. SIV) vector may be added to the immune cells, immunomodulatory cells, iPSC, and/or iPSC-derived cells on day 3, day 4, day 5, day 6, day 7, day 8, day 9 or day 10 of culture, preferably on day 7, day 5, day 6 or day 8. In other words, transduction may occur on day 3, day 4, day 5, day 6, day 7, day, 8, day 9 or day 10 of the ex vivo method, preferably on day 7, day 5, day 6 or day 8. As described herein, transduction can be carried out on differentiated cells or progenitor cells. Transduction can therefore take place following differentiation of the immune cells, immunomodulatory cells, iPSC, and/or iPSC-derived cells. By way of example, when an ex vivo method of the invention begins (on day 0) with a step of differentiating the immune cells, immunomodulatory cells, iPSC, and/or iPSC-derived cells, transduction may occur (i.e. the lentiviral (e.g. SIV) vector added) once the differentiation step has completed, typically on day 3, day 4, day 5, day 6, day 7, day 8, day 9 or day 10 of culture, preferably on day 7, day 5, day 6 or day 8. Alternatively, transduction can be carried out (the lentiviral (e.g. SIV) vector added) during the process of differentiation. For example, when an ex vivo method of the invention begins (on day 0) with a step of differentiating the immune cells, immunomodulatory cells, iPSC, and/or iPSC-derived cells, a lentiviral (e.g. SIV) vector of the invention may be added to on day 0, day 1, day 2, day 3, day 4, day 5, day 6, or day 7. The lentiviral (e.g. SIV) vector may be added during the differentiation step of at least one day, at least two days, at least three days, at least four days, at least five days, at least six days or at least seven days, typically starting on day 0 if the method.

Any appropriate amount of lentiviral (e.g. SIV) vector may be used to transduce immune cells, immunomodulatory cells, iPSC, and/or iPSC-derived cells according to the present invention. By way of non-limiting example, the immune cells, immunomodulatory cells, iPSC, and/or iPSC-derived cells are transduced with the lentiviral (e.g. SIV) vector of the invention at a MOI of at least about 2, at least about 5, at least about 10, at least about 15, at least about 20, at least about 25, at least about 30, at least about 40, at least about 50, at least about 75, at least about 100 or more. Preferably the immune cells, immunomodulatory cells, iPSC, and/or iPSC-derived cells are transduced with the lentiviral (e.g. SIV) vector of the invention at a MOI of at least about 20, such as at least about 50.

Transduction efficiency of immune cells, immunomodulatory cells, iPSC, and/or iPSC-derived cells with a lentiviral (e.g. SIV) vector of the invention may be at least 5%, at least 10%, at least 15%, at least 20%, at least 25%, at least 30%, at least 35%, at least 40% or more. Preferably transduction efficiency of immune cells, immunomodulatory cells, iPSC, and/or iPSC-derived cells with a lentiviral (e.g. SIV) vector of the invention is at least 10%.

Vector copy number (VCN) of immune cells, immunomodulatory cells, iPSC, and/or iPSC-derived cells following transduction with a lentiviral (e.g. SIV) vector of the invention may be at least 1, at least 2, at least 3%, at least 4, or more.

Modified immune cells, immunomodulatory cells, iPSC, and/or iPSC-derived cells may be harvested at any appropriate time point following transduction with the lentiviral (e.g. SIV) vector. For example, modified immune cells, immunomodulatory cells, iPSC, and/or iPSC-derived cells may be harvested one day, two days, three days, four days, five days, six days, seven days, eight days, nine days, ten days, 12 days, 14 days, 16 days, 18 days, 20 days or later following transduction with the lentiviral (e.g. SIV) vector, preferably on or after seven days following transduction with the lentiviral (e.g. SIV) vector, such as 14 or 16 days following transduction with the lentiviral (e.g. SIV vector). Harvesting of modified immune cells, immunomodulatory cells, iPSC, and/or iPSC-derived cells may comprise or consist of the isolation and/or concentration of the modified immune cells, immunomodulatory cells, iPSC, and/or iPSC-derived cells. Standard techniques for the isolation and/or concentration of a desired cell type are known in the art and any appropriate technique may be used. For example, modified immune cells, immunomodulatory cells, iPSC, and/or iPSC-derived cells may be isolated and/or concentrated on the basis of their expression of the transgene, and/or based on their specific marker profile. Markers for specific immune cells, immunomodulatory cells, iPSC, and/or iPSC-derived cells are known in the art and it is within the routine practice of one of ordinary skill in the art to select suitable markers for the desired cell type. By way of non-limiting example, bone marrow derived macrophages or monocytes may be identified using one or more of CD14, CD16, CD64, CD71, CCR5, CD11b, CD11c, CD206, CD69, CD80 and/or CD86 as markers, or any combination thereof. Other means of identification and/or selection may be used to facilitate the isolation, concentration and/or harvesting of modified immune cells, immunomodulatory cells, iPSC, and/or iPSC-derived cells. For example, modified immune cells, immunomodulatory cells, iPSC, and/or iPSC-derived cells may be identified and/or selected based on their cellular morphology, growth properties and/or cell potency state.

The lentiviral (e.g. SIV) vectors for use in the ex vivo methods of the invention may be produced by any appropriate means. The production of lentiviral (e.g. SIV) vectors typically employs one or more plasmids which provide the elements needed for the production of the vector: the genome for the retroviral/lentiviral vector, the Gag-Pol, Rev, F and HN. Multiple elements can be provided on a single plasmid. Preferably each element is provided on a separate plasmid, such that there are five plasmids, one for each of the vector genome (pDNA1), the Gag-Pol (pDNA2a), Rev (pDNA2b), F (pDNA3a) and HN (pDNA3b), respectively.

Alternatively, a single plasmid may provide the Gag-Pol and Rev elements, and may be referred to as a packaging plasmid (pDNA2). The remaining elements (genome, F and HN) may be provided by separate plasmids (pDNA1, pDNA3a, pDNA3b respectively), such that four plasmids are used for the production of a retroviral/lentiviral (e.g. SIV) vector according to the invention.

Exemplary production methods using four or five plasmids systems are described in WO 2015/177501, which is herein incorporated by reference in its entirety, particularly the pDNA1, pDNA2a, pDNA2b, pDNA3a and pDNA3b described therein. UK Application No. 2102832.9 describes an optimised production method and optimised pDNA2a and pDNA1 plasmids. Again, UK Application No. 2102832.9 is herein incorporated by reference in its entirety, particularly the optimised pDNA2a and pDNA1 plasmids and optimised production method.

Typically the lentiviral (e.g. SIV) vector of the invention gains entry to the immune cells, immunomodulatory cells, iPSCs and/or iPSC-derived cells by virtue of its pseudotyping as described herein (e.g. using the FHN or G-VSV proteins). Typically the lentiviral (e.g. SIV) vector of the invention does not rely on phagocytosis by the immune cells, immunomodulatory cells, iPSCs and/or iPSC-derived cells to gain entry to these cells.

### Lentiviral Vectors

The invention relates to the modification of immune cells, immunomodulatory cells, iPSCs or iPSC-derived cells using a lentiviral (e.g. SIV) construct. The term “lentivirus” refers to a family of retroviruses. Examples of lentiviruses suitable for use in the present invention include Simian immunodeficiency virus (SIV), Human immunodeficiency virus (HIV), Feline immunodeficiency virus (FIV), Equine infectious anaemia virus (EIAV), and Visna/maedi virus. A particularly preferred lentiviral vector is an SIV vector (including all strains and subtypes), such as a SIV-AGM (originally isolated from African green monkeys, Cercopithecus aethiops).

The lentiviral (e.g. SIV) vectors of the present invention are typically pseudotyped with hemagglutinin-neuraminidase (HN) and fusion (F) proteins from a respiratory paramyxovirus, or with G glycoprotein from Vesicular Stomatitis Virus (G-VSV or VSV-G). VSV-G and FHN pseudotypes have surprisingly been shown by the inventors to transduce immune cells, particularly monocytes and macrophages and induce production of a therapeutic proteins. In some embodiments, preferably the lentiviral (e.g. SIV) vectors of the present invention are pseudotyped with HN and F from a respiratory paramyxovirus. Particularly preferably the respiratory paramyxovirus is a Sendai virus (murine parainfluenza virus type 1). In other embodiments, preferably the lentiviral (e.g. SIV) vectors of the present invention are pseudotyped with VSV-G.

A lentiviral (e.g. SIV) vector for use according to the invention may be integrase-competent (IC). Alternatively, the lentiviral (e.g. SIV) vector may be integrase-deficient (ID).

Lentiviral vectors, such as those for use in the methods of the invention, can integrate into the genome of transduced cells and lead to long-lasting expression, making them suitable for transduction of immune and immunomodulatory cells.

Accordingly, lentiviral (e.g. SIV) vectors described herein may transduce one or more cells types as described herein to achieve long term transgene expression. Typically the lentiviral (e.g. SIV) vectors described herein are used to transduce isolated and expanded cells vivo prior administration to a patient.

The lentiviral (e.g. SIV) vectors used according to the present invention enable high levels of transgene expression, resulting in high levels (therapeutic levels) of expression of a therapeutic protein. The lentiviral (e.g. SIV) vectors of the present invention typically provide high expression levels of a transgene when used to transduce immune cells, immunomodulatory cells, iPSCs and/or iPSC-derived ex vivo. Typically this high level of transgene expression is maintained when the modified immune cells, immunomodulatory cells, iPSCs and/or iPSC-derived are administered to a patient. The terms high expression and therapeutic expression are used interchangeably herein. Expression may be measured by any appropriate method (qualitative or quantitative, preferably quantitative), and concentrations given in any appropriate unit of measurement, for example ng/ml or nM.

Expression of a transgene of interest may be given in absolute terms or relative to the expression of the corresponding endogenous (defective) gene. Relative expression may be in terms of the expression of the corresponding endogenous gene in unmodified cells of the same cell type ex vivo, or in terms of the expression of the corresponding endogenous gene in vivo in a patient.

Expression may be measured in terms of mRNA or protein expression. The expression of the transgene of the invention, such as a functional CFTR gene, may be quantified relative to the endogenous gene, such as the endogenous (dysfunctional) CFTR genes in terms of mRNA copies per cell or any other appropriate unit.

Expression levels of a transgene and/or the encoded therapeutic protein of the invention may be measured ex vivo (e.g. in the conditioned media used to culture the cells or within the cells themselves) or in vivo (e.g. in the lung tissue, epithelial lining fluid and/or serum/plasma) as appropriate. A high and/or therapeutic expression level may therefore refer to the concentration in the lung, epithelial lining fluid and/or serum/plasma.

The transgene included in a lentiviral (e.g. SIV) vector of the invention may be modified to facilitate expression. For example, the transgene sequence may be in CpG-depleted (or CpG-fee) and/or codon-optimised form to facilitate gene expression. Standard techniques for modifying the transgene sequence in this way are known in the art.

The lentiviral (e.g. SIV) vectors of the invention exhibit enhanced transgene expression. Accordingly, the lentiviral (e.g. SIV) vectors of the invention have the potential to produce long-lasting, repeatable, high-level expression in immune cells, immunomodulatory cells, iPSCs and/or iPSC-derived cells without inducing an undue immune response.

The lentiviral (e.g. SIV) vectors of the present invention enable long-term transgene expression, resulting in long-term expression of a therapeutic protein by immune cells, immunomodulatory cells, iPSCs and/or iPSC-derived as described herein. Long-term expression according to the present invention means expression of a therapeutic gene and/or protein, preferably at therapeutic levels, for at least 45 days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more.

The lentiviral (e.g. SIV) vector may comprise a promoter operably linked to a transgene, enabling expression of the transgene. By way of non-limiting example, a preferred promoter is a hybrid human CMV enhancer/EF1a (hCEF) promoter. This hCEF promoter may lack the intron corresponding to nucleotides 570-709 and the exon corresponding to nucleotides 728-733 of the hCEF promoter. An example of an hCEF promoter sequence of the invention is provided by SEQ ID NO: 1. The promoter may be a CMV promoter. An example of a CMV promoter sequence is provided by SEQ ID NO: 2. The promoter may be a human elongation factor 1a (EF1a) promoter. An example of a EF1a promoter is provided by SEQ ID NO: 3. Other promoters for transgene expression are known in the art and their suitability for the retroviral/lentiviral (e.g. SIV) vectors of the invention determined using routine techniques known in the art. Non-limiting examples of other promoters include UbC and UCOE. As described herein, the promoter may be modified to further regulate expression of the transgene of the invention.

The promoter included in the lentiviral (e.g. SIV) vector of the invention may be specifically selected and/or modified to further refine regulation of expression of the therapeutic gene. Again, suitable promoters and standard techniques for their modification are known in the art. As a non-limiting example, a number of suitable (CpG-free) promoters suitable for use in the present invention are described in Pringle et al. (J. Mol. Med. Berl. 2012, 90 (12): 1487-96), which is herein incorporated by reference in its entirety. Preferably, the lentiviral vectors (particularly SIV F/HN vectors) of the invention comprise a hCEF promoter having low or no CpG dinucleotide content. The hCEF promoter may have all CG dinucleotides replaced with any one of AG, TG or GT. Thus, the hCEF promoter may be CpG-free. A preferred example of a CpG-free hCEF promoter sequence of the invention is provided by SEQ ID NO: 1. The absence of CpG dinucleotides further improves the performance of lentiviral (e.g. SIV) vectors of the invention and in particular in situations where it is not desired to induce an immune response against an expressed antigen or an inflammatory response against the delivered expression construct. The elimination of CpG dinucleotides reduces the occurrence of flu-like symptoms and inflammation which may result from administration of constructs, particularly when administered to the airways.

The lentiviral (e.g. SIV) vector of the invention may be modified to allow shut down of gene expression. Standard techniques for modifying the vector in this way are known in the art. As a non-limiting example, Tet-responsive promoters are widely used.

Expression of the transgene in a lentiviral (e.g. SIV) vector of the invention may be controlled using a regulated promoter, particularly a steroid-regulated promoter. Steroid-regulated promoter systems are known in the art, with suitable systems being commercially available (e.g. the GeneSwitch™ system by Thermo Fisher). Use of such steroid-regulated promoters with lentiviral (e.g. SIV) vector of the invention is within the routine practice of one of ordinary skill in the art, and is demonstrated herein in the context of driving transgene expression in macrophages.

Steroid-regulated promoter systems typically comprise a regulated promoter (which can replace hCEF or any of the other promoters in the plasmids described herein) and a transactivator (which may be encoded by a regulatory plasmid or by any of the plasmids described herein). Preferably the vector genome plasmid (pDNA1) comprises a transgene operably linked to an regulated promoter. The pDNA1 may further encodes the corresponding trans-activator Thus, the transgene operably linked to the regulated promoter and the trans-activator can be encoded by a single lentiviral (e.g. SIV) vector. In the single vector system, the (i) transgene operably linked to the regulated promoter and (ii) the gene encoding the trans-activator are present in the same vector backbone, typically in opposite orientations. Alternatively, the transgene operably linked to the regulated promoter may be encoded by a first lentiviral (e.g. SIV) vector and the trans-activator may be encoded by a second lentiviral (e.g. SIV) vector. Both these configurations are exemplified herein. Preferably, a two vector system is used, i.e. the trans-activator is encoded on a second/separate lentiviral (e.g. SIV) vector to the transgene operably linked to the regulated promoter.

In a patient, transgene expression by transplanted immune cells, immunomodulatory cells, iPSCs and/or iPSC-derived cells may be initiated by administration of the appropriate activating agent, such as the appropriate steroid when using a steroid-regulated promoter (mifepristone in the case of a mifepristone-regulated promoter, such as GeneSwitch™, or a one vector variation thereof).

One non-limiting example of a steroid-regulated promoter which maybe used with the present invention is a mifepristone-regulated promoter, such as the commercially available GeneSwitch™. This exemplary mifepristone-regulated promoter has the following structure: (i) a GAL4 upstream activating sequence (UAS), which may comprise six GAL4 binding site; (ii) the adenovirus E1b TATA box; and (iii) an intron (e.g. the synthetic intron IVS8). A non-limiting example of a mifepristone-regulated promoter sequence is found in SEQ ID NO: 26. An exemplary trans-activator for use with a mifepristone-regulated promoter may have the following structure: (i) a GAL4 DNA-binding domain (DBD); (ii) a human progesterone receptor ligand binding domain (IPR-LBD) which binds to mifepristone; and (iii) human NF-κB p65 activation domain (AD). A non-limiting example of a nucleic acid sequence encoding a trans-activator for use with a mifepristone-regulated promoter is found in SEQ ID NO: 27. In the presence of mifepristone, the hPR-LBD domain on the GeneSwitch™ regulatory protein undergoes a conformational change, enabling activation of the GAL4-E1b promoter, resulting in transgene expression. The trans-activator further upregulates its own expression by binding to a Gal4 DNA Binding Domain upstream of the HSV TK promoter, therefore amplifying the induction of expression of the gene of interest.

Preferably, the invention relates to the use of F/HN lentiviral vectors comprising a promoter and a transgene, particularly SIV F/HN vectors.

A lentiviral (e.g. SIV) vector of the invention may comprise a transgene that encodes a polypeptide or protein that is therapeutic for the treatment of such diseases, particularly a disease or disorder of the airways, respiratory tract, or lung.

Accordingly, a lentiviral (e.g. SIV) vector of the invention may comprise a transgene encoding a protein selected from: (i) a secreted therapeutic protein, optionally Granulocyte-Macrophage Colony-Stimulating Factor (GM-CSF), Alpha-1 Antitrypsin (AAT), Factor VIII, Surfactant Protein B (SFTPB), Factor VII, Factor IX, Factor X, Factor XI, von Willebrand Factor, decorin, an anti-inflammatory protein (e.g. IL-10 or TGFβ) or monoclonal antibody, or a monoclonal antibody against an infectious agent; or (ii) a non-secreted protein involved in macrophage biology, optionally CFTR, ABCA3, CSF2RA, CSF2RB or TRIM-72. Other examples of transgenes that may be comprised in a lentiviral (e.g. SIV) vector of the invention include genes related to or associated with other surfactant deficiencies. In some embodiments, the transgene is selected from CSF2, SERPINA1, DCN, TRIM72, ABACA3, FVIII and/or CFTR.

Preferably, the transgene encodes a CFTR. An example of a CFTR transgene is provided by SEQ ID NO: 4. The polypeptide encoded by said CFTR transgene may be exemplified by the polypeptide of SEQ ID NO: 5. Variants thereof (as described therein) are also included, particularly variants with at least 90% (such as at least 90, 92, 94, 95, 96, 97, 98, 99 or 100%) to SEQ ID NO: 4 or 5.

The transgene may encode GM-CSF. An example of a human GM-CSF transgene (CSF2) is provided by SEQ ID NO: 6. An example of a mouse GM-CSF transgene (CSF2) is provided by SEQ ID NO: 8. Preferably a human GM-CSF transgene, such as SEQ ID NO: 6, us used. The polypeptide encoded by said CSF2 transgene, may be exemplified by the polypeptide of SEQ ID NO: 7 (human) or 9 (mouse). Preferably the transgene encodes a human GM-CSF polypeptide, such as SEQ ID NO: 7. Variants thereof (as described therein) are also included, particularly variants with at least 90% (such as at least 90, 92, 94, 95, 96, 97, 98, 99 or 100% to any one of SEQ ID NOs: 6 to 9, preferably SEQ ID NOs: 6 and 7.

The transgene may encode an AAT. An example of a SERPINA1 transgene is provided by SEQ ID NO: 10, or by the complementary sequence of SEQ ID NO: 11. SEQ ID NO: 10 is a codon-optimized CpG depleted SERPINA1 transgene previously designed by the present inventors to enhance translation in human cells. Such optimisation has been shown to enhance gene expression by up to 15-fold. Variants of same sequence (as defined herein) which possess the same technical effect of enhancing translation compared with the unmodified (wild-type) SERPINA1 gene sequence are also encompassed by the present invention. The polypeptide encoded by said SERPINA1. transgene, may be exemplified by the polypeptide of SEQ ID NO: 12. Variants thereof (as described therein) are also included, particularly variants with at least 90% (such as at least 90, 92, 94, 95, 96, 97, 98, 99 or 100% to any one of SEQ ID NOs: 10, 11 or 12.

The transgene may encode a FVIII. Examples of a FVIII transgene are provided by SEQ ID NOs: 13 and 14, or by the respective complementary sequences of SEQ ID NO: 15 and 16. The polypeptide encoded by the FVIII transgene, may be exemplified by the polypeptide of SEQ ID NO: 17 or 18. Variants thereof (as described therein) are also included, particularly variants with at least 90% (such as at least 90, 92, 94, 95, 96, 97, 98, 99 or 100%) to any one of SEQ ID NOs: 13 to 18.

The transgene may encode decorin. An example of a DCN transgene is provided by SEQ ID NO: 19. The polypeptide encoded by said DCN transgene, may be exemplified by the polypeptide of SEQ ID NO: 20. Variants thereof (as described therein) are also included, particularly variants with at least 90% (such as at least 90, 92, 94, 95, 96, 97, 98, 99 or 100% to SEQ ID NO: 19 or 20.

The transgene may encode TRIM72. An example of a TRIM72 transgene is provided by SEQ ID NO: 21. The polypeptide encoded by said TRIM 72 transgene, may be exemplified by the polypeptide of SEQ ID NO: 22. Variants thereof (as described therein) are also included, particularly variants with at least 90% (such as at least 90, 92, 94, 95, 96, 97, 98, 99 or 100% to SEQ ID NO: 21 or 22.

The transgene may encode ABCA3. An example of a ABACA3 transgene is provided by SEQ ID NO: 23. The polypeptide encoded by said ABACA3 transgene, may be exemplified by the polypeptide of SEQ ID NO: 24. Variants thereof (as described therein) are also included, particularly variants with at least 90% (such as at least 90, 92, 94, 95, 96, 97, 98, 99 or 100% to SEQ ID NO: 23 or 24.

The transgene of the invention may be any one or more of DNAH5, DNAH11, DNAI1, and DNAI2, or other known related gene.

Other non-limiting examples of transgenes useful in the treatment of respiratory diseases include SFTPB. The transgene may encode a monoclonal antibody (mAb) against an infectious agent. The transgene may encode anti-TNF alpha. The transgene may encode a therapeutic protein implicated in an inflammatory, allergic, immune or metabolic condition.

A lentiviral (e.g. SIV) vector of the invention may be delivered to the immune cells, immunomodulatory cells, iPSCs and/or iPSC-derived ex vivo to allow for the production of proteins on delivery to a patient that are secreted into the lung epithelial lining fluid and/or the circulatory system. In embodiments particularly relating to the secretion of proteins into the circulatory system, the transgene may encode for Factor VII, Factor VIII, Factor IX, Factor X, Factor XI and/or von Willebrand's factor. Such a vector may be used in the treatment of diseases, particularly cardiovascular diseases and blood disorders, preferably blood clotting deficiencies such as haemophilia. Again, the transgene may encode an anti-inflammatory protein or mAb, or an mAb against an infectious agent or a protein implicated in an inflammatory, allergic, immune or metabolic condition, such as, lysosomal storage disease.

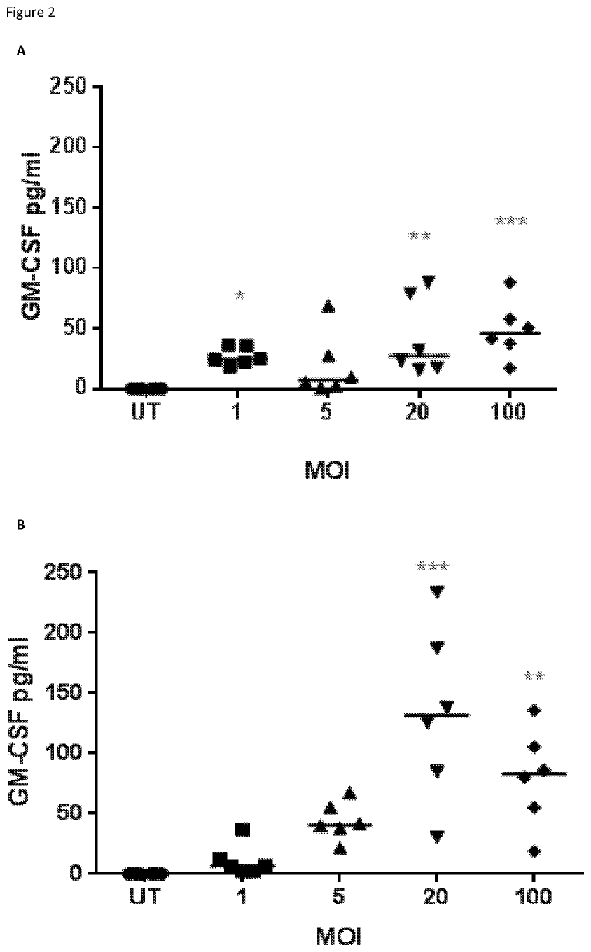

The lentiviral (e.g. SIV) vector of the invention may have no intron positioned between the promoter and the transgene. Similarly, there may be no intron between the promoter and the transgene in the vector genome (pDNA1) plasmid (for example, pGM326 or pGM830 as illustrated in FIGS. 2A and B and the corresponding sequences in UK Application No. 2102832.9, which is herein incorporated by reference in its entirety).

In some preferred embodiments, the lentiviral (e.g. SIV) vector comprises a hCEF promoter and a CFTR transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the CFTR transgene and a promoter.

In some preferred embodiments, the lentiviral (e.g. SIV) vector comprises a hCEF promoter and an GM-CSF transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the GM-CSF transgene and a promoter.

In some preferred embodiments, the lentiviral (e.g. SIV) vector comprises a hCEF promoter and an SERPINA1 (AAT) transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the SERPINA1 (AAT) transgene and a promoter.

In some preferred embodiments, the lentiviral (e.g. SIV) vector comprises a hCEF or CMV promoter and an FVIII transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the FVIII transgene and a promoter.

In some preferred embodiments, the lentiviral (e.g. SIV) vector comprises a hCEF or CMV promoter and an DCN transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the DCN transgene and a promoter.

In some preferred embodiments, the lentiviral (e.g. SIV) vector comprises a hCEF or CMV promoter and an TRIM72 transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the TRIM72 transgene and a promoter.

In some preferred embodiments, the lentiviral (e.g. SIV) vector comprises a hCEF or CMV promoter and an ABACA3 transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the ABACA3 transgene and a promoter.

The lentiviral (e.g. SIV) vector as described herein comprises a transgene. The transgene comprises a nucleic acid sequence encoding a gene product, e.g., a protein, particularly a therapeutic protein.

For example, the nucleic acid sequence encoding a CFTR, GM-CSF, SERPINA1 FVIII, decorin, TRIM72 or ABAC3 comprises (or consists of) a nucleic acid sequence having at least 90% (such as at least 90, 92, 94, 95, 96, 97, 98, 99 or 100%) sequence identity to the CFTR, GM-CSF, SERPINA1, FVIII, decorin, TRIM72 or ABAC3 nucleic acid sequence respectively, examples of which are described herein. In a further embodiment, the nucleic acid sequence encoding CFTR, GM-CSF, SERPINA1, FVIII, decorin, TRIM72 or ABAC3 comprises (or consists of) a nucleic acid sequence having at least 95% (such as at least 95, 96, 97, 98, 99 or 100%) sequence identity to the CFTR, GM-CSF, SERPINA1,FVIII, decorin, TRIM72 or ABAC3 nucleic acid sequence respectively, examples of which are described herein. In one embodiment, the nucleic acid sequence encoding CFTR is provided by SEQ ID NO: 4, the nucleic acid sequence encoding GM-CSF is provided by SEQ ID NO: 6 or 8, preferably SEQ ID NO: 6, the nucleic acid sequence encoding AAT is provided by SEQ ID NO: 10, or by the complementary sequence of SEQ ID NO: 11, the nucleic acid sequence encoding FVIII is provided by SEQ ID NO: 13 or 14, or by the respective complementary sequence of SEQ ID NO: 15 or 16, the nucleic acid sequence encoding decorin is provided by SEQ ID NO: 19, the nucleic acid sequence encoding TRIM72 is provided by SEQ ID NO: 21, and/or the nucleic acid sequence encoding ABCA3 is provided by SEQ ID NO: 24, or variants thereof.

The amino acid sequence of the CFTR, GM-CSF, AAT, FVIII, decorin, TRIM72 and/or ABCA3 polypeptide encoded by the respective CFTR, CSF2, SERPINA1, FVIII, DCN, TRIM72, and/or ABACA3 transgene may comprise (or consist of) an amino acid sequence having at least 95% (such as at least 95, 96, 97, 98, 99 or 100%) sequence identity to the functional CFTR, GM-CSF, AAT, FVIII, decorin, TRIM72 or ABCA3 polypeptide sequence respectively.

The transgene may include a nucleic acid sequence encoding for a signal peptide (such as the endogenous signal peptide of a secreted protein), or may exclude a nucleic acid sequence encoding for a signal peptide. The therapeutic protein may include a signal peptide (such as the endogenous signal peptide of a secreted protein), or may exclude a signal peptide. Where appropriate, endogenous signal peptides have been identified in the sequence information section herein. All disclosure herein relates to both transgenes and therapeutic proteins including and excluding signal peptides unless explicitly stated. By way of non-limiting example, sequence identity of variants, and/or lengths of fragments may be based on the sequence with or without a signal peptide.

The lentiviral (e.g. SIV) vectors of the invention may comprise a central polypurine tract (cPPT) and/or the Woodchuck hepatitis virus posttranscriptional regulatory elements (WPRE). An exemplary WPRE sequence is provided by SEQ ID NO: 25.

### Therapeutic Indications

The invention provides a population of modified immune cells, immunomodulatory cells, iPSCs and/or iPSC-derived cells as defined herein for use in a method of gene therapy.

Said method of gene therapy typically comprises carrying out an ex vivo method for the production of modified immune cells, immunomodulatory cells, iPSCs and/or iPSC-derived cells according to the invention, and administering the modified cells produced by said method (or a population thereof) to a patient.

The method may involve one or more additional step between carrying out an ex vivo method of the invention and the step of administering the modified cells produced by said method (or a population thereof). By way of non-limiting example, the modified cells produced by said method (or a population thereof) may be stored prior to administration to a patient. Storage may be by any appropriate means, such as freezing.

The immune cells, immunomodulatory cells, iPSCs and/or iPSC-derived cells (or populations thereof) to be modified and used in a method of gene therapy may be (a) autologous cells derived from a patient to be treated; or (b) allogenic cells derived from an individual other than the patient. For example, peripheral blood cells that are collected and stored in blood banks could be used as a source of either autologous or allogeneic but histocompatible immune cells, immunomodulatory cells, iPSCs and/or iPSC-derived cells (or populations thereof) to be modified according to the invention.

The modified immune cells, immunomodulatory cells, iPSCs and/or iPSC-derived cells (or populations thereof) used in gene therapy methods of the invention typically express the transgene for the life of the cell. In embodiments where lost-lasting transgene expression within a patient is desired, the type of modified immune cells, immunomodulatory cells, iPSCs and/or iPSC-derived cells (or populations thereof) may be selected such that the modified immune cells, immunomodulatory cells, iPSCs and/or iPSC-derived cells (or populations thereof) persist within a patient for at least 14 days, at least 21 days, at least 28 days, at least 32 days, at least 45 days, at least 60 days, at least 72 days, at least 90 days, at least 120 days, at least 160 days, at least 220 days, at least 360 days or more. In such embodiments, preferably, the modified immune cells, immunomodulatory cells, iPSCs and/or iPSC-derived cells (or populations thereof) may persist within a patient for at least 28 days, more preferably at least 45 days. In embodiments where a limited duration of transgene expression within a patient is desired, the type of modified immune cells, immunomodulatory cells, iPSCs and/or iPSC-derived cells (or populations thereof) may be selected such that the modified immune cells, immunomodulatory cells, iPSCs and/or iPSC-derived cells (or populations thereof) persist within a patient for a maximum of 90 days, a maximum of 72 days, a maximum of 60 days, a maximum of 45 days, a maximum of 32 days, a maximum of 28 days, a maximum of 21 days, a maximum of 14 days, or less. In such embodiments, preferably, the modified immune cells, immunomodulatory cells, iPSCs and/or iPSC-derived cells (or populations thereof) may persist within a patient for a maximum of 45 days, more preferably for a maximum of 28 days.

By way of non-limiting example, the modified immune cells, immunomodulatory cells, iPSCs and/or iPSC-derived cells (or populations thereof) may be modified immune cells, optionally monocytes or macrophages. The modified immune cells may persist within a patient for at least 14 days, at least 21 days, at least 28 days, at least 32 days, at least 45 days, at least 60 days, at least 72 days, at least 90 days, at least 120 days, at least 160 days, at least 220 days, at least 360 days or more. Preferably, the modified immune cells may persist within a patient for at least 28 days, more preferably at least 45 days. Thus, the transgene expressed by the modified immune cells may be expressed within the patient (e.g. within the modified immune cells, in the lung epithelial fluid and/or the blood/serum as described herein) for at least 14 days, at least 21 days, at least 28 days, at least 32 days, at least 45 days, at least 60 days, at least 72 days, at least 90 days, at least 120 days, at least 160 days, at least 360 days or more. Preferably the transgene expressed by the modified immune cells is expressed for at least 28 days, more preferably at least 45 days.

By way of a further non-limiting example, the modified immune cells, immunomodulatory cells, iPSCs and/or iPSC-derived cells (or populations thereof) used in gene therapy methods of the invention may be modified iPSC-derived cells, optionally mesenchymal stem cells (MSCs). The modified iPSC-derived cells (e.g. MSC) s may persist within a patient for a maximum of 90 days, a maximum of 72 days, a maximum of 60 days, a maximum of 45 days, a maximum of 32 days, a maximum of 28 days, a maximum of 21 days, a maximum of 14 days, or less. Preferably, the modified iPSC-derived cells (e.g. MSCs) may persist within a patient for a maximum of 45 days, more preferably for a maximum of 28 days. Thus, the transgene expressed by the modified iPSC-derived cells (e.g. MSCs) may be expressed within the patient (e.g. within the modified iPSC-derived cells (e.g. MSCs), in the lung epithelial fluid and/or the blood/serum as described herein) for a maximum of 90 days, a maximum of 72 days, a maximum of 60 days, a maximum of 45 days, a maximum of 32 days, a maximum of 28 days, a maximum of 21 days, a maximum of 14 days, or less. Preferably the transgene expressed by the modified immunoregulatory cells is expressed for a maximum of 45 days, more preferably for a maximum of 28 days.

Repeated doses of modified immune cells, immunomodulatory cells, iPSCs and/or iPSC-derived cells (or populations thereof) may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated.

The modified immune cells, immunomodulatory cells, iPSCs and/or iPSC-derived cells (or populations thereof) may be used to deliver any transgene useful in gene therapy. Typically, the modified immune cells, immunomodulatory cells, iPSCs and/or iPSC-derived cells (or populations thereof) of the invention are for use in gene therapy for the treatment of a disease or disorder of the airways, respiratory tract, or lung. Typically, the modified immune cells, immunomodulatory cells, iPSCs and/or iPSC-derived cells (or populations thereof) may be used for the treatment of a genetic respiratory disease.

The disease to be treated may be chronic or acute. Examples of lung diseases for treatment according to the invention include Cystic Fibrosis (CF), Alpha 1-antitrypsin Deficiency (A1AD), Pulmonary Alveolar Proteinosis (PAP, hereditary and/or acquired), Chronic Obstructive Pulmonary Disease (COPD), a surfactant deficiency, primary ciliary dyskinesia (PCD), an infection of the lung (acute or chronic), an inflammatory condition of the lung (acute or chronic), an allergic condition of the lung (acute or chronic), asthma, lung cancer, a fibrotic condition of the lung (including idiopathic pulmonary fibrosis. IPF), or any other lung disease or disorder. Expression of the therapeutic protein may be detected in the lung tissue or in bronchoalveolar lavage fluid (BALF). The modified immune cells, immunomodulatory cells, iPSCs and/or iPSC-derived cells (or populations thereof) may be used in gene therapy to treat a systemic condition, wherein the lung is used as a factory to produce a protein which is secreted from the lung into the circulatory system. The therapeutic protein may therefore be detected at therapeutic levels in the blood/serum.

The invention also provides a lentiviral (e.g. SIV) vector as described herein for use in a method of gene therapy, wherein said method comprises the steps of: (a) transducing immune cells, immunomodulatory cells, iPSCs and/or iPSC-derived cells with the lentiviral vector to produce modified immune cells, immunomodulatory cells, iPSCs and/or iPSC-derived cells expressing a transgene of interest; and (b) administering the resulting modified immune cells or immunomodulatory cells to a patient. Typically, said method of gene therapy comprises carrying out the ex vivo method of the invention and administering the modified cells produced by said method to a patient.

The invention also provides a composition comprising a population of modified immune cells, immunomodulatory cells, iPSCs and/or iPSC-derived cells comprising a transgene of interest according to the invention, and optionally a pharmaceutically acceptable excipient, buffer or diluent.

The invention also provides the use of a population of modified immune cells, immunomodulatory cells, iPSCs and/or iPSC-derived cells according to the invention for use in a method of gene therapy.

The invention also provides a gene therapy method comprising administering to a subject in need thereof a therapeutically effective amount of a population of modified immune cells, immunomodulatory cells, iPSCs and/or iPSC-derived cells comprising a transgene of interest, which cells are obtainable by an ex vivo method of the invention as described herein.

The invention also provides the use of a population of immune cells, immunomodulatory cells, iPSCs and/or iPSC-derived cells as described herein in the manufacture of a medicament for gene therapy.

The invention further provides the use of a lentiviral (e.g. SIV) vector as described herein for use in the manufacture of a medicament for gene therapy, wherein the medicament is a population of modified immune cells, immunomodulatory cells, iPSCs and/or iPSC-derived cells as described herein, typically produced by an ex vivo method of the invention.

Any and all disclosure herein in relation to the modified immune cells, immunomodulatory cells, iPSCs and/or iPSC-derived cells (or populations thereof) and/or lentiviral vectors for use in a method of gene therapy applies equally and without reservation to the therapeutic uses and methods described herein.

The modified immune cells, immunomodulatory cells, iPSCs and/or iPSC-derived cells (or populations thereof) of the invention may be administered in any dosage appropriate for achieving the desired therapeutic effect. Appropriate dosages may be determined by a clinician or other medical practitioner using standard techniques and within the normal course of their work.

The lentiviral (e.g. SIV) vectors of the present invention enable high and sustained gene expression through efficient gene transfer to immune cells, immunomodulatory cells, iPSCs and/or iPSC-derived cells. This is surprising, given that lentiviral vectors with an F/HN pseudotype was originally designed to specifically transduce cells within the airway epithelium.

Long term/persistent stable gene expression, preferably at a therapeutically-effective level, may be achieved using repeat doses of modified immune cells, immunomodulatory cells, iPSCs and/or iPSC-derived cells (or populations thereof) of the present invention. Alternatively, a single dose may be used to achieve the desired long-term expression.

Thus, advantageously, the retroviral/lentiviral (e.g. SIV) vectors of the present invention can be used to produce modified immune cells, immunomodulatory cells, iPSCs and/or iPSC-derived cells (or populations thereof) suitable for use in gene therapy. The modified immune cells, immunomodulatory cells, iPSCs and/or iPSC-derived cells (or populations thereof) are highly suitable for treating respiratory tract diseases. The modified immune cells, immunomodulatory cells, iPSCs and/or iPSC-derived cells (or populations thereof) of the invention can also be used in methods of gene therapy to promote secretion of therapeutic proteins. By way of further example, the invention provides secretion of therapeutic proteins into the lumen of the respiratory tract or the circulatory system. Thus, administration of modified immune cells, immunomodulatory cells, iPSCs and/or iPSC-derived cells (or populations thereof) of the invention may enable the use of the lungs (or nose or airways) as a “factory” to produce a therapeutic protein that is then secreted and enters the general circulation at therapeutic levels, where it can travel to cells/tissues of interest to elicit a therapeutic effect. Thus, other diseases which are not respiratory tract diseases, such as cardiovascular diseases and blood disorders, particularly blood clotting deficiencies, can also be treated by the modified immune cells, immunomodulatory cells, iPSCs and/or iPSC-derived cells (or populations thereof) of the present invention.

Modified immune cells, immunomodulatory cells, iPSCs and/or iPSC-derived cells (or populations thereof) of the invention can effectively treat a disease by expressing a transgene for the correction of the disease. For example, inserting a functional copy of the CFTR gene into macrophages ex vivo and delivery of such modified macrophages may ameliorate or prevent lung disease in CF patients, independent of the underlying mutation. Accordingly, modified immune cells, immunomodulatory cells, iPSCs and/or iPSC-derived cells (or populations thereof) of the invention may be used to treat cystic fibrosis (CF), typically by gene therapy with a CFTR transgene as described herein. In particular, modified macrophages (or modified monocytes or other progenitor cells that can be differentiated to macrophages either before or after administration to a patient) expressing a CFTR transgene may be of particular use in the treatment of CF.

As another example, modified immune cells, immunomodulatory cells, iPSCs and/or iPSC-derived cells (or populations thereof) of the invention may be used to treat Alpha-1 Antitrypsin (AAT) deficiency, typically by gene therapy with an AAT transgene, SERPINA1, as described herein. AAT is a secreted anti-protease that is produced mainly in the liver and then trafficked to the lung, with smaller amounts also being produced in the lung itself. The main function of AAT is to bind and neutralise/inhibit neutrophil elastase. Gene therapy with AAT according to the present invention is relevant to AAT deficient patient, as well as in other lung diseases such as CF or chronic obstructive pulmonary disease (COPD), and offers the opportunity to overcome some of the problems encountered by conventional enzyme replacement therapy (in which AAT isolated from human blood and administered intravenously every week). Delivery of modified cells to a target tissue where those cells can express a therapeutic protein (either within the modified cells or for secretion therefrom) has the potential to provide stable, long-lasting expression in the target tissue, ease of administration and unlimited availability.

Treatment of a patient with modified immune cells, immunomodulatory cells, iPSCs and/or iPSC-derived cells (or populations thereof) of the invention may lead to secretion of the recombinant protein into the lumen of the lung as well as into the circulation. One benefit of this is that the therapeutic protein reaches the interstitium. AAT gene therapy may therefore also be beneficial in other disease indications, non-limiting examples of which include type 1 and type 2 diabetes, acute myocardial infarction, ischemic heart disease, rheumatoid arthritis, inflammatory bowel disease, transplant rejection, graft versus host (GvH) disease, multiple sclerosis, liver disease, cirrhosis, vasculitides and infections, such as bacterial and/or viral infections.

AAT has numerous other anti-inflammatory and tissue-protective effects, for example in pre-clinical models of diabetes, graft versus host disease and inflammatory bowel disease. The production of AAT in the lung and/or nose following cell delivery according to the present invention may, therefore, be more widely applicable, including to these indications.

Other examples of diseases that may be treated with gene therapy of a secreted protein according to the present invention include cardiovascular diseases and blood disorders, particularly blood clotting deficiencies such as haemophilia (A, B or C), von Willebrand disease and Factor VII deficiency.

Other examples of diseases or disorders to be treated include Pulmonary Alveolar Proteinosis (PAP), Primary Ciliary Dyskinesia (PCD), acute lung injury, surfactant deficiencies, Chronic Obstructive Pulmonary Disease (COPD) and/or inflammatory, infectious, immune or metabolic conditions, such as lysosomal storage diseases, as described herein.

PAP is a pulmonary alveoli-filling disease, characterized by dense phospholipoproteinaceous deposits in the alveoli, cough, and dyspnea. A primary cause of PAP is (congenital or acquired) impaired processing of pulmonary surfactants by alveolar macrophages, a process dependent on GM-CSF. There are 3 clinically distinct forms of PAP: hereditary (usually congenital), secondary, and acquired, with the acquired form being most prevalent. Secondary pulmonary alveolar proteinosis is associated with functional impairment or reduced numbers of alveolar macrophages, as occurs in some hematologic cancers, immunosuppression, inhalation of inorganic dust or toxic fumes, and certain infections. Congenital pulmonary alveolar proteinosis is a rare, severe, often fatal disorder of newborns associated with pulmonary surfactant metabolism dysfunction caused by mutations in genes involved in surfactant metabolism. Modified immune cells, immunomodulatory cells, iPSCs and/or iPSC-derived cells (or populations thereof) expressing GM-CSF may therefore be useful in the treatment of PAP.

Idiopathic pulmonary fibrosis (IPF) is a common interstitial lung disease of unknown etiology, usually occurring between 50-70 years of age. Clinically, it is characterized by an insidious onset of breathlessness with exertion and a nonproductive cough, leading to progressive dyspnea. Pathological features show scant interstitial inflammation, patchy collagen fibrosis, prominent fibroblast proliferation foci, and microscopic honeycomb change. Modified immune cells, immunomodulatory cells, iPSCs and/or iPSC-derived cells (or populations thereof) expressing tripartite motif-containing protein 72 (TRIM-72) and/or decorin may be useful in the treatment of IPF.

### Formulation and Administration

The invention also provides compositions comprising modified immune cells, immunomodulatory cells, iPSCs and/or iPSC-derived cells (or populations thereof) as described above, and a pharmaceutically-acceptable carrier. Non-limiting examples of pharmaceutically acceptable carriers include water, saline, and phosphate-buffered saline.

The modified immune cells, immunomodulatory cells, iPSCs and/or iPSC-derived cells (or populations thereof) of the invention may be administered by any appropriate route. It may be desired to direct the compositions of the present invention (as described above) to the respiratory system of a subject. Efficient transmission of a therapeutic/prophylactic composition or medicament to the site of a disease or disorder in the respiratory tract may be achieved by oral or intra-nasal administration, for example, as aerosols (e.g. nasal sprays), or by catheters. Typically the modified immune cells, immunomodulatory cells, iPSCs and/or iPSC-derived cells (or populations thereof) of the invention are stable in clinically relevant nebulisers, inhalers (including metered dose inhalers), catheters and aerosols, etc.

In some embodiments the nose is a preferred production site for a therapeutic protein using modified immune cells, immunomodulatory cells, iPSCs and/or iPSC-derived cells (or populations thereof) of the invention for at least one of the following reasons: (i) extracellular barriers such as inflammatory cells and sputum are less pronounced in the nose; (ii) ease of cell administration; (iii) smaller quantities of cells required; and (iv) ethical considerations. Thus, nasal administration of modified immune cells, immunomodulatory cells, iPSCs and/or iPSC-derived cells (or populations thereof) of the invention may result in efficient (high-level) and long-lasting expression of the therapeutic transgene of interest. Accordingly, nasal administration of modified immune cells, immunomodulatory cells, iPSCs and/or iPSC-derived cells (or populations thereof) of the invention may be preferred.

Formulations for intra-nasal administration may be in the form of nasal droplets or a nasal spray. An intra-nasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

The aerosol formulation may take the form of a suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

The formulation of pharmaceutical aerosols is routine to those skilled in the art, see for example, Sciarra, J. in Remington's Pharmaceutical Sciences (supra). The agents may be formulated as solution aerosols or emulsions or. The aerosol may be delivered using any propellant system known to those skilled in the art. The aerosols may be applied to the upper respiratory tract, for example by nasal inhalation, or to the lower respiratory tract or to both. The part of the lung that the medicament is delivered to may be determined by the disorder. Compositions comprising modified immune cells, immunomodulatory cells, iPSCs and/or iPSC-derived cells (or populations thereof) of the invention, in particular where intranasal delivery is to be used, may comprise a humectant. This may help reduce or prevent drying of the mucus membrane and to prevent irritation of the membranes. Suitable humectants include, for instance, sorbitol, mineral oil, vegetable oil and glycerol; soothing agents; membrane conditioners; sweeteners; and combinations thereof. The compositions may comprise a surfactant. Suitable surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

In some cases after an initial administration a subsequent administration of modified immune cells, immunomodulatory cells, iPSCs and/or iPSC-derived cells (or populations thereof) of the invention may be performed. The administration may, for instance, be at least a week, two weeks, a month, two months, six months, a year or more after the initial administration. In some instances, modified immune cells, immunomodulatory cells, iPSCs and/or iPSC-derived cells (or populations thereof) of the invention may be administered at least once a week, once a fortnight, once a month, every two months, every six months, annually or at longer intervals. Preferably, administration is every six months, more preferably annually. The modified immune cells, immunomodulatory cells, iPSCs and/or iPSC-derived cells (or populations thereof) may, for instance, be administered at intervals dictated by when the effects of the previous administration are decreasing.

Any two or more types of modified immune cells, immunomodulatory cells, iPSCs and/or iPSC-derived cells (or populations thereof) may be administered separately, sequentially or simultaneously. Thus two types of modified immune cells, immunomodulatory cells, iPSCs and/or iPSC-derived cells (or populations thereof), where at least one type of modified immune cells, immunomodulatory cells, iPSCs and/or iPSC-derived cells (or populations thereof) is according to the invention, may be administered separately, simultaneously or sequentially and in particular two or more types of modified immune cells, immunomodulatory cells, iPSCs and/or iPSC-derived cells (or populations thereof) of the invention may be administered in such a manner. The two may be administered in the same or different compositions. In a preferred instance, the two types of modified immune cells, immunomodulatory cells, iPSCs and/or iPSC-derived cells (or populations thereof) may be delivered in the same composition.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264 (4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8 (5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M-A New Algorithm for Multiple Alignment of Highly Divergent Sequences, 20 (9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

A R N D C Q E G H I L K M F P S T W Y V
A 4
R −1 5
N −2 0 6
D −2 −2 1 6
C 0 −3 −3 −3 9
Q −1 1 0 0 −3 5
E −1 0 0 2 −4 2 5
G 0 −2 0 −1 −3 −2 −2 6
H −2 0 1 −1−3 0 0−2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −2 1 1
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### Sequence Information

### Key to Sequences

SEQ ID NO: 1 Exemplified hCEF promoter
SEQ ID NO: 2 Exemplified CMV promoter
SEQ ID NO: 3 Exemplified EF1a promoter
SEQ ID NO: 4 Exemplified CFTR transgene (soCFTR2)
SEQ ID NO: 5 Exemplified CFTR polypeptide
SEQ ID NO: 6 Exemplified Human GM-CSF transgene
SEQ ID NO: 7 Exemplified Human GM-CSF polypeptide
SEQ ID NO: 8 Exemplified Mouse GM-CSF transgene
SEQ ID NO: 9 Exemplified Mouse GM-CSF polypeptide
SEQ ID NO: 10 Exemplified SERPINA1 (AAT) transgene
SEQ ID NO: 11 Complementary strand to the exemplified SERPINA1 (AAT) transgene
SEQ ID NO: 12 Exemplified AAT polypeptide
SEQ ID NO: 13 Exemplified FVIII transgene (N6)
SEQ ID NO: 14 Exemplified FVIII transgene (V3)
SEQ ID NO: 15 Complementary strand to the exemplified FVIII transgene (N6)
SEQ ID NO: 16 Complementary strand to the exemplified FVIII transgene (V3)
SEQ ID NO: 17 Exemplified FVIII polypeptide (N6)
SEQ ID NO: 18 Exemplified FVIII polypeptide (V3)
SEQ ID NO: 19 Exemplified Human DCN (Decorin) transgene
SEQ ID NO: 20 Exemplified Human Decorin polypeptide
SEQ ID NO: 21 Exemplified Human TRIM72 transgene
SEQ ID NO: 22 Exemplified Human TRIM72 polypeptide
SEQ ID NO: 23 Exemplified Human ABACA3 (ABCA3) transgene
SEQ ID NO: 24 Exemplified Human ABCA3 polypeptide
SEQ ID NO: 25 Exemplified WPRE component (mWPRE)
SEQ ID NO: 26 Exemplified mifepristone-regulated promoter sequence
SEQ ID NO: 27 Exemplified nucleic acid sequence encoding a trans-activator for use with a mifepristone-regulated promoter

### Sequences

SEQ ID NO: 1 Exemplified hCEF promoter

| | |
|---|---|
| 1 | AGATCTGTTA CATAACTTAT GGTAAATGGC CTGCCTGGCT GACTGCCCAA TGACCCCTGC |
| 61 | CCAATGATGT CAATAATGAT GTATGTTCCC ATGTAATGCC AATAGGGACT TTCCATTGAT |
| 121 | GTCAATGGGT GGAGTATTTA TGGTAACTGC CCACTTGGCA GTACATCAAG TGTATCATAT |
| 181 | GCCAAGTATG CCCCCTATTG ATGTCAATGA TGGTAAATGG CCTGCCTGGC ATTATGCCCA |
| 241 | GTACATGACC TTATGGGACT TTCCTACTTG GCAGTACATC TATGTATTAG TCATTGCTAT |
| 301 | TACCATGGGA ATTCACTAGT GGAGAAGAGC ATGCTTGAGG GCTGAGTGCC CCTCAGTGGG |
| 361 | CAGAGAGCAC ATGGCCCACA GTCCCTGAGA AGTTGGGGGG AGGGGTGGGC AATTGAACTG |
| 421 | GTGCCTAGAG AAGGTGGGGC TTGGGTAAAC TGGGAAAGTG ATGTGGTGTA CTGGCTCCAC |
| 481 | CTTTTTCCCC AGGGTGGGGG AGAACCATAT ATAAGTGCAG TAGTCTCTGT GAACATTCAA |
| 541 | GCTTCTGCCT TCTCCCTCCT GTGAGTTTGC TAGC |

SEQ ID NO: 2 Exemplified CMV promoter
CCGCGGAGATCTCAATATTGGCCATTAGCCATATTATTCATTGGTTATATAGCATAAATCAATATTGGCT
ATTGGCCATTGCATACGTTGTATCTATATCATAATATGTACATTTATATTGGCTCATGTCCAATATGACC
GCCATGTTGGCATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATTGACGTCAATGGGT
GGAGTATTTACGGTAAACTGCCCACTTGGCAGTACATCAAGTGTATCATATGCCAAGTCCGCCCCCTATT
GACGTCAATGACGGTAAATGGCCCGCCTGGCATTATGCCCAGTACATGACCTTACGGGACTTTCCTACTT
GGCAGTACATCTACGTATTAGTCATCGCTATTACCATGGTGATGCGGTTTTGGCAGTACACCAATGGGCG
TGGATAGCGGTTTGACTCACGGGGATTTCCAAGTCTCCACCCCATTGACGTCAATGGGAGTTTGTTTTGG
CACCAAAATCAACGGGACTTTCCAAAATGTCGTAATAACCCCGCCCCGTTGACGCAAATGGGCGGTAGGC
GTGTACGGTGGGAGGTCTATATAAGCAGAGCTCGTTTAGTGAACCGTCAGATCACTAGAAGCTTTATTGC
GGTAGTTTATCACAGTTAAATTGCTAACGCAGTCAGTGCTTCTGACACAACAGTCTCGAACTTAAGCTGC
AGAAGTTGGTCGTGAGGCACTGGGCAGGCTAGC
SEQ ID NO: 3 Exemplified EF1a promoter
AGATCCATATCCGCGGCAATTTTAAAAGAAAGGGAGGAATAGGGGGACAGACTTCAGCAGAGAGACTAATTAATA
TAATAACAACACAATTAGAAATACAACATTTACAAACCAAAATTCAAAAAATTTTAAATTTTAGAGCCGCGGAGA
TCCCGTGAGGCTCCGGTGCCCGTCAGTGGGCAGAGCGCACATCGCCCACAGTCCCCGAGAAGTTGGGGGGAGGGG
TCGGCAATTGAACCGGTGCCTAGAGAAGGTGGCGCGGGGTAAACTGGGAAAGTGATGTCGTGTACTGGCTCCGCC
TTTTTCCCGAGGGTGGGGGAGAACCGTATATAAGTGCAGTAGTCGCCGTGAACGTTCTTTTTCGCAACGGGTTTG
CCGCCAGAACACAGGCTAGC
SEQ ID NO: 4 Exemplified CFTR transgene (soCFTR2)
ATGCAGAGAAGCCCTCTGGAGAAGGCCTCTGTGGTGAGCAAGCTGTTCTTCAGCTGGACCAGGCCCATCCTGAGG
AAGGGCTACAGGCAGAGACTGGAGCTGTCTGACATCTACCAGATCCCCTCTGTGGACTCTGCTGACAACCTGTCT
GAGAAGCTGGAGAGGGAGTGGGATAGAGAGCTGGCCAGCAAGAAGAACCCCAAGCTGATCAATGCCCTGAGGAGA
TGCTTCTTCTGGAGATTCATGTTCTATGGCATCTTCCTGTACCTGGGGGAAGTGACCAAGGCTGTGCAGCCTCTG
CTGCTGGGCAGAATCATTGCCAGCTATGACCCTGACAACAAGGAGGAGAGGAGCATTGCCATCTACCTGGGCATT
GGCCTGTGCCTGCTGTTCATTGTGAGGACCCTGCTGCTGCACCCTGCCATCTTTGGCCTGCACCACATTGGCATG
CAGATGAGGATTGCCATGTTCAGCCTGATCTACAAGAAAACCCTGAAGCTGTCCAGCAGAGTGCTGGACAAGATC
AGCATTGGCCAGCTGGTGAGCCTGCTGAGCAACAACCTGAACAAGTTTGATGAGGGCCTGGCCCTGGCCCACTTT
GTGTGGATTGCCCCTCTGCAGGTGGCCCTGCTGATGGGCCTGATTTGGGAGCTGCTGCAGGCCTCTGCCTTTTGT
GGCCTGGGCTTCCTGATTGTGCTGGCCCTGTTTCAGGCTGGCCTGGGCAGGATGATGATGAAGTACAGGGACCAG
AGGGCAGGCAAGATCAGTGAGAGGCTGGTGATCACCTCTGAGATGATTGAGAACATCCAGTCTGTGAAGGCCTAC
TGTTGGGAGGAAGCTATGGAGAAGATGATTGAAAACCTGAGGCAGACAGAGCTGAAGCTGACCAGGAAGGCTGCC
TATGTGAGATACTTCAACAGCTCTGCCTTCTTCTTCTCTGGCTTCTTTGTGGTGTTCCTGTCTGTGCTGCCCTAT
GCCCTGATCAAGGGGATCATCCTGAGAAAGATTTTCACCACCATCAGCTTCTGCATTGTGCTGAGGATGGCTGTG
ACCAGACAGTTCCCCTGGGCTGTGCAGACCTGGTATGACAGCCTGGGGGCCATCAACAAGATCCAGGACTTCCTG
CAGAAGCAGGAGTACAAGACCCTGGAGTACAACCTGACCACCACAGAAGTGGTGATGGAGAATGTGACAGCCTTC
TGGGAGGAGGGCTTTGGGGAGCTGTTTGAGAAGGCCAAGCAGAACAACAACAACAGAAAGACCAGCAATGGGGAT
GACTCCCTGTTCTTCTCCAACTTCTCCCTGCTGGGCACACCTGTGCTGAAGGACATCAACTTCAAGATTGAGAGG
GGGCAGCTGCTGGCTGTGGCTGGATCTACAGGGGCTGGCAAGACCAGCCTGCTGATGATGATCATGGGGGAGCTG
GAGCCTTCTGAGGGCAAGATCAAGCACTCTGGCAGGATCAGCTTTTGCAGCCAGTTCAGCTGGATCATGCCTGGC
ACCATCAAGGAGAACATCATCTTTGGAGTGAGCTATGATGAGTACAGATACAGGAGTGTGATCAAGGCCTGCCAG
CTGGAGGAGGACATCAGCAAGTTTGCTGAGAAGGACAACATTGTGCTGGGGGAGGGAGGCATTACACTGTCTGGG
GGCCAGAGAGCCAGAATCAGCCTGGCCAGGGCTGTGTACAAGGATGCTGACCTGTACCTGCTGGACTCCCCCTTT
GGCTACCTGGATGTGCTGACAGAGAAGGAGATTTTTGAGAGCTGTGTGTGCAAGCTGATGGCCAACAAGACCAGA
ATCCTGGTGACCAGCAAGATGGAGCACCTGAAGAAGGCTGACAAGATCCTGATCCTGCATGAGGGCAGCAGCTAC
TTCTATGGGACCTTCTCTGAGCTGCAGAACCTGCAGCCTGACTTCAGCTCTAAGCTGATGGGCTGTGACAGCTTT
GACCAGTTCTCTGCTGAGAGGAGGAACAGCATCCTGACAGAGACCCTGCACAGATTCAGCCTGGAGGGAGATGCC
CCTGTGAGCTGGACAGAGACCAAGAAGCAGAGCTTCAAGCAGACAGGGGAGTTTGGGGAGAAGAGGAAGAACTCC
ATCCTGAACCCCATCAACAGCATCAGGAAGTTCAGCATTGTGCAGAAAACCCCCCTGCAGATGAATGGCATTGAG
GAAGATTCTGATGAGCCCCTGGAGAGGAGACTGAGCCTGGTGCCTGATTCTGAGCAGGGAGAGGCCATCCTGCCT
AGGATCTCTGTGATCAGCACAGGCCCTACACTGCAGGCCAGAAGGAGGCAGTCTGTGCTGAACCTGATGACCCAC
TCTGTGAACCAGGGCCAGAACATCCACAGGAAAACCACAGCCTCCACCAGGAAAGTGAGCCTGGCCCCTCAGGCC
AATCTGACAGAGCTGGACATCTACAGCAGGAGGCTGTCTCAGGAGACAGGCCTGGAGATTTCTGAGGAGATCAAT
GAGGAGGACCTGAAAGAGTGCTTCTTTGATGACATGGAGAGCATCCCTGCTGTGACCACCTGGAACACCTACCTG
AGATACATCACAGTGCACAAGAGCCTGATCTTTGTGCTGATCTGGTGCCTGGTGATCTTCCTGGCTGAAGTGGCT
GCCTCTCTGGTGGTGCTGTGGCTGCTGGGAAACACCCCACTGCAGGACAAGGGCAACAGCACCCACAGCAGGAAC
AACAGCTATGCTGTGATCATCACCTCCACCTCCAGCTACTATGTGTTCTACATCTATGTGGGAGTGGCTGATACC
CTGCTGGCTATGGGCTTCTTTAGAGGCCTGCCCCTGGTGCACACACTGATCACAGTGAGCAAGATCCTCCACCAC
AAGATGCTGCACTCTGTGCTGCAGGCTCCTATGAGCACCCTGAATACCCTGAAGGCTGGGGGCATCCTGAACAGA
TTCTCCAAGGATATTGCCATCCTGGATGACCTGCTGCCTCTCACCATCTTTGACTTCATCCAGCTGCTGCTGATT
GTGATTGGGGCCATTGCTGTGGTGGCAGTGCTGCAGCCCTACATCTTTGTGGCCACAGTGCCTGTGATTGTGGCC
TTCATCATGCTGAGGGCCTACTTTCTGCAGACCTCCCAGCAGCTGAAGCAGCTGGAGTCTGAGGGCAGAAGCCCC
ATCTTCACCCACCTGGTGACAAGCCTGAAGGGCCTGTGGACCCTGAGAGCCTTTGGCAGGCAGCCCTACTTTGAG
ACCCTGTTCCACAAGGCCCTGAACCTGCACACAGCCAACTGGTTCCTCTACCTGTCCACCCTGAGATGGTTCCAG
ATGAGAATTGAGATGATCTTTGTCATCTTCTTCATTGCTGTGACCTTCATCAGCATTCTGACCACAGGAGAGGGA
GAGGGCAGAGTGGGCATTATCCTGACCCTGGCCATGAACATCATGAGCACACTGCAGTGGGCAGTGAACAGCAGC
ATTGATGTGGACAGCCTGATGAGGAGTGTGAGCAGAGTGTTCAAGTTCATTGATATGCCCACAGAGGGCAAGCCT
ACCAAGAGCACCAAGCCCTACAAGAATGGCCAGCTGAGCAAAGTGATGATCATTGAGAACAGCCATGTGAAGAAG
GATGATATCTGGCCCAGTGGAGGCCAGATGACAGTGAAGGACCTGACAGCCAAGTACACAGAGGGGGGCAATGCT
ATCCTGGAGAACATCTCCTTCAGCATCTCCCCTGGCCAGAGAGTGGGACTGCTGGGAAGAACAGGCTCTGGCAAG
TCTACCCTGCTGTCTGCCTTCCTGAGGCTGCTGAACACAGAGGGAGAGATCCAGATTGATGGAGTGTCCTGGGAC
AGCATCACACTGCAGCAGTGGAGGAAGGCCTTTGGTGTGATCCCCCAGAAAGTGTTCATCTTCAGTGGCACCTTC
AGGAAGAACCTGGACCCCTATGAGCAGTGGTCTGACCAGGAGATTTGGAAAGTGGCTGATGAAGTGGGCCTGAGA
AGTGTGATTGAGCAGTTCCCTGGCAAGCTGGACTTTGTCCTGGTGGATGGGGGCTGTGTGCTGAGCCATGGCCAC
AAGCAGCTGATGTGCCTGGCCAGATCAGTGCTGAGCAAGGCCAAGATCCTGCTGCTGGATGAGCCTTCTGCCCAC
CTGGATCCTGTGACCTACCAGATCATCAGGAGGACCCTCAAGCAGGCCTTTGCTGACTGCACAGTCATCCTGTGT
GAGCACAGGATTGAGGCCATGCTGGAGTGCCAGCAGTTCCTGGTGATTGAGGAGAACAAAGTGAGGCAGTATGAC
AGCATCCAGAAGCTGCTGAATGAGAGGAGCCTGTTCAGGCAGGCCATCAGCCCCTCTGATAGAGTGAAGCTGTTC
CCCCACAGGAACAGCTCCAAGTGCAAGAGCAAGCCCCAGATTGCTGCCCTGAAGGAGGAGACAGAGGAGGAAGTG
CAGGACACCAGGCTGTGA
SEQ ID NO: 5 Exemplified CFTR polypeptide
MQRSPLEKASVVSKLFFSWTRPILRKGYRQRLELSDIYQIPSVDSADNLSEKLEREWDRELASKKNPKLINALRR
CFFWRFMFYGIFLYLGEVTKAVQPLLLGRIIASYDPDNKEERSIAIYLGIGLCLLFIVRTLLLHPAIFGLHHIGM
QMRIAMFSLIYKKTLKLSSRVLDKISIGQLVSLLSNNLNKFDEGLALAHFVWIAPLQVALLMGLIWELLQASAFC
GLGFLIVLALFQAGLGRMMMKYRDQRAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAVTRQFPWAVQTWYDSLGAINKIQDEL
QKQEYKTLEYNLTTTEVVMENVTAFWEEGFGELFEKAKQNNNNRKTSNGDDSLFFSNFSLLGTPVLKDINFKIER
GQLLAVAGSTGAGKTSLLMMIMGELEPSEGKIKHSGRISFCSQFSWIMPGTIKENIIFGVSYDEYRYRSVIKACQ
LEEDISKFAEKDNIVLGEGGITLSGGQRARISLARAVYKDADLYLLDSPFGYLDVLTEKEIFESCVCKLMANKTR
ILVTSKMEHLKKADKILILHEGSSYFYGTFSELQNLQPDFSSKLMGCDSFDQFSAERRNSILTETLHRFSLEGDA
PVSWTETKKQSFKQTGEFGEKRKNSILNPINSIRKESIVQKTPLQMNGIEEDSDEPLERRLSLVPDSEQGEAILP
RISVISTGPTLQARRRQSVLNLMTHSVNQGQNIHRKTTASTRKVSLAPQANLTELDIYSRRLSQETGLEISEEIN
EEDLKECFFDDMESIPAVTTWNTYLRYITVHKSLIFVLIWCLVIFLAEVAASLVVLWLLGNTPLQDKGNSTHSRN
NSYAVIITSTSSYYVFYIYVGVADTLLAMGFFRGLPLVHTLITVSKILHHKMLHSVLQAPMSTLNTLKAGGILNR
FSKDIAILDDLLPLTIFDFIQLLLIVIGAIAVVAVLQPYIFVATVPVIVAFIMLRAYFLQTSQQLKQLESEGRSP
IFTHLVTSLKGLWTLRAFGRQPYFETLFHKALNLHTANWFLYLSTLRWFQMRIEMIFVIFFIAVTFISILTTGEG
EGRVGIILTLAMNIMSTLQWAVNSSIDVDSLMRSVSRVFKFIDMPTEGKPTKSTKPYKNGQLSKVMIIENSHVKK
DDIWPSGGQMTVKDLTAKYTEGGNAILENISFSISPGQRVGLLGRTGSGKSTLLSAFLRLLNTEGEIQIDGVSWD
SITLQQWRKAFGVIPQKVFIFSGTFRKNLDPYEQWSDQEIWKVADEVGLRSVIEQFPGKLDFVLVDGGCVLSHGH
KQLMCLARSVLSKAKILLLDEPSAHLDPVTYQIIRRTLKQAFADCTVILCEHRIEAMLECQQFLVIEENKVRQYD
SIQKLLNERSLFRQAISPSDRVKLFPHRNSSKCKSKPQIAALKEETEEEVQDTRL
SEQ ID NO: 6 Exemplified Human GM-CSF (CSF2) transgene
ATGTGGCTGCAGAGCCTGCTGCTCTTGGGCACTGTGGCCTGCAGCATCTCTGCACCCGCCCGCTCGCCCAGCCCC
AGCACGCAGCCCTGGGAGCATGTGAATGCCATCCAGGAGGCCCGGCGTCTCCTGAACCTGAGTAGAGACACTGCT
GCTGAGATGAATGAAACAGTAGAAGTCATCTCAGAAATGTTTGACCTCCAGGAGCCGACCTGCCTACAGACCCGC
CTGGAGCTGTACAAGCAGGGCCTGCGGGGCAGCCTCACCAAGCTCAAGGGCCCCTTGACCATGATGGCCAGCCAC
TACAAGCAGCACTGCCCTCCAACCCCGGAAACTTCCTGTGCAACCCAGATTATCACCTTTGAAAGTTTCAAAGAG
AACCTGAAGGACTTTCTGCTTGTCATCCCCTTTGACTGCTGGGAGCCAGTCCAGGAGTGA
Nucleic acid sequence encoding signal peptide is underlined.
SEQ ID NO: 7 Exemplified Human GM-CSF polypeptide
MWLQSLLLLGTVACSISAPARSPSPSTQPWEHVNAIQEARRLLNLSRDTAAEMNETVEVISEMFDLQEPTCLQTR
LELYKQGLRGSLTKLKGPLTMMASHYKQHCPPTPETSCATQIITFESFKENLKDELLVIPFDCWEPVQE
signal peptide is underlined.
SEQ ID NO: 8 Exemplified Mouse GM-CSF (CSF2) transgene
ATGTGGCTGCAGAACCTGCTGTTCCTGGGCATTGTGGTGTACAGCCTGTCTGCCCCTACAAGATCCCCTATCACA
GTGACCAGACCTTGGAAACATGTGGAAGCCATCAAAGAGGCCCTGAATCTGCTGGATGACATGCCTGTGACACTG
AATGAAGAGGTGGAAGTGGTGTCCAATGAGTTCAGCTTCAAGAAACTGACCTGTGTGCAGACCAGGCTGAAGATT
TTTGAGCAGGGCCTGAGAGGCAACTTCACCAAGCTGAAAGGGGCTCTGAACATGACAGCCAGCTACTACCAGACC
TACTGTCCTCCTACACCTGAGACAGACTGTGAAACCCAAGTGACCACCTATGCTGACTTCATTGACAGCCTCAAG
ACCTTCCTGACAGACATCCCCTTTGAGTGCAAGAAACCTGGCCAGAAGTGA
Nucleic acid sequence encoding signal peptide is underlined.
SEQ ID NO: 9 Exemplified Mouse GM-CSF polypeptide
MWLQNLLFLGIVVYSLSAPTRSPITVTRPWKHVEAIKEALNLLDDMPVTLNEEVEVVSNEFSFKKLTCVQTRLKI
FEQGLRGNFTKLKGALNMTASYYQTYCPPTPETDCETQVTTYADFIDSLKTELTDIPFECKKPGQK
signal peptide is underlined.
SEQ ID NO: 10 Exemplified SERPINA1 (AAT) transgene
ATGCCCAGCTCTGTGTCCTGGGGCATTCTGCTGCTGGCTGGCCTGTGCTGTCTGGTGCCTGTGTCCCTGG
CTGAGGACCCTCAGGGGGATGCTGCCCAGAAAACAGACACCTCCCACCATGACCAGGACCACCCCACCTT
CAACAAGATCACCCCCAACCTGGCAGAGTTTGCCTTCAGCCTGTACAGACAGCTGGCCCACCAGAGCAAC
AGCACCAACATCTTTTTCAGCCCTGTGTCCATTGCCACAGCCTTTGCCATGCTGAGCCTGGGCACCAAGG
CTGACACCCATGATGAGATCCTGGAAGGCCTGAACTTCAACCTGACAGAGATCCCTGAGGCCCAGATCCA
TGAGGGCTTCCAGGAACTGCTGAGAACCCTGAACCAGCCAGACAGCCAGCTGCAGCTGACAACAGGCAAT
GGGCTGTTCCTGTCTGAGGGCCTGAAGCTGGTGGACAAGTTTCTGGAAGATGTGAAGAAGCTGTACCACT
CTGAGGCCTTCACAGTGAACTTTGGGGACACAGAAGAGGCCAAGAAACAGATCAATGACTATGTGGAAAA
GGGCACCCAGGGCAAGATTGTGGACCTTGTGAAAGAGCTGGACAGGGACACTGTGTTTGCCCTTGTGAAC
TACATCTTCTTCAAGGGCAAGTGGGAGAGGCCCTTTGAAGTGAAGGACACTGAGGAAGAGGACTTCCATG
TGGACCAAGTGACCACAGTGAAGGTGCCAATGATGAAGAGACTGGGGATGTTCAATATCCAGCACTGCAA
GAAACTGAGCAGCTGGGTGCTGCTGATGAAGTACCTGGGCAATGCTACAGCCATATTCTTTCTGCCTGAT
GAGGGCAAGCTGCAGCACCTGGAAAATGAGCTGACCCATGACATCATCACCAAATTTCTGGAAAATGAGG
ACAGAAGATCTGCCAGCCTGCATCTGCCCAAGCTGAGCATCACAGGCACATATGACCTGAAGTCTGTGCT
GGGACAGCTGGGAATCACCAAGGTGTTCAGCAATGGGGCAGACCTGAGTGGAGTGACAGAGGAAGCCCCT
CTGAAGCTGTCCAAGGCTGTGCACAAGGCAGTGCTGACCATTGATGAGAAGGGCACAGAGGCTGCTGGGG
CCATGTTTCTGGAAGCCATCCCCATGTCCATCCCCCCAGAAGTGAAGTTCAACAAGCCCTTTGTGTTCCT
GATGATTGAGCAGAACACCAAGAGCCCCCTGTTCATGGGCAAGGTTGTGAACCCCACCCAGAAATGA
Nucleic acid sequence encoding signal peptide is underlined.
SEQ ID NO: 11 Complementary strand to the exemplified SERPINA1 (AAT) transgene
TACGGGTCGAGACACAGGACCCCGTAAGACGACGACCGACCGGACACGACAGACCACGGACACAGGGACC
GACTCCTGGGAGTCCCCCTACGACGGGTCTTTTGTCTGTGGAGGGTGGTACTGGTCCTGGTGGGGTGGAA
GTTGTTCTAGTGGGGGTTGGACCGTCTCAAACGGAAGTCGGACATGTCTGTCGACCGGGTGGTCTCGTTG
TCGTGGTTGTAGAAAAAGTCGGGACACAGGTAACGGTGTCGGAAACGGTACGACTCGGACCCGTGGTTCC
GACTGTGGGTACTACTCTAGGACCTTCCGGACTTGAAGTTGGACTGTCTCTAGGGACTCCGGGTCTAGGT
ACTCCCGAAGGTCCTTGACGACTCTTGGGACTTGGTCGGTCTGTCGGTCGACGTCGACTGTTGTCCGTTA
CCCGACAAGGACAGACTCCCGGACTTCGACCACCTGTTCAAAGACCTTCTACACTTCTTCGACATGGTGA
GACTCCGGAAGTGTCACTTGAAACCCCTGTGTCTTCTCCGGTTCTTTGTCTAGTTACTGATACACCTTTT
CCCGTGGGTCCCGTTCTAACACCTGGAACACTTTCTCGACCTGTCCCTGTGACACAAACGGGAACACTTG
ATGTAGAAGAAGTTCCCGTTCACCCTCTCCGGGAAACTTCACTTCCTGTGACTCCTTCTCCTGAAGGTAC
ACCTGGTTCACTGGTGTCACTTCCACGGTTACTACTTCTCTGACCCCTACAAGTTATAGGTCGTGACGTT
CTTTGACTCGTCGACCCACGACGACTACTTCATGGACCCGTTACGATGTCGGTATAAGAAAGACGGACTA
CTCCCGTTCGACGTCGTGGACCTTTTACTCGACTGGGTACTGTAGTAGTGGTTTAAAGACCTTTTACTCC
TGTCTTCTAGACGGTCGGACGTAGACGGGTTCGACTCGTAGTGTCCGTGTATACTGGACTTCAGACACGA
CCCTGTCGACCCTTAGTGGTTCCACAAGTCGTTACCCCGTCTGGACTCACCTCACTGTCTCCTTCGGGGA
GACTTCGACAGGTTCCGACACGTGTTCCGTCACGACTGGTAACTACTCTTCCCGTGTCTCCGACGACCCC
GGTACAAAGACCTTCGGTAGGGGTACAGGTAGGGGGGTCTTCACTTCAAGTTGTTCGGGAAACACAAGGA
CTACTAACTCGTCTTGTGGTTCTCGGGGGACAAGTACCCGTTCCAACACTTGGGGTGGGTCTTTACT
SEQ ID NO: 12 Exemplified AAT polypeptide
MPSSVSWGILLLAGLCCLVPVSLAEDPQGDAAQKTDTSHHDQDHPTFAEDPQGDAAQKTDTSHHDQDHPTF
NKITPNLAEFAFSLYRQLAHQSNSTNIFFSPVSIATAFAMLSLGTKADTHDEILEGLNFNLTEIPEAQIHE
GFQELLRTLNQPDSQLQLTTGNGLFLSEGLKLVDKFLEDVKKLYHSEAFTVNFGDTEEAKKQINDYVEKGT
QGKIVDLVKELDRDTVFALVNYIFFKGKWERPFEVKDTEEEDFHVDQVTTVKVPMMKRLGMFNIQHCKKLS
SWVLLMKYLGNATAIFFLPDEGKLQHLENELTHDIITKFLENEDRRSASLHLPKLSITGTYDLKSVLGQLG
ITKVFSNGADLSGVTEEAPLKLSKAVHKAVLTIDEKGTEAAGAMFLEAIPMSIPPEVKENKPFVELMIEQN
TKSPLFMGKVVNPTQK
signal peptide is underlined.
SEQ ID NO: 13 Exemplified FVIII transgene (N6)
ATGCAGATTGAGCTGAGCACCTGCTTCTTCCTGTGCCTGCTGAGGTTCTGCTTCTCTGCCACCAGGAGAT
ACTACCTGGGGGCTGTGGAGCTGAGCTGGGACTACATGCAGTCTGACCTGGGGGAGCTGCCTGTGGATGC
CAGGTTCCCCCCCAGAGTGCCCAAGAGCTTCCCCTTCAACACCTCTGTGGTGTACAAGAAGACCCTGTTT
GTGGAGTTCACTGACCACCTGTTCAACATTGCCAAGCCCAGGCCCCCCTGGATGGGCCTGCTGGGCCCCA
CCATCCAGGCTGAGGTGTATGACACTGTGGTGATCACCCTGAAGAACATGGCCAGCCACCCTGTGAGCCT
GCATGCTGTGGGGGTGAGCTACTGGAAGGCCTCTGAGGGGGCTGAGTATGATGACCAGACCAGCCAGAGG
GAGAAGGAGGATGACAAGGTGTTCCCTGGGGGCAGCCACACCTATGTGTGGCAGGTGCTGAAGGAGAATG
GCCCCATGGCCTCTGACCCCCTGTGCCTGACCTACAGCTACCTGAGCCATGTGGACCTGGTGAAGGACCT
GAACTCTGGCCTGATTGGGGCCCTGCTGGTGTGCAGGGAGGGCAGCCTGGCCAAGGAGAAGACCCAGACC
CTGCACAAGTTCATCCTGCTGTTTGCTGTGTTTGATGAGGGCAAGAGCTGGCACTCTGAAACCAAGAACA
GCCTGATGCAGGACAGGGATGCTGCCTCTGCCAGGGCCTGGCCCAAGATGCACACTGTGAATGGCTATGT
GAACAGGAGCCTGCCTGGCCTGATTGGCTGCCACAGGAAGTCTGTGTACTGGCATGTGATTGGCATGGGC
ACCACCCCTGAGGTGCACAGCATCTTCCTGGAGGGCCACACCTTCCTGGTCAGGAACCACAGGCAGGCCA
GCCTGGAGATCAGCCCCATCACCTTCCTGACTGCCCAGACCCTGCTGATGGACCTGGGCCAGTTCCTGCT
GTTCTGCCACATCAGCAGCCACCAGCATGATGGCATGGAGGCCTATGTGAAGGTGGACAGCTGCCCTGAG
GAGCCCCAGCTGAGGATGAAGAACAATGAGGAGGCTGAGGACTATGATGATGACCTGACTGACTCTGAGA
TGGATGTGGTGAGGTTTGATGATGACAACAGCCCCAGCTTCATCCAGATCAGGTCTGTGGCCAAGAAGCA
CCCCAAGACCTGGGTGCACTACATTGCTGCTGAGGAGGAGGACTGGGACTATGCCCCCCTGGTGCTGGCC
CCTGATGACAGGAGCTACAAGAGCCAGTACCTGAACAATGGCCCCCAGAGGATTGGCAGGAAGTACAAGA
AGGTCAGGTTCATGGCCTACACTGATGAAACCTTCAAGACCAGGGAGGCCATCCAGCATGAGTCTGGCAT
CCTGGGCCCCCTGCTGTATGGGGAGGTGGGGGACACCCTGCTGATCATCTTCAAGAACCAGGCCAGCAGG
CCCTACAACATCTACCCCCATGGCATCACTGATGTGAGGCCCCTGTACAGCAGGAGGCTGCCCAAGGGGG
TGAAGCACCTGAAGGACTTCCCCATCCTGCCTGGGGAGATCTTCAAGTACAAGTGGACTGTGACTGTGGA
GGATGGCCCCACCAAGTCTGACCCCAGGTGCCTGACCAGATACTACAGCAGCTTTGTGAACATGGAGAGG
GACCTGGCCTCTGGCCTGATTGGCCCCCTGCTGATCTGCTACAAGGAGTCTGTGGACCAGAGGGGCAACC
AGATCATGTCTGACAAGAGGAATGTGATCCTGTTCTCTGTGTTTGATGAGAACAGGAGCTGGTACCTGAC
TGAGAACATCCAGAGGTTCCTGCCCAACCCTGCTGGGGTGCAGCTGGAGGACCCTGAGTTCCAGGCCAGC
AACATCATGCACAGCATCAATGGCTATGTGTTTGACAGCCTGCAGCTGTCTGTGTGCCTGCATGAGGTGG
CCTACTGGTACATCCTGAGCATTGGGGCCCAGACTGACTTCCTGTCTGTGTTCTTCTCTGGCTACACCTT
CAAGCACAAGATGGTGTATGAGGACACCCTGACCCTGTTCCCCTTCTCTGGGGAGACTGTGTTCATGAGC
ATGGAGAACCCTGGCCTGTGGATTCTGGGCTGCCACAACTCTGACTTCAGGAACAGGGGCATGACTGCCC
TGCTGAAAGTCTCCAGCTGTGACAAGAACACTGGGGACTACTATGAGGACAGCTATGAGGACATCTCTGC
CTACCTGCTGAGCAAGAACAATGCCATTGAGCCCAGGAGCTTCAGCCAGAACAGCAGGCACCCCAGCACC
AGGCAGAAGCAGTTCAATGCCACCACCATCCCTGAGAATGACATAGAGAAGACAGACCCATGGTTTGCCC
ACCGGACCCCCATGCCCAAGATCCAGAATGTGAGCAGCTCTGACCTGCTGATGCTGCTGAGGCAGAGCCC
CACCCCCCATGGCCTGAGCCTGTCTGACCTGCAGGAGGCCAAGTATGAAACCTTCTCTGATGACCCCAGC
CCTGGGGCCATTGACAGCAACAACAGCCTGTCTGAGATGACCCACTTCAGGCCCCAGCTGCACCACTCTG
GGGACATGGTGTTCACCCCTGAGTCTGGCCTGCAGCTGAGGCTGAATGAGAAGCTGGGCACCACTGCTGC
CACTGAGCTGAAGAAGCTGGACTTCAAAGTCTCCAGCACCAGCAACAACCTGATCAGCACCATCCCCTCT
GACAACCTGGCTGCTGGCACTGACAACACCAGCAGCCTGGGCCCCCCCAGCATGCCTGTGCACTATGACA
GCCAGCTGGACACCACCCTGTTTGGCAAGAAGAGCAGCCCCCTGACTGAGTCTGGGGGCCCCCTGAGCCT
GTCTGAGGAGAACAATGACAGCAAGCTGCTGGAGTCTGGCCTGATGAACAGCCAGGAGAGCAGCTGGGGC
AAGAATGTGAGCAGCAGGGAGATCACCAGGACCACCCTGCAGTCTGACCAGGAGGAGATTGACTATGATG
ACACCATCTCTGTGGAGATGAAGAAGGAGGACTTTGACATCTACGACGAGGACGAGAACCAGAGCCCCAG
GAGCTTCCAGAAGAAGACCAGGCACTACTTCATTGCTGCTGTGGAGAGGCTGTGGGACTATGGCATGAGC
AGCAGCCCCCATGTGCTGAGGAACAGGGCCCAGTCTGGCTCTGTGCCCCAGTTCAAGAAGGTGGTGTTCC
AGGAGTTCACTGATGGCAGCTTCACCCAGCCCCTGTACAGAGGGGAGCTGAATGAGCACCTGGGCCTGCT
GGGCCCCTACATCAGGGCTGAGGTGGAGGACAACATCATGGTGACCTTCAGGAACCAGGCCAGCAGGCCC
TACAGCTTCTACAGCAGCCTGATCAGCTATGAGGAGGACCAGAGGCAGGGGGCTGAGCCCAGGAAGAACT
TTGTGAAGCCCAATGAAACCAAGACCTACTTCTGGAAGGTGCAGCACCACATGGCCCCCACCAAGGATGA
GTTTGACTGCAAGGCCTGGGCCTACTTCTCTGATGTGGACCTGGAGAAGGATGTGCACTCTGGCCTGATT
GGCCCCCTGCTGGTGTGCCACACCAACACCCTGAACCCTGCCCATGGCAGGCAGGTGACTGTGCAGGAGT
TTGCCCTGTTCTTCACCATCTTTGATGAAACCAAGAGCTGGTACTTCACTGAGAACATGGAGAGGAACTG
CAGGGCCCCCTGCAACATCCAGATGGAGGACCCCACCTTCAAGGAGAACTACAGGTTCCATGCCATCAAT
GGCTACATCATGGACACCCTGCCTGGCCTGGTGATGGCCCAGGACCAGAGGATCAGGTGGTACCTGCTGA
GCATGGGCAGCAATGAGAACATCCACAGCATCCACTTCTCTGGCCATGTGTTCACTGTGAGGAAGAAGGA
GGAGTACAAGATGGCCCTGTACAACCTGTACCCTGGGGTGTTTGAGACTGTGGAGATGCTGCCCAGCAAG
GCTGGCATCTGGAGGGTGGAGTGCCTGATTGGGGAGCACCTGCATGCTGGCATGAGCACCCTGTTCCTGG
TGTACAGCAACAAGTGCCAGACCCCCCTGGGCATGGCCTCTGGCCACATCAGGGACTTCCAGATCACTGC
CTCTGGCCAGTATGGCCAGTGGGCCCCCAAGCTGGCCAGGCTGCACTACTCTGGCAGCATCAATGCCTGG
AGCACCAAGGAGCCCTTCAGCTGGATCAAGGTGGACCTGCTGGCCCCCATGATCATCCATGGCATCAAGA
CCCAGGGGGCCAGGCAGAAGTTCAGCAGCCTGTACATCAGCCAGTTCATCATCATGTACAGCCTGGATGG
CAAGAAGTGGCAGACCTACAGGGGCAACAGCACTGGCACCCTGATGGTGTTCTTTGGCAATGTGGACAGC
TCTGGCATCAAGCACAACATCTTCAACCCCCCCATCATTGCCAGATACATCAGGCTGCACCCCACCCACT
ACAGCATCAGGAGCACCCTGAGGATGGAGCTGATGGGCTGTGACCTGAACAGCTGCAGCATGCCCCTGGG
CATGGAGAGCAAGGCCATCTCTGATGCCCAGATCACTGCCAGCAGCTACTTCACCAACATGTTTGCCACC
TGGAGCCCCAGCAAGGCCAGGCTGCACCTGCAGGGCAGGAGCAATGCCTGGAGGCCCCAGGTCAACAACC
CCAAGGAGTGGCTGCAGGTGGACTTCCAGAAGACCATGAAGGTGACTGGGGTGACCACCCAGGGGGTGAA
GAGCCTGCTGACCAGCATGTATGTGAAGGAGTTCCTGATCAGCAGCAGCCAGGATGGCCACCAGTGGACC
CTGTTCTTCCAGAATGGCAAGGTGAAGGTGTTCCAGGGCAACCAGGACAGCTTCACCCCTGTGGTGAACA
GCCTGGACCCCCCCCTGCTGACCAGATACCTGAGGATTCACCCCCAGAGCTGGGTGCACCAGATTGCCCT
GAGGATGGAGGTGCTGGGCTGTGAGGCCCAGGACCTGTACTGA
Nucleic acid sequence encoding signal peptide is underlined.
SEQ ID NO: 14 Exemplified FVIII transgene (V3)
ATGCAGATTGAGCTGAGCACCTGCTTCTTCCTGTGCCTGCTGAGGTTCTGCTTCTCTGCCACCAGGAGAT
ACTACCTGGGGGCTGTGGAGCTGAGCTGGGACTACATGCAGTCTGACCTGGGGGAGCTGCCTGTGGATGC
CAGGTTCCCCCCCAGAGTGCCCAAGAGCTTCCCCTTCAACACCTCTGTGGTGTACAAGAAGACCCTGTTT
GTGGAGTTCACTGACCACCTGTTCAACATTGCCAAGCCCAGGCCCCCCTGGATGGGCCTGCTGGGCCCCA
CCATCCAGGCTGAGGTGTATGACACTGTGGTGATCACCCTGAAGAACATGGCCAGCCACCCTGTGAGCCT
GCATGCTGTGGGGGTGAGCTACTGGAAGGCCTCTGAGGGGGCTGAGTATGATGACCAGACCAGCCAGAGG
GAGAAGGAGGATGACAAGGTGTTCCCTGGGGGCAGCCACACCTATGTGTGGCAGGTGCTGAAGGAGAATG
GCCCCATGGCCTCTGACCCCCTGTGCCTGACCTACAGCTACCTGAGCCATGTGGACCTGGTGAAGGACCT
GAACTCTGGCCTGATTGGGGCCCTGCTGGTGTGCAGGGAGGGCAGCCTGGCCAAGGAGAAGACCCAGACC
CTGCACAAGTTCATCCTGCTGTTTGCTGTGTTTGATGAGGGCAAGAGCTGGCACTCTGAAACCAAGAACA
GCCTGATGCAGGACAGGGATGCTGCCTCTGCCAGGGCCTGGCCCAAGATGCACACTGTGAATGGCTATGT
GAACAGGAGCCTGCCTGGCCTGATTGGCTGCCACAGGAAGTCTGTGTACTGGCATGTGATTGGCATGGGC
ACCACCCCTGAGGTGCACAGCATCTTCCTGGAGGGCCACACCTTCCTGGTCAGGAACCACAGGCAGGCCA
GCCTGGAGATCAGCCCCATCACCTTCCTGACTGCCCAGACCCTGCTGATGGACCTGGGCCAGTTCCTGCT
GTTCTGCCACATCAGCAGCCACCAGCATGATGGCATGGAGGCCTATGTGAAGGTGGACAGCTGCCCTGAG
GAGCCCCAGCTGAGGATGAAGAACAATGAGGAGGCTGAGGACTATGATGATGACCTGACTGACTCTGAGA
TGGATGTGGTGAGGTTTGATGATGACAACAGCCCCAGCTTCATCCAGATCAGGTCTGTGGCCAAGAAGCA
CCCCAAGACCTGGGTGCACTACATTGCTGCTGAGGAGGAGGACTGGGACTATGCCCCCCTGGTGCTGGCC
CCTGATGACAGGAGCTACAAGAGCCAGTACCTGAACAATGGCCCCCAGAGGATTGGCAGGAAGTACAAGA
AGGTCAGGTTCATGGCCTACACTGATGAAACCTTCAAGACCAGGGAGGCCATCCAGCATGAGTCTGGCAT
CCTGGGCCCCCTGCTGTATGGGGAGGTGGGGGACACCCTGCTGATCATCTTCAAGAACCAGGCCAGCAGG
CCCTACAACATCTACCCCCATGGCATCACTGATGTGAGGCCCCTGTACAGCAGGAGGCTGCCCAAGGGGG
TGAAGCACCTGAAGGACTTCCCCATCCTGCCTGGGGAGATCTTCAAGTACAAGTGGACTGTGACTGTGGA
GGATGGCCCCACCAAGTCTGACCCCAGGTGCCTGACCAGATACTACAGCAGCTTTGTGAACATGGAGAGG
GACCTGGCCTCTGGCCTGATTGGCCCCCTGCTGATCTGCTACAAGGAGTCTGTGGACCAGAGGGGCAACC
AGATCATGTCTGACAAGAGGAATGTGATCCTGTTCTCTGTGTTTGATGAGAACAGGAGCTGGTACCTGAC
TGAGAACATCCAGAGGTTCCTGCCCAACCCTGCTGGGGTGCAGCTGGAGGACCCTGAGTTCCAGGCCAGC
AACATCATGCACAGCATCAATGGCTATGTGTTTGACAGCCTGCAGCTGTCTGTGTGCCTGCATGAGGTGG
CCTACTGGTACATCCTGAGCATTGGGGCCCAGACTGACTTCCTGTCTGTGTTCTTCTCTGGCTACACCTT
CAAGCACAAGATGGTGTATGAGGACACCCTGACCCTGTTCCCCTTCTCTGGGGAGACTGTGTTCATGAGC
ATGGAGAACCCTGGCCTGTGGATTCTGGGCTGCCACAACTCTGACTTCAGGAACAGGGGCATGACTGCCC
TGCTGAAAGTCTCCAGCTGTGACAAGAACACTGGGGACTACTATGAGGACAGCTATGAGGACATCTCTGC
CTACCTGCTGAGCAAGAACAATGCCATTGAGCCCAGGAGCTTCAGCCAGAATGCCACTAATGTGTCTAAC
AACAGCAACACCAGCAATGACAGCAATGTGTCTCCCCCAGTGCTGAAGAGGCACCAGAGGGAGATCACCA
GGACCACCCTGCAGTCTGACCAGGAGGAGATTGACTATGATGACACCATCTCTGTGGAGATGAAGAAGGA
GGACTTTGACATCTACGACGAGGACGAGAACCAGAGCCCCAGGAGCTTCCAGAAGAAGACCAGGCACTAC
TTCATTGCTGCTGTGGAGAGGCTGTGGGACTATGGCATGAGCAGCAGCCCCCATGTGCTGAGGAACAGGG
CCCAGTCTGGCTCTGTGCCCCAGTTCAAGAAGGTGGTGTTCCAGGAGTTCACTGATGGCAGCTTCACCCA
GCCCCTGTACAGAGGGGAGCTGAATGAGCACCTGGGCCTGCTGGGCCCCTACATCAGGGCTGAGGTGGAG
GACAACATCATGGTGACCTTCAGGAACCAGGCCAGCAGGCCCTACAGCTTCTACAGCAGCCTGATCAGCT
ATGAGGAGGACCAGAGGCAGGGGGCTGAGCCCAGGAAGAACTTTGTGAAGCCCAATGAAACCAAGACCTA
CTTCTGGAAGGTGCAGCACCACATGGCCCCCACCAAGGATGAGTTTGACTGCAAGGCCTGGGCCTACTTC
TCTGATGTGGACCTGGAGAAGGATGTGCACTCTGGCCTGATTGGCCCCCTGCTGGTGTGCCACACCAACA
CCCTGAACCCTGCCCATGGCAGGCAGGTGACTGTGCAGGAGTTTGCCCTGTTCTTCACCATCTTTGATGA
AACCAAGAGCTGGTACTTCACTGAGAACATGGAGAGGAACTGCAGGGCCCCCTGCAACATCCAGATGGAG
GACCCCACCTTCAAGGAGAACTACAGGTTCCATGCCATCAATGGCTACATCATGGACACCCTGCCTGGCC
TGGTGATGGCCCAGGACCAGAGGATCAGGTGGTACCTGCTGAGCATGGGCAGCAATGAGAACATCCACAG
CATCCACTTCTCTGGCCATGTGTTCACTGTGAGGAAGAAGGAGGAGTACAAGATGGCCCTGTACAACCTG
TACCCTGGGGTGTTTGAGACTGTGGAGATGCTGCCCAGCAAGGCTGGCATCTGGAGGGTGGAGTGCCTGA
TTGGGGAGCACCTGCATGCTGGCATGAGCACCCTGTTCCTGGTGTACAGCAACAAGTGCCAGACCCCCCT
GGGCATGGCCTCTGGCCACATCAGGGACTTCCAGATCACTGCCTCTGGCCAGTATGGCCAGTGGGCCCCC
AAGCTGGCCAGGCTGCACTACTCTGGCAGCATCAATGCCTGGAGCACCAAGGAGCCCTTCAGCTGGATCA
AGGTGGACCTGCTGGCCCCCATGATCATCCATGGCATCAAGACCCAGGGGGCCAGGCAGAAGTTCAGCAG
CCTGTACATCAGCCAGTTCATCATCATGTACAGCCTGGATGGCAAGAAGTGGCAGACCTACAGGGGCAAC
AGCACTGGCACCCTGATGGTGTTCTTTGGCAATGTGGACAGCTCTGGCATCAAGCACAACATCTTCAACC
CCCCCATCATTGCCAGATACATCAGGCTGCACCCCACCCACTACAGCATCAGGAGCACCCTGAGGATGGA
GCTGATGGGCTGTGACCTGAACAGCTGCAGCATGCCCCTGGGCATGGAGAGCAAGGCCATCTCTGATGCC
CAGATCACTGCCAGCAGCTACTTCACCAACATGTTTGCCACCTGGAGCCCCAGCAAGGCCAGGCTGCACC
TGCAGGGCAGGAGCAATGCCTGGAGGCCCCAGGTCAACAACCCCAAGGAGTGGCTGCAGGTGGACTTCCA
GAAGACCATGAAGGTGACTGGGGTGACCACCCAGGGGGTGAAGAGCCTGCTGACCAGCATGTATGTGAAG
GAGTTCCTGATCAGCAGCAGCCAGGATGGCCACCAGTGGACCCTGTTCTTCCAGAATGGCAAGGTGAAGG
TGTTCCAGGGCAACCAGGACAGCTTCACCCCTGTGGTGAACAGCCTGGACCCCCCCCTGCTGACCAGATA
CCTGAGGATTCACCCCCAGAGCTGGGTGCACCAGATTGCCCTGAGGATGGAGGTGCTGGGCTGTGAGGCC
CAGGACCTGTACTGA
Nucleic acid sequence encoding signal peptide is underlined.
SEQ ID NO: 15 Complementary strand to the exemplified FVIII transgene (N6)
TACGTCTAACTCGACTCGTGGACGAAGAAGGACACGGACGACTCCAAGACGAAGAGACGGTGGTCCTCTA
TGATGGACCCCCGACACCTCGACTCGACCCTGATGTACGTCAGACTGGACCCCCTCGACGGACACCTACG
GTCCAAGGGGGGGTCTCACGGGTTCTCGAAGGGGAAGTTGTGGAGACACCACATGTTCTTCTGGGACAAA
CACCTCAAGTGACTGGTGGACAAGTTGTAACGGTTCGGGTCCGGGGGGACCTACCCGGACGACCCGGGGT
GGTAGGTCCGACTCCACATACTGTGACACCACTAGTGGGACTTCTTGTACCGGTCGGTGGGACACTCGGA
CGTACGACACCCCCACTCGATGACCTTCCGGAGACTCCCCCGACTCATACTACTGGTCTGGTCGGTCTCC
CTCTTCCTCCTACTGTTCCACAAGGGACCCCCGTCGGTGTGGATACACACCGTCCACGACTTCCTCTTAC
CGGGGTACCGGAGACTGGGGGACACGGACTGGATGTCGATGGACTCGGTACACCTGGACCACTTCCTGGA
CTTGAGACCGGACTAACCCCGGGACGACCACACGTCCCTCCCGTCGGACCGGTTCCTCTTCTGGGTCTGG
GACGTGTTCAAGTAGGACGACAAACGACACAAACTACTCCCGTTCTCGACCGTGAGACTTTGGTTCTTGT
CGGACTACGTCCTGTCCCTACGACGGAGACGGTCCCGGACCGGGTTCTACGTGTGACACTTACCGATACA
CTTGTCCTCGGACGGACCGGACTAACCGACGGTGTCCTTCAGACACATGACCGTACACTAACCGTACCCG
TGGTGGGGACTCCACGTGTCGTAGAAGGACCTCCCGGTGTGGAAGGACCAGTCCTTGGTGTCCGTCCGGT
CGGACCTCTAGTCGGGGTAGTGGAAGGACTGACGGGTCTGGGACGACTACCTGGACCCGGTCAAGGACGA
CAAGACGGTGTAGTCGTCGGTGGTCGTACTACCGTACCTCCGGATACACTTCCACCTGTCGACGGGACTC
CTCGGGGTCGACTCCTACTTCTTGTTACTCCTCCGACTCCTGATACTACTACTGGACTGACTGAGACTCT
ACCTACACCACTCCAAACTACTACTGTTGTCGGGGTCGAAGTAGGTCTAGTCCAGACACCGGTTCTTCGT
GGGGTTCTGGACCCACGTGATGTAACGACGACTCCTCCTCCTGACCCTGATACGGGGGGACCACGACCGG
GGACTACTGTCCTCGATGTTCTCGGTCATGGACTTGTTACCGGGGGTCTCCTAACCGTCCTTCATGTTCT
TCCAGTCCAAGTACCGGATGTGACTACTTTGGAAGTTCTGGTCCCTCCGGTAGGTCGTACTCAGACCGTA
GGACCCGGGGGACGACATACCCCTCCACCCCCTGTGGGACGACTAGTAGAAGTTCTTGGTCCGGTCGTCC
GGGATGTTGTAGATGGGGGTACCGTAGTGACTACACTCCGGGGACATGTCGTCCTCCGACGGGTTCCCCC
ACTTCGTGGACTTCCTGAAGGGGTAGGACGGACCCCTCTAGAAGTTCATGTTCACCTGACACTGACACCT
CCTACCGGGGTGGTTCAGACTGGGGTCCACGGACTGGTCTATGATGTCGTCGAAACACTTGTACCTCTCC
CTGGACCGGAGACCGGACTAACCGGGGGACGACTAGACGATGTTCCTCAGACACCTGGTCTCCCCGTTGG
TCTAGTACAGACTGTTCTCCTTACACTAGGACAAGAGACACAAACTACTCTTGTCCTCGACCATGGACTG
ACTCTTGTAGGTCTCCAAGGACGGGTTGGGACGACCCCACGTCGACCTCCTGGGACTCAAGGTCCGGTCG
TTGTAGTACGTGTCGTAGTTACCGATACACAAACTGTCGGACGTCGACAGACACACGGACGTACTCCACC
GGATGACCATGTAGGACTCGTAACCCCGGGTCTGACTGAAGGACAGACACAAGAAGAGACCGATGTGGAA
GTTCGTGTTCTACCACATACTCCTGTGGGACTGGGACAAGGGGAAGAGACCCCTCTGACACAAGTACTCG
TACCTCTTGGGACCGGACACCTAAGACCCGACGGTGTTGAGACTGAAGTCCTTGTCCCCGTACTGACGGG
ACGACTTTCAGAGGTCGACACTGTTCTTGTGACCCCTGATGATACTCCTGTCGATACTCCTGTAGAGACG
GATGGACGACTCGTTCTTGTTACGGTAACTCGGGTCCTCGAAGTCGGTCTTGTCGTCCGTGGGGTCGTGG
TCCGTCTTCGTCAAGTTACGGTGGTGGTAGGGACTCTTACTGTATCTCTTCTGTCTGGGTACCAAACGGG
TGGCCTGGGGGTACGGGTTCTAGGTCTTACACTCGTCGAGACTGGACGACTACGACGACTCCGTCTCGGG
GTGGGGGGTACCGGACTCGGACAGACTGGACGTCCTCCGGTTCATACTTTGGAAGAGACTACTGGGGTCG
GGACCCCGGTAACTGTCGTTGTTGTCGGACAGACTCTACTGGGTGAAGTCCGGGGTCGACGTGGTGAGAC
CCCTGTACCACAAGTGGGGACTCAGACCGGACGTCGACTCCGACTTACTCTTCGACCCGTGGTGACGACG
GTGACTCGACTTCTTCGACCTGAAGTTTCAGAGGTCGTGGTCGTTGTTGGACTAGTCGTGGTAGGGGAGA
CTGTTGGACCGACGACCGTGACTGTTGTGGTCGTCGGACCCGGGGGGGTCGTACGGACACGTGATACTGT
CGGTCGACCTGTGGTGGGACAAACCGTTCTTCTCGTCGGGGGACTGACTCAGACCCCCGGGGGACTCGGA
CAGACTCCTCTTGTTACTGTCGTTCGACGACCTCAGACCGGACTACTTGTCGGTCCTCTCGTCGACCCCG
TTCTTACACTCGTCGTCCCTCTAGTGGTCCTGGTGGGACGTCAGACTGGTCCTCCTCTAACTGATACTAC
TGTGGTAGAGACACCTCTACTTCTTCCTCCTGAAACTGTAGATGCTGCTCCTGCTCTTGGTCTCGGGGTC
CTCGAAGGTCTTCTTCTGGTCCGTGATGAAGTAACGACGACACCTCTCCGACACCCTGATACCGTACTCG
TCGTCGGGGGTACACGACTCCTTGTCCCGGGTCAGACCGAGACACGGGGTCAAGTTCTTCCACCACAAGG
TCCTCAAGTGACTACCGTCGAAGTGGGTCGGGGACATGTCTCCCCTCGACTTACTCGTGGACCCGGACGA
CCCGGGGATGTAGTCCCGACTCCACCTCCTGTTGTAGTACCACTGGAAGTCCTTGGTCCGGTCGTCCGGG
ATGTCGAAGATGTCGTCGGACTAGTCGATACTCCTCCTGGTCTCCGTCCCCCGACTCGGGTCCTTCTTGA
AACACTTCGGGTTACTTTGGTTCTGGATGAAGACCTTCCACGTCGTGGTGTACCGGGGGTGGTTCCTACT
CAAACTGACGTTCCGGACCCGGATGAAGAGACTACACCTGGACCTCTTCCTACACGTGAGACCGGACTAA
CCGGGGGACGACCACACGGTGTGGTTGTGGGACTTGGGACGGGTACCGTCCGTCCACTGACACGTCCTCA
AACGGGACAAGAAGTGGTAGAAACTACTTTGGTTCTCGACCATGAAGTGACTCTTGTACCTCTCCTTGAC
GTCCCGGGGGACGTTGTAGGTCTACCTCCTGGGGTGGAAGTTCCTCTTGATGTCCAAGGTACGGTAGTTA
CCGATGTAGTACCTGTGGGACGGACCGGACCACTACCGGGTCCTGGTCTCCTAGTCCACCATGGACGACT
CGTACCCGTCGTTACTCTTGTAGGTGTCGTAGGTGAAGAGACCGGTACACAAGTGACACTCCTTCTTCCT
CCTCATGTTCTACCGGGACATGTTGGACATGGGACCCCACAAACTCTGACACCTCTACGACGGGTCGTTC
CGACCGTAGACCTCCCACCTCACGGACTAACCCCTCGTGGACGTACGACCGTACTCGTGGGACAAGGACC
ACATGTCGTTGTTCACGGTCTGGGGGGACCCGTACCGGAGACCGGTGTAGTCCCTGAAGGTCTAGTGACG
GAGACCGGTCATACCGGTCACCCGGGGGTTCGACCGGTCCGACGTGATGAGACCGTCGTAGTTACGGACC
TCGTGGTTCCTCGGGAAGTCGACCTAGTTCCACCTGGACGACCGGGGGTACTAGTAGGTACCGTAGTTCT
GGGTCCCCCGGTCCGTCTTCAAGTCGTCGGACATGTAGTCGGTCAAGTAGTAGTACATGTCGGACCTACC
GTTCTTCACCGTCTGGATGTCCCCGTTGTCGTGACCGTGGGACTACCACAAGAAACCGTTACACCTGTCG
AGACCGTAGTTCGTGTTGTAGAAGTTGGGGGGGTAGTAACGGTCTATGTAGTCCGACGTGGGGTGGGTGA
TGTCGTAGTCCTCGTGGGACTCCTACCTCGACTACCCGACACTGGACTTGTCGACGTCGTACGGGGACCC
GTACCTCTCGTTCCGGTAGAGACTACGGGTCTAGTGACGGTCGTCGATGAAGTGGTTGTACAAACGGTGG
ACCTCGGGGTCGTTCCGGTCCGACGTGGACGTCCCGTCCTCGTTACGGACCTCCGGGGTCCAGTTGTTGG
GGTTCCTCACCGACGTCCACCTGAAGGTCTTCTGGTACTTCCACTGACCCCACTGGTGGGTCCCCCACTT
CTCGGACGACTGGTCGTACATACACTTCCTCAAGGACTAGTCGTCGTCGGTCCTACCGGTGGTCACCTGG
GACAAGAAGGTCTTACCGTTCCACTTCCACAAGGTCCCGTTGGTCCTGTCGAAGTGGGGACACCACTTGT
CGGACCTGGGGGGGGACGACTGGTCTATGGACTCCTAAGTGGGGGTCTCGACCCACGTGGTCTAACGGGA
CTCCTACCTCCACGACCCGACACTCCGGGTCCTGGACATGACT
SEQ ID NO: 16 Complementary strand to the exemplified FVIII transgene (V3)
TACGTCTAACTCGACTCGTGGACGAAGAAGGACACGGACGACTCCAAGACGAAGAGACGGTGGTCCTCTA
TGATGGACCCCCGACACCTCGACTCGACCCTGATGTACGTCAGACTGGACCCCCTCGACGGACACCTACG
GTCCAAGGGGGGGTCTCACGGGTTCTCGAAGGGGAAGTTGTGGAGACACCACATGTTCTTCTGGGACAAA
CACCTCAAGTGACTGGTGGACAAGTTGTAACGGTTCGGGTCCGGGGGGACCTACCCGGACGACCCGGGGT
GGTAGGTCCGACTCCACATACTGTGACACCACTAGTGGGACTTCTTGTACCGGTCGGTGGGACACTCGGA
CGTACGACACCCCCACTCGATGACCTTCCGGAGACTCCCCCGACTCATACTACTGGTCTGGTCGGTCTCC
CTCTTCCTCCTACTGTTCCACAAGGGACCCCCGTCGGTGTGGATACACACCGTCCACGACTTCCTCTTAC
CGGGGTACCGGAGACTGGGGGACACGGACTGGATGTCGATGGACTCGGTACACCTGGACCACTTCCTGGA
CTTGAGACCGGACTAACCCCGGGACGACCACACGTCCCTCCCGTCGGACCGGTTCCTCTTCTGGGTCTGG
GACGTGTTCAAGTAGGACGACAAACGACACAAACTACTCCCGTTCTCGACCGTGAGACTTTGGTTCTTGT
CGGACTACGTCCTGTCCCTACGACGGAGACGGTCCCGGACCGGGTTCTACGTGTGACACTTACCGATACA
CTTGTCCTCGGACGGACCGGACTAACCGACGGTGTCCTTCAGACACATGACCGTACACTAACCGTACCCG
TGGTGGGGACTCCACGTGTCGTAGAAGGACCTCCCGGTGTGGAAGGACCAGTCCTTGGTGTCCGTCCGGT
CGGACCTCTAGTCGGGGTAGTGGAAGGACTGACGGGTCTGGGACGACTACCTGGACCCGGTCAAGGACGA
CAAGACGGTGTAGTCGTCGGTGGTCGTACTACCGTACCTCCGGATACACTTCCACCTGTCGACGGGACTC
CTCGGGGTCGACTCCTACTTCTTGTTACTCCTCCGACTCCTGATACTACTACTGGACTGACTGAGACTCT
ACCTACACCACTCCAAACTACTACTGTTGTCGGGGTCGAAGTAGGTCTAGTCCAGACACCGGTTCTTCGT
GGGGTTCTGGACCCACGTGATGTAACGACGACTCCTCCTCCTGACCCTGATACGGGGGGACCACGACCGG
GGACTACTGTCCTCGATGTTCTCGGTCATGGACTTGTTACCGGGGGTCTCCTAACCGTCCTTCATGTTCT
TCCAGTCCAAGTACCGGATGTGACTACTTTGGAAGTTCTGGTCCCTCCGGTAGGTCGTACTCAGACCGTA
GGACCCGGGGGACGACATACCCCTCCACCCCCTGTGGGACGACTAGTAGAAGTTCTTGGTCCGGTCGTCC
GGGATGTTGTAGATGGGGGTACCGTAGTGACTACACTCCGGGGACATGTCGTCCTCCGACGGGTTCCCCC
ACTTCGTGGACTTCCTGAAGGGGTAGGACGGACCCCTCTAGAAGTTCATGTTCACCTGACACTGACACCT
CCTACCGGGGTGGTTCAGACTGGGGTCCACGGACTGGTCTATGATGTCGTCGAAACACTTGTACCTCTCC
CTGGACCGGAGACCGGACTAACCGGGGGACGACTAGACGATGTTCCTCAGACACCTGGTCTCCCCGTTGG
TCTAGTACAGACTGTTCTCCTTACACTAGGACAAGAGACACAAACTACTCTTGTCCTCGACCATGGACTG
ACTCTTGTAGGTCTCCAAGGACGGGTTGGGACGACCCCACGTCGACCTCCTGGGACTCAAGGTCCGGTCG
TTGTAGTACGTGTCGTAGTTACCGATACACAAACTGTCGGACGTCGACAGACACACGGACGTACTCCACC
GGATGACCATGTAGGACTCGTAACCCCGGGTCTGACTGAAGGACAGACACAAGAAGAGACCGATGTGGAA
GTTCGTGTTCTACCACATACTCCTGTGGGACTGGGACAAGGGGAAGAGACCCCTCTGACACAAGTACTCG
TACCTCTTGGGACCGGACACCTAAGACCCGACGGTGTTGAGACTGAAGTCCTTGTCCCCGTACTGACGGG
ACGACTTTCAGAGGTCGACACTGTTCTTGTGACCCCTGATGATACTCCTGTCGATACTCCTGTAGAGACG
GATGGACGACTCGTTCTTGTTACGGTAACTCGGGTCCTCGAAGTCGGTCTTACGGTGATTACACAGATTG
TTGTCGTTGTGGTCGTTACTGTCGTTACACAGAGGGGGTCACGACTTCTCCGTGGTCTCCCTCTAGTGGT
CCTGGTGGGACGTCAGACTGGTCCTCCTCTAACTGATACTACTGTGGTAGAGACACCTCTACTTCTTCCT
CCTGAAACTGTAGATGCTGCTCCTGCTCTTGGTCTCGGGGTCCTCGAAGGTCTTCTTCTGGTCCGTGATG
AAGTAACGACGACACCTCTCCGACACCCTGATACCGTACTCGTCGTCGGGGGTACACGACTCCTTGTCCC
GGGTCAGACCGAGACACGGGGTCAAGTTCTTCCACCACAAGGTCCTCAAGTGACTACCGTCGAAGTGGGT
CGGGGACATGTCTCCCCTCGACTTACTCGTGGACCCGGACGACCCGGGGATGTAGTCCCGACTCCACCTC
CTGTTGTAGTACCACTGGAAGTCCTTGGTCCGGTCGTCCGGGATGTCGAAGATGTCGTCGGACTAGTCGA
TACTCCTCCTGGTCTCCGTCCCCCGACTCGGGTCCTTCTTGAAACACTTCGGGTTACTTTGGTTCTGGAT
GAAGACCTTCCACGTCGTGGTGTACCGGGGGTGGTTCCTACTCAAACTGACGTTCCGGACCCGGATGAAG
AGACTACACCTGGACCTCTTCCTACACGTGAGACCGGACTAACCGGGGGACGACCACACGGTGTGGTTGT
GGGACTTGGGACGGGTACCGTCCGTCCACTGACACGTCCTCAAACGGGACAAGAAGTGGTAGAAACTACT
TTGGTTCTCGACCATGAAGTGACTCTTGTACCTCTCCTTGACGTCCCGGGGGACGTTGTAGGTCTACCTC
CTGGGGTGGAAGTTCCTCTTGATGTCCAAGGTACGGTAGTTACCGATGTAGTACCTGTGGGACGGACCGG
ACCACTACCGGGTCCTGGTCTCCTAGTCCACCATGGACGACTCGTACCCGTCGTTACTCTTGTAGGTGTC
GTAGGTGAAGAGACCGGTACACAAGTGACACTCCTTCTTCCTCCTCATGTTCTACCGGGACATGTTGGAC
ATGGGACCCCACAAACTCTGACACCTCTACGACGGGTCGTTCCGACCGTAGACCTCCCACCTCACGGACT
AACCCCTCGTGGACGTACGACCGTACTCGTGGGACAAGGACCACATGTCGTTGTTCACGGTCTGGGGGGA
CCCGTACCGGAGACCGGTGTAGTCCCTGAAGGTCTAGTGACGGAGACCGGTCATACCGGTCACCCGGGGG
TTCGACCGGTCCGACGTGATGAGACCGTCGTAGTTACGGACCTCGTGGTTCCTCGGGAAGTCGACCTAGT
TCCACCTGGACGACCGGGGGTACTAGTAGGTACCGTAGTTCTGGGTCCCCCGGTCCGTCTTCAAGTCGTC
GGACATGTAGTCGGTCAAGTAGTAGTACATGTCGGACCTACCGTTCTTCACCGTCTGGATGTCCCCGTTG
TCGTGACCGTGGGACTACCACAAGAAACCGTTACACCTGTCGAGACCGTAGTTCGTGTTGTAGAAGTTGG
GGGGGTAGTAACGGTCTATGTAGTCCGACGTGGGGTGGGTGATGTCGTAGTCCTCGTGGGACTCCTACCT
CGACTACCCGACACTGGACTTGTCGACGTCGTACGGGGACCCGTACCTCTCGTTCCGGTAGAGACTACGG
GTCTAGTGACGGTCGTCGATGAAGTGGTTGTACAAACGGTGGACCTCGGGGTCGTTCCGGTCCGACGTGG
ACGTCCCGTCCTCGTTACGGACCTCCGGGGTCCAGTTGTTGGGGTTCCTCACCGACGTCCACCTGAAGGT
CTTCTGGTACTTCCACTGACCCCACTGGTGGGTCCCCCACTTCTCGGACGACTGGTCGTACATACACTTC
CTCAAGGACTAGTCGTCGTCGGTCCTACCGGTGGTCACCTGGGACAAGAAGGTCTTACCGTTCCACTTCC
ACAAGGTCCCGTTGGTCCTGTCGAAGTGGGGACACCACTTGTCGGACCTGGGGGGGGACGACTGGTCTAT
GGACTCCTAAGTGGGGGTCTCGACCCACGTGGTCTAACGGGACTCCTACCTCCACGACCCGACACTCCGG
GTCCTGGACATGACT
SEQ ID NO: 17 Exemplified FVIII polypeptide (N6)
MQIELSTCFFLCLLRFCFSATRRYYLGAVELSWDYMQSDLGELPVDARFPPRVPKSFPFNTSVVYKKTLFV
EFTDHLFNIAKPRPPWMGLLGPTIQAEVYDTVVITLKNMASHPVSLHAVGVSYWKASEGAEYDDQTSQREK
EDDKVFPGGSHTYVWQVLKENGPMASDPLCLTYSYLSHVDLVKDLNSGLIGALLVCREGSLAKEKTQTLHK
FILLFAVEDEGKSWHSETKNSLMQDRDAASARAWPKMHTVNGYVNRSLPGLIGCHRKSVYWHVIGMGTTPE
VHSIFLEGHTFLVRNHRQASLEISPITFLTAQTLLMDLGQFLLFCHISSHQHDGMEAYVKVDSCPEEPQLR
MKNNEEAEDYDDDLTDSEMDVVRFDDDNSPSFIQIRSVAKKHPKTWVHYIAAEEEDWDYAPLVLAPDDRSY
KSQYLNNGPQRIGRKYKKVRFMAYTDETFKTREAIQHESGILGPLLYGEVGDTLLIIFKNQASRPYNIYPH
GITDVRPLYSRRLPKGVKHLKDFPILPGEIFKYKWTVTVEDGPTKSDPRCLTRYYSSFVNMERDLASGLIG
PLLICYKESVDQRGNQIMSDKRNVILFSVEDENRSWYLTENIQRFLPNPAGVQLEDPEFQASNIMHSINGY
VFDSLQLSVCLHEVAYWYILSIGAQTDFLSVFFSGYTFKHKMVYEDTLTLFPFSGETVFMSMENPGLWILG
CHNSDFRNRGMTALLKVSSCDKNTGDYYEDSYEDISAYLLSKNNAIEPRSFSQNSRHPSTRQKQFNATTIP
ENDIEKTDPWFAHRTPMPKIQNVSSSDLLMLLRQSPTPHGLSLSDLQEAKYETFSDDPSPGAIDSNNSLSE
MTHFRPQLHHSGDMVFTPESGLQLRLNEKLGTTAATELKKLDFKVSSTSNNLISTIPSDNLAAGTDNTSSL
GPPSMPVHYDSQLDTTLFGKKSSPLTESGGPLSLSEENNDSKLLESGLMNSQESSWGKNVSSREITRTTLQ
SDQEEIDYDDTISVEMKKEDEDIYDEDENQSPRSFQKKTRHYFIAAVERLWDYGMSSSPHVLRNRAQSGSV
PQFKKVVFQEFTDGSFTQPLYRGELNEHLGLLGPYIRAEVEDNIMVTFRNQASRPYSFYSSLISYEEDQRQ
GAEPRKNFVKPNETKTYFWKVQHHMAPTKDEFDCKAWAYFSDVDLEKDVHSGLIGPLLVCHTNTLNPAHGR
QVTVQEFALFFTIFDETKSWYFTENMERNCRAPCNIQMEDPTFKENYRFHAINGYIMDTLPGLVMAQDQRI
RWYLLSMGSNENIHSIHFSGHVFTVRKKEEYKMALYNLYPGVFETVEMLPSKAGIWRVECLIGEHLHAGMS
TLFLVYSNKCQTPLGMASGHIRDFQITASGQYGQWAPKLARLHYSGSINAWSTKEPFSWIKVDLLAPMIIH
GIKTQGARQKFSSLYISQFIIMYSLDGKKWQTYRGNSTGTLMVFFGNVDSSGIKHNIENPPIIARYIRLHP
THYSIRSTLRMELMGCDLNSCSMPLGMESKAISDAQITASSYFTNMFATWSPSKARLHLQGRSNAWRPQVN
NPKEWLQVDFQKTMKVTGVTTQGVKSLLTSMYVKEFLISSSQDGHQWTLFFQNGKVKVFQGNQDSFTPVVN
SLDPPLLTRYLRIHPQSWVHQIALRMEVLGCEAQDLY
Signal peptide is underlined.
SEQ ID NO: 18 Exemplified FVIII polypeptide (V3)
MQIELSTCFFLCLLRFCFSATRRYYLGAVELSWDYMQSDLGELPVDARFPPRVPKSFPENTSVVYKKTLF
VEFTDHLFNIAKPRPPWMGLLGPTIQAEVYDTVVITLKNMASHPVSLHAVGVSYWKASEGAEYDDQTSQR
EKEDDKVFPGGSHTYVWQVLKENGPMASDPLCLTYSYLSHVDLVKDLNSGLIGALLVCREGSLAKEKTQT
LHKFILLFAVFDEGKSWHSETKNSLMQDRDAASARAWPKMHTVNGYVNRSLPGLIGCHRKSVYWHVIGMG
TTPEVHSIFLEGHTFLVRNHRQASLEISPITFLTAQTLLMDLGQFLLFCHISSHQHDGMEAYVKVDSCPE
EPQLRMKNNEEAEDYDDDLTDSEMDVVRFDDDNSPSFIQIRSVAKKHPKTWVHYIAAEEEDWDYAPLVLA
PDDRSYKSQYLNNGPQRIGRKYKKVRFMAYTDETFKTREAIQHESGILGPLLYGEVGDTLLIIFKNQASR
PYNIYPHGITDVRPLYSRRLPKGVKHLKDFPILPGEIFKYKWTVTVEDGPTKSDPRCLTRYYSSFVNMER
DLASGLIGPLLICYKESVDQRGNQIMSDKRNVILFSVFDENRSWYLTENIQRFLPNPAGVQLEDPEFQAS
NIMHSINGYVFDSLQLSVCLHEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLLSKNNAIEPRSFSQNATNVSN
NSNTSNDSNVSPPVLKRHQREITRTTLQSDQEEIDYDDTISVEMKKEDFDIYDEDENQSPRSFQKKTRHY
FIAAVERLWDYGMSSSPHVLRNRAQSGSVPQFKKVVFQEFTDGSFTQPLYRGELNEHLGLLGPYIRAEVE
DNIMVTFRNQASRPYSFYSSLISYEEDQRQGAEPRKNFVKPNETKTYFWKVQHHMAPTKDEFDCKAWAYF
SDVDLEKDVHSGLIGPLLVCHTNTLNPAHGRQVTVQEFALFFTIFDETKSWYFTENMERNCRAPCNIQME
DPTFKENYRFHAINGYIMDTLPGLVMAQDQRIRWYLLSMGSNENIHSIHFSGHVFTVRKKEEYKMALYNL
YPGVFETVEMLPSKAGIWRVECLIGEHLHAGMSTLFLVYSNKCQTPLGMASGHIRDFQITASGQYGQWAP
KLARLHYSGSINAWSTKEPFSWIKVDLLAPMIIHGIKTQGARQKFSSLYISQFIIMYSLDGKKWQTYRGN
STGTLMVFFGNVDSSGIKHNIFNPPIIARYIRLHPTHYSIRSTLRMELMGCDLNSCSMPLGMESKAISDA
QITASSYFTNMFATWSPSKARLHLQGRSNAWRPQVNNPKEWLQVDFQKTMKVTGVTTQGVKSLLTSMYVK
EFLISSSQDGHQWTLFFQNGKVKVFQGNQDSFTPVVNSLDPPLLTRYLRIHPQSWVHQIALRMEVLGCEA
QDLY
Signal peptide is underlined.
SEQ ID NO: 19 Exemplified Human DCN (Decorin) transgene
ATGAAGGCCACTATCATCCTCCTTCTGCTTGCACAAGTTTCCTGGGCTGGACCGTTTCAACAGAGAGGCTTATTT
GACTTTATGCTAGAAGATGAGGCTTCTGGGATAGGCCCAGAAGTTCCTGATGACCGCGACTTCGAGCCCTCCCTA
GGCCCAGTGTGCCCCTTCCGCTGTCAATGCCATCTTCGAGTGGTCCAGTGTTCTGATTTGGGTCTGGACAAAGTG
CCAAAGGATCTTCCCCCTGACACAACTCTGCTAGACCTGCAAAACAACAAAATAACCGAAATCAAAGATGGAGAC
TTTAAGAACCTGAAGAACCTTCACGCATTGATTCTTGTCAACAATAAAATTAGCAAAGTTAGTCCTGGAGCATTT
ACACCTTTGGTGAAGTTGGAACGACTTTATCTGTCCAAGAATCAGCTGAAGGAATTGCCAGAAAAAATGCCCAAA
ACTCTTCAGGAGCTGCGTGCCCATGAGAATGAGATCACCAAAGTGCGAAAAGTTACTTTCAATGGACTGAACCAG
ATGATTGTCATAGAACTGGGCACCAATCCGCTGAAGAGCTCAGGAATTGAAAATGGGGCTTTCCAGGGAATGAAG
AAGCTCTCCTACATCCGCATTGCTGATACCAATATCACCAGCATTCCTCAAGGTCTTCCTCCTTCCCTTACGGAA
TTACATCTTGATGGCAACAAAATCAGCAGAGTTGATGCAGCTAGCCTGAAAGGACTGAATAATTTGGCTAAGTTG
GGATTGAGTTTCAACAGCATCTCTGCTGTTGACAATGGCTCTCTGGCCAACACGCCTCATCTGAGGGAGCTTCAC
TTGGACAACAACAAGCTTACCAGAGTACCTGGTGGGCTGGCAGAGCATAAGTACATCCAGGTTGTCTACCTTCAT
AACAACAATATCTCTGTAGTTGGATCAAGTGACTTCTGCCCACCTGGACACAACACCAAAAAGGCTTCTTATTCG
GGTGTGAGTCTTTTCAGCAACCCGGTCCAGTACTGGGAGATACAGCCATCCACCTTCAGATGTGTCTACGTGCGC
TCTGCCATTCAACTCGGAAACTATAAGTAA
Nucleic acid sequence encoding signal peptide is underlined.
SEQ ID NO: 20 Exemplified Human Decorin polypeptide
MKATIILLLLAQVSWAGPFQQRGLFDFMLEDEASGIGPEVPDDRDFEPSLGPVCPFRCQCHLRVVQCSDLGLDKV
PKDLPPDTTLLDLQNNKITEIKDGDFKNLKNLHALILVNNKISKVSPGAFTPLVKLERLYLSKNQLKELPEKMPK
TLQELRAHENEITKVRKVTFNGLNQMIVIELGTNPLKSSGIENGAFQGMKKLSYIRIADTNITSIPQGLPPSLTE
LHLDGNKISRVDAASLKGLNNLAKLGLSENSISAVDNGSLANTPHLRELHLDNNKLTRVPGGLAEHKYIQVVYLH
NNNISVVGSSDFCPPGHNTKKASYSGVSLFSNPVQYWEIQPSTFRCVYVRSAIQLGNYK
Nucleic acid sequence encoding signal peptide is underlined.
SEQ ID NO: 21 Exemplified Human TRIM72 transgene
ATGTCGGCTGCGCCCGGCCTCCTGCACCAGGAGCTGTCCTGCCCGCTGTGCCTGCAGCTGTTCGACGCGCCCGTG
ACAGCCGAGTGCGGCCACAGTTTCTGCCGCGCCTGCCTAGGCCGCGTGGCCGGGGAGCCGGCGGCGGATGGCACC
GTTCTCTGCCCCTGCTGCCAGGCCCCCACGCGGCCGCAGGCACTCAGCACCAACCTGCAGCTGGCGCGCCTGGTG
GAGGGGCTGGCCCAGGTGCCGCAGGGCCACTGCGAGGAGCACCTGGACCCGCTGAGCATCTACTGCGAGCAGGAC
CGCGCGCTGGTGTGCGGAGTGTGCGCCTCACTCGGCTCGCACCGCGGTCATCGCCTCCTGCCTGCCGCCGAGGCC
CACGCACGCCTCAAGACACAGCTGCCACAGCAGAAACTGCAGCTGCAGGAGGCATGCATGCGCAAGGAGAAGAGT
GTGGCTGTGCTGGAGCATCAGCTGGTGGAGGTGGAGGAGACAGTGCGTCAGTTCCGGGGGGCCGTGGGGGAGCAG
CTGGGCAAGATGCGGGTGTTCCTGGCTGCACTGGAGGGCTCCTTGGACCGCGAGGCAGAGCGTGTACGGGGTGAG
GCAGGGGTCGCCTTGCGCCGGGAGCTGGGGAGCCTGAACTCTTACCTGGAGCAGCTGCGGCAGATGGAGAAGGTC
CTGGAGGAGGTGGCGGACAAGCCGCAGACTGAGTTCCTCATGAAATACTGCCTGGTGACCAGCAGGCTGCAGAAG
ATCCTGGCAGAGTCTCCCCCACCCGCCCGTCTGGACATCCAGCTGCCAATTATCTCAGATGACTTCAAATTCCAG
GTGTGGAGGAAGATGTTCCGGGCTCTGATGCCAGCGCTGGAGGAGCTGACCTTTGACCCGAGCTCTGCGCACCCG
AGCCTGGTGGTGTCTTCCTCTGGCCGCCGCGTGGAGTGCTCGGAGCAGAAGGCGCCGCCGGCCGGGGAGGACCCG
CGCCAGTTCGACAAGGCGGTGGCGGTGGTGGCGCACCAGCAGCTCTCCGAGGGCGAGCACTACTGGGAGGTGGAT
GTTGGCGACAAGCCGCGCTGGGCGCTGGGCGTGATCGCGGCCGAGGCCCCCCGCCGCGGGCGCCTGCACGCGGTG
CCCTCGCAGGGCCTGTGGCTGCTGGGGCTGCGCGAGGGCAAGATCCTGGAGGCACACGTGGAGGCCAAGGAGCCG
CGCGCTCTGCGCAGCCCCGAGAGGCGGCCCACGCGCATTGGCCTTTACCTGAGCTTCGGCGACGGCGTCCTCTCC
TTCTACGATGCCAGCGACGCCGACGCGCTCGTGCCGCTTTTTGCCTTCCACGAGCGCCTGCCCAGGCCCGTGTAC
CCCTTCTTCGACGTGTGCTGGCACGACAAGGGCAAGAATGCCCAGCCGCTGCTGCTCGTGGGTCCCGAAGGCGCC
GAGGCCTGA
SEQ ID NO: 22 Exemplified Human TRIM72 polypeptide
MSAAPGLLHQELSCPLCLQLFDAPVTAECGHSFCRACLGRVAGEPAADGTVLCPCCQAPTRPQALSTNLQLARLV
EGLAQVPQGHCEEHLDPLSIYCEQDRALVCGVCASLGSHRGHRLLPAAEAHARLKTQLPQQKLQLQEACMRKEKS
VAVLEHQLVEVEETVRQFRGAVGEQLGKMRVFLAALEGSLDREAERVRGEAGVALRRELGSLNSYLEQLRQMEKV
LEEVADKPQTEFLMKYCLVTSRLQKILAESPPPARLDIQLPIISDDFKFQVWRKMFRALMPALEELTFDPSSAHP
SLVVSSSGRRVECSEQKAPPAGEDPRQFDKAVAVVAHQQLSEGEHYWEVDVGDKPRWALGVIAAEAPRRGRLHAV
PSQGLWLLGLREGKILEAHVEAKEPRALRSPERRPTRIGLYLSFGDGVLSFYDASDADALVPLFAFHERLPRPVY
PFFDVCWHDKGKNAQPLLLVGPEGAEA
SEQ ID NO: 23 Exemplified Human ABACA3 (ABCA3) transgene
ATGGCTGTGCTCAGGCAGCTGGCGCTCCTCCTCTGGAAGAACTACACCCTGCAGAAGCGGAAGGTCCTGGTGACG
GTCCTGGAACTCTTCCTGCCATTGCTGTTTTCTGGGATCCTCATCTGGCTCCGCTTGAAGATTCAGTCGGAAAAT
GTGCCCAACGCCACCATCTACCCGGGCCAGTCCATCCAGGAGCTGCCTCTGTTCTTCACCTTCCCTCCGCCAGGA
GACACCTGGGAGCTTGCCTACATCCCTTCTCACAGTGACGCTGCCAAGACCGTCACTGAGACAGTGCGCAGGGCA
CTTGTGATCAACATGCGAGTGCGCGGCTTTCCCTCCGAGAAGGACTTTGAGGACTACATTAGGTACGACAACTGC
TCGTCCAGCGTGCTGGCCGCCGTGGTCTTCGAGCACCCCTTCAACCACAGCAAGGAGCCCCTGCCGCTGGCGGTG
AAATATCACCTACGGTTCAGTTACACACGGAGAAATTACATGTGGACCCAAACAGGCTCCTTTTTCCTGAAAGAG
ACAGAAGGCTGGCACACTACTTCCCTTTTCCCGCTTTTCCCAAACCCAGGACCAAGGGAACCTACATCCCCTGAT
GGCGGAGAACCTGGGTACATCCGGGAAGGCTTCCTGGCCGTGCAGCATGCTGTGGACCGGGCCATCATGGAGTAC
CATGCCGATGCCGCCACACGCCAGCTGTTCCAGAGACTGACGGTGACCATCAAGAGGTTCCCGTACCCGCCGTTC
ATCGCAGACCCCTTCCTCGTGGCCATCCAGTACCAGCTGCCCCTGCTGCTGCTGCTCAGCTTCACCTACACCGCG
CTCACCATTGCCCGTGCTGTCGTGCAGGAGAAGGAAAGGAGGCTGAAGGAGTACATGCGCATGATGGGGCTCAGC
AGCTGGCTGCACTGGAGTGCCTGGTTCCTCTTGTTCTTCCTCTTCCTCCTCATCGCCGCCTCCTTCATGACCCTG
CTCTTCTGTGTCAAGGTGAAGCCAAATGTAGCCGTGCTGTCCCGCAGCGACCCCTCCCTGGTGCTCGCCTTCCTG
CTGTGCTTCGCCATCTCTACCATCTCCTTCAGCTTCATGGTCAGCACCTTCTTCAGCAAAGCCAACATGGCAGCA
GCCTTCGGAGGCTTCCTCTACTTCTTCACCTACATCCCCTACTTCTTCGTGGCCCCTCGGTACAACTGGATGACT
CTGAGCCAGAAGCTCTGCTCCTGCCTCCTGTCTAATGTCGCCATGGCAATGGGAGCCCAGCTCATTGGGAAATTT
GAGGCGAAAGGCATGGGCATCCAGTGGCGAGACCTCCTGAGTCCCGTCAACGTGGACGACGACTTCTGCTTCGGG
CAGGTGCTGGGGATGCTGCTGCTGGACTCTGTGCTCTATGGCCTGGTGACCTGGTACATGGAGGCCGTCTTCCCA
GGGCAGTTCGGCGTGCCTCAGCCCTGGTACTTCTTCATCATGCCCTCCTATTGGTGTGGGAAGCCAAGGGCGGTT
GCAGGGAAGGAGGAAGAAGACAGTGACCCCGAGAAAGCACTCAGAAACGAGTACTTTGAAGCCGAGCCAGAGGAC
CTGGTGGCGGGGATCAAGATCAAGCACCTGTCCAAGGTGTTCAGGGTGGGAAATAAGGACAGGGCGGCCGTCAGA
GACCTGAACCTCAACCTGTACGAGGGACAGATCACCGTCCTGCTGGGCCACAACGGTGCCGGGAAGACCACCACC
CTCTCCATGCTCACAGGTCTCTTTCCCCCCACCAGTGGACGGGCATACATCAGCGGGTATGAAATTTCCCAGGAC
ATGGTTCAGATCCGGAAGAGCCTGGGCCTGTGCCCGCAGCACGACATCCTGTTTGACAACTTGACAGTCGCAGAG
CACCTTTATTTCTACGCCCAGCTGAAGGGCCTGTCACGTCAGAAGTGCCCTGAAGAAGTCAAGCAGATGCTGCAC
ATCATCGGCCTGGAGGACAAGTGGAACTCACGGAGCCGCTTCCTGAGCGGGGGCATGAGGCGCAAGCTCTCCATC
GGCATCGCCCTCATCGCAGGCTCCAAGGTGCTGATACTGGACGAGCCCACCTCGGGCATGGACGCCATCTCCAGG
AGGGCCATCTGGGATCTTCTTCAGCGGCAGAAAAGTGACCGCACCATCGTGCTGACCACCCACTTCATGGACGAG
GCTGACCTGCTGGGAGACCGCATCGCCATCATGGCCAAGGGGGAGCTGCAGTGCTGCGGGTCCTCGCTGTTCCTC
AAGCAGAAATACGGTGCCGGCTATCACATGACGCTGGTGAAGGAGCCGCACTGCAACCCGGAAGACATCTCCCAG
CTGGTCCACCACCACGTGCCCAACGCCACGCTGGAGAGCAGCGCTGGGGCCGAGCTGTCTTTCATCCTTCCCAGA
GAGAGCACGCACAGGTTTGAAGGTCTCTTTGCTAAACTGGAGAAGAAGCAGAAAGAGCTGGGCATTGCCAGCTTT
GGGGCATCCATCACCACCATGGAGGAAGTCTTCCTTCGGGTCGGGAAGCTGGTGGACAGCAGTATGGACATCCAG
GCCATCCAGCTCCCTGCCCTGCAGTACCAGCACGAGAGGCGCGCCAGCGACTGGGCTGTGGACAGCAACCTCTGT
GGGGCCATGGACCCCTCCGACGGCATTGGAGCCCTCATCGAGGAGGAGCGCACCGCTGTCAAGCTCAACACTGGG
CTCGCCCTGCACTGCCAGCAATTCTGGGCCATGTTCCTGAAGAAGGCCGCATACAGCTGGCGCGAGTGGAAAATG
GTGGCGGCACAGGTCCTGGTGCCTCTGACCTGCGTCACCCTGGCCCTCCTGGCCATCAACTACTCCTCGGAGCTC
TTCGACGACCCCATGCTGAGGCTGACCTTGGGCGAGTACGGCAGAACCGTCGTGCCCTTCTCAGTTCCCGGGACC
TCCCAGCTGGGTCAGCAGCTGTCAGAGCATCTGAAAGACGCACTGCAGGCTGAGGGACAGGAGCCCCGCGAGGTG
CTCGGTGACCTGGAGGAGTTCTTGATCTTCAGGGCTTCTGTGGAGGGGGGCGGCTTTAATGAGCGGTGCCTTGTG
GCAGCGTCCTTCAGAGATGTGGGAGAGCGCACGGTCGTCAACGCCTTGTTCAACAACCAGGCGTACCACTCTCCA
GCCACTGCCCTGGCCGTCGTGGACAACCTTCTGTTCAAGCTGCTGTGCGGGCCTCACGCCTCCATTGTGGTCTCC
AACTTCCCCCAGCCCCGGAGCGCCCTGCAGGCTGCCAAGGACCAGTTTAACGAGGGCCGGAAGGGATTCGACATT
GCCCTCAACCTGCTCTTCGCCATGGCATTCTTGGCCAGCACGTTCTCCATCCTGGCGGTCAGCGAGAGGGCCGTG
CAGGCCAAGCATGTGCAGTTTGTGAGTGGAGTCCACGTGGCCAGTTTCTGGCTCTCTGCTCTGCTGTGGGACCTC
ATCTCCTTCCTCATCCCCAGTCTGCTGCTGCTGGTGGTGTTTAAGGCCTTCGACGTGCGTGCCTTCACGCGGGAC
GGCCACATGGCTGACACCCTGCTGCTGCTCCTGCTCTACGGCTGGGCCATCATCCCCCTCATGTACCTGATGAAC
TTCTTCTTCTTGGGGGCGGCCACTGCCTACACGAGGCTGACCATCTTCAACATCCTGTCAGGCATCGCCACCTTC
CTGATGGTCACCATCATGCGCATCCCAGCTGTAAAACTGGAAGAACTTTCCAAAACCCTGGATCACGTGTTCCTG
GTGCTGCCCAACCACTGTCTGGGGATGGCAGTCAGCAGTTTCTACGAGAACTACGAGACGCGGAGGTACTGCACC
TCCTCCGAGGTCGCCGCCCACTACTGCAAGAAATATAACATCCAGTACCAGGAGAACTTCTATGCCTGGAGCGCC
CCGGGGGTCGGCCGGTTTGTGGCCTCCATGGCCGCCTCAGGGTGCGCCTACCTCATCCTGCTCTTCCTCATCGAG
ACCAACCTGCTTCAGAGACTCAGGGGCATCCTCTGCGCCCTCCGGAGGAGGCGGACACTGACAGAATTATACACC
CGGATGCCTGTGCTTCCTGAGGACCAAGATGTAGCGGACGAGAGGACCCGCATCCTGGCCCCCAGTCCGGACTCC
CTGCTCCACACACCTCTGATTATCAAGGAGCTCTCCAAGGTGTACGAGCAGCGGGTGCCCCTCCTGGCCGTGGAC
AGGCTCTCCCTCGCGGTGCAGAAAGGGGAGTGCTTCGGCCTGCTGGGCTTCAATGGAGCCGGGAAGACCACGACT
TTCAAAATGCTGACCGGGGAGGAGAGCCTCACTTCTGGGGATGCCTTTGTCGGGGGTCACAGAATCAGCTCTGAT
GTCGGAAAGGTGCGGCAGCGGATCGGCTACTGCCCGCAGTTTGATGCCTTGCTGGACCACATGACAGGCCGGGAG
ATGCTGGTCATGTACGCTCGGCTCCGGGGCATCCCTGAGCGCCACATCGGGGCCTGCGTGGAGAACACTCTGCGG
GGCCTGCTGCTGGAGCCACATGCCAACAAGCTGGTCAGGACGTACAGTGGTGGTAACAAGCGGAAGCTGAGCACC
GGCATCGCCCTGATCGGAGAGCCTGCTGTCATCTTCCTGGACGAGCCGTCCACTGGCATGGACCCCGTGGCCCGG
CGCCTGCTTTGGGACACCGTGGCACGAGCCCGAGAGTCTGGCAAGGCCATCATCATCACCTCCCACAGCATGGAG
GAGTGTGAGGCCCTGTGCACCCGGCTGGCCATCATGGTGCAGGGGCAGTTCAAGTGCCTGGGCAGCCCCCAGCAC
CTCAAGAGCAAGTTCGGCAGCGGCTACTCCCTGCGGGCCAAGGTGCAGAGTGAAGGGCAACAGGAGGCGCTGGAG
GAGTTCAAGGCCTTCGTGGACCTGACCTTTCCAGGCAGCGTCCTGGAAGATGAGCACCAAGGCATGGTCCATTAC
CACCTGCCGGGCCGTGACCTCAGCTGGGCGAAGGTTTTCGGTATTCTGGAGAAAGCCAAGGAAAAGTACGGCGTG
GACGACTACTCCGTGAGCCAGATCTCGCTGGAACAGGTCTTCCTGAGCTTCGCCCACCTGCAGCCGCCCACCGCA
GAGGAGGGGCGATGA
SEQ ID NO: 24 Exemplified Human ABCA3 polypeptide
MAVLRQLALLLWKNYTLQKRKVLVTVLELFLPLLESGILIWLRLKIQSENVPNATIYPGQSIQELPLFFTFPPPG
DTWELAYIPSHSDAAKTVTETVRRALVINMRVRGFPSEKDFEDYIRYDNCSSSVLAAVVFEHPENHSKEPLPLAV
KYHLRFSYTRRNYMWTQTGSFFLKETEGWHTTSLFPLFPNPGPREPTSPDGGEPGYIREGFLAVQHAVDRAIMEY
HADAATRQLFQRLTVTIKRFPYPPFIADPFLVAIQYQLPLLLLLSFTYTALTIARAVVQEKERRLKEYMRMMGLS
SWLHWSAWFLLFFLFLLIAASFMTLLFCVKVKPNVAVLSRSDPSLVLAFLLCFAISTISFSFMVSTFFSKANMAA
AFGGFLYFFTYIPYFFVAPRYNWMTLSQKLCSCLLSNVAMAMGAQLIGKFEAKGMGIQWRDLLSPVNVDDDFCFG
QVLGMLLLDSVLYGLVTWYMEAVFPGQFGVPQPWYFFIMPSYWCGKPRAVAGKEEEDSDPEKALRNEYFEAEPED
LVAGIKIKHLSKVFRVGNKDRAAVRDLNLNLYEGQITVLLGHNGAGKTTTLSMLTGLFPPTSGRAYISGYEISQD
MVQIRKSLGLCPQHDILFDNLTVAEHLYFYAQLKGLSRQKCPEEVKQMLHIIGLEDKWNSRSRFLSGGMRRKLSI
GIALIAGSKVLILDEPTSGMDAISRRAIWDLLQRQKSDRTIVLTTHEMDEADLLGDRIAIMAKGELQCCGSSLFL
KQKYGAGYHMTLVKEPHCNPEDISQLVHHHVPNATLESSAGAELSFILPRESTHRFEGLFAKLEKKQKELGIASF
GASITTMEEVFLRVGKLVDSSMDIQAIQLPALQYQHERRASDWAVDSNLCGAMDPSDGIGALIEEERTAVKLNTG
LALHCQQFWAMFLKKAAYSWREWKMVAAQVLVPLTCVTLALLAINYSSELFDDPMLRLTLGEYGRTVVPFSVPGT
SQLGQQLSEHLKDALQAEGQEPREVLGDLEEFLIFRASVEGGGENERCLVAASFRDVGERTVVNALENNQAYHSP
ATALAVVDNLLFKLLCGPHASIVVSNFPQPRSALQAAKDQFNEGRKGFDIALNLLFAMAFLASTFSILAVSERAV
QAKHVQFVSGVHVASFWLSALLWDLISFLIPSLLLLVVFKAFDVRAFTRDGHMADTLLLLLLYGWAIIPLMYLMN
FFFLGAATAYTRLTIFNILSGIATFLMVTIMRIPAVKLEELSKTLDHVFLVLPNHCLGMAVSSFYENYETRRYCT
SSEVAAHYCKKYNIQYQENFYAWSAPGVGRFVASMAASGCAYLILLFLIETNLLQRLRGILCALRRRRTLTELYT
RMPVLPEDQDVADERTRILAPSPDSLLHTPLIIKELSKVYEQRVPLLAVDRLSLAVQKGECFGLLGENGAGKTTT
FKMLTGEESLTSGDAFVGGHRISSDVGKVRQRIGYCPQFDALLDHMTGREMLVMYARLRGIPERHIGACVENTLR
GLLLEPHANKLVRTYSGGNKRKLSTGIALIGEPAVIFLDEPSTGMDPVARRLLWDTVARARESGKAIIITSHSME
ECEALCTRLAIMVQGQFKCLGSPQHLKSKFGSGYSLRAKVQSEGQQEALEEFKAFVDLTFPGSVLEDEHQGMVHY
HLPGRDLSWAKVFGILEKAKEKYGVDDYSVSQISLEQVELSFAHLQPPTAEEGR
SEQ ID NO: 25 Exemplified WPRE component (mWPRE)

| | |
|---|---|
| 1 | GGGCCCAATC AACCTCTGGA TTACAAAATT TGTGAAAGAT TGACTGGTAT TCTTAACTAT |
| 61 | GTTGCTCCTT TTACGCTATG TGGATACGCT GCTTTAATGC CTTTGTATCA TGCTATTGCT |
| 121 | TCCCGTATGG CTTTCATTTT CTCCTCCTTG TATAAATCCT GGTTGCTGTC TCTTTATGAG |
| 181 | GAGTTGTGGC CCGTTGTCAG GCAACGTGGC GTGGTGTGCA CTGTGTTTGC TGACGCAACC |
| 241 | CCCACTGGTT GGGGCATTGC CACCACCTGT CAGCTCCTTT CCGGGACTTT CGCTTTCCCC |
| 301 | CTCCCTATTG CCACGGCGGA ACTCATCGCC GCCTGCCTTG CCCGCTGCTG GACAGGGGCT |
| 361 | CGGCTGTTGG GCACTGACAA TTCCGTGGTG TTGTCGGGGA AATCATCGTC CTTTCCTTGG |
| 421 | CTGCTCGCCT GTGTTGCCAC CTGGATTCTG CGCGGGACGT CCTTCTGCTA CGTCCCTTCG |
| 481 | GCCCTCAATC CAGCGGACCT TCCTTCCCGC GGCCTGCTGC CGGCTCTGCG GCCTCTTCCG |
| 541 | CGTCTTCGCC TTCGCCCTCA GACGAGTCGG ATCTCCCTTT GGGCCGCCTC CCCGCAAGCT |

SEQ ID NO: 26 Exemplified mifepristone-regulated promoter sequence
ACCGAGCTCTTACGCGGGTCGAAGCGGAGTACTGTCCTCCGAGTGGAGTACTGTCCTCCGAGCGGAGTACTGTCC
TCCGAGTCGAGGGTCGAAGCGGAGTACTGTCCTCCGAGTGGAGTACTGTCCTCCGAGCGGAGTACTGTCCTCCGA
GTCGACTCTAGAGGGTATATAATGGATCTCGAGATATCGGAGCTCGTTTAGTGAACCGTCAGATCGCCTGGAGAC
GCCATCCACGCTGTTTTGACCTCCATAGAAGACACCGGGACCGATCCAGCCTCCGCGGCCGGGAACGGTGCATTG
GAACGCGCATTCCCCGTGTTAATTAACAGGTAAGTGTCTTCCTCCTGTTTCCTTCCCCTGCTATTCTGCTCAACC
TTCCTATCAGAAACTGCAGTATCTGTATTTTTGCTAGCAGTAATACTAACGGTTCTTTTTTTCTCTTCACAGGCC
ACCAAGCTTGGTACCGAGCTCGGATCCACTAGTCCAGTGTGGTGGAATTCTGCAGATCGAAACGATGATAGATCC
C
SEQ ID NO: 27 Exemplified nucleic acid sequence encoding a trans-activator for
use with a mifepristone-regulated promoter
ATGGACTCCCAGCAGCCAGATCTGAAGCTACTGTCTTCTATCGAACAAGCATGCGATATTTGCCGACTTAAAAAG
CTCAAGTGCTCCAAAGAAAAACCGAAGTGCGCCAAGTGTCTGAAGAACAACTGGGAGTGTCGCTACTCTCCCAAA
ACCAAAAGGTCTCCGCTGACTAGGGCACATCTGACAGAAGTGGAATCAAGGCTAGAAAGACTGGAACAGCTATTT
CTACTGATTTTTCCTCGAGAAGACCTTGACATGATTTTGAAAATGGATTCTTTACAGGATATAAAAGCATTGTTA
GAATTCCCGGGTGTCGACCAGAAAAAGTTCAATAAAGTCAGAGTTGTGAGAGCACTGGATGCTGTTGCTCTCCCA
CAGCCAGTGGGCGTTCCAAATGAAAGCCAAGCCCTAAGCCAGAGATTCACTTTTTCACCAGGTCAAGACATACAG
TTGATTCCACCACTGATCAACCTGTTAATGAGCATTGAACCAGATGTGATCTATGCAGGACATGACAACACAAAA
CCTGACACCTCCAGTTCTTTGCTGACAAGTCTTAATCAACTAGGCGAGAGGCAACTTCTTTCAGTAGTCAAGTGG
TCTAAATCATTGCCAGGTTTTCGAAACTTACATATTGATGACCAGATAACTCTCATTCAGTATTCTTGGATGAGC
TTAATGGTGTTTGGTCTAGGATGGAGATCCTACAAACACGTCAGTGGGCAGATGCTGTATTTTGCACCTGATCTA
ATACTAAATGAACAGCGGATGAAAGAATCATCATTCTATTCATTATGCCTTACCATGTGGCAGATCCCACAGGAG
TTTGTCAAGCTTCAAGTTAGCCAAGAAGAGTTCCTCTGTATGAAAGTATTGTTACTTCTTAATACAATTCCTTTG
GAAGGGCTACGAAGTCAAACCCAGTTTGAGGAGATGAGGTCAAGCTACATTAGAGAGCTCATCAAGGCAATTGGT
TTGAGGCAAAAAGGAGTTGTGTCGAGCTCACAGCGTTTCTATCAACTTACAAAACTTCTTGATAACTTGCATGAT
CTTGTCAAACAACTTCATCTGTACTGCTTGAATACATTTATCCAGTCCCGGGCACTGAGTGTTGAATTTCCAGAA
ATGATGTCTGAAGTTATTGCTGGGTCGACGCCCATGGAATTCCAGTACCTGCCAGATACAGACGATCGTCACCGG
ATTGAGGAGAAACGTAAAAGGACATATGAGACCTTCAAGAGCATCATGAAGAAGAGTCCTTTCAGCGGACCCACC
GACCCCCGGCCTCCACCTCGACGCATTGCTGTGCCTTCCCGCAGCTCAGCTTCTGTCCCCAAGCCAGCACCCCAG
CCCTATCCCTTTACGTCATCCCTGAGCACCATCAACTATGATGAGTTTCCCACCATGGTGTTTCCTTCTGGGCAG
ATCAGCCAGGCCTCGGCCTTGGCCCCGGCCCCTCCCCAAGTCCTGCCCCAGGCTCCAGCCCCTGCCCCTGCTCCA
GCCATGGTATCAGCTCTGGCCCAGGCCCCAGCCCCTGTCCCAGTCCTAGCCCCAGGCCCTCCTCAGGCTGTGGCC
CCACCTGCCCCCAAGCCCACCCAGGCTGGGGAAGGAACGCTGTCAGAGGCCCTGCTGCAGCTGCAGTTTGATGAT
GAAGACCTGGGGGCCTTGCTTGGCAACAGCACAGACCCAGCTGTGTTCACAGACCTGGCATCCGTCGACAACTCC
GAGTTTCAGCAGCTGCTGAACCAGGGCATACCTGTGGCCCCCCACACAACTGAGCCCATGCTGATGGAGTACCCT
GAGGCTATAACTCGCCTAGTGACAGGGGCCCAGAGGCCCCCCGACCCAGCTCCTGCTCCACTGGGGGCCCCGGGG
CTCCCCAATGGCCTCCTTTCAGGAGATGAAGACTTCTCCTCCATTGCGGACATGGACTTCTCAGCCCTGCTGAGT
CAGATCAGCTCCTAA

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1-Bone Marrow Derived Macrophages (BMDM) Express Transgenes when Transduced with Lentivirus

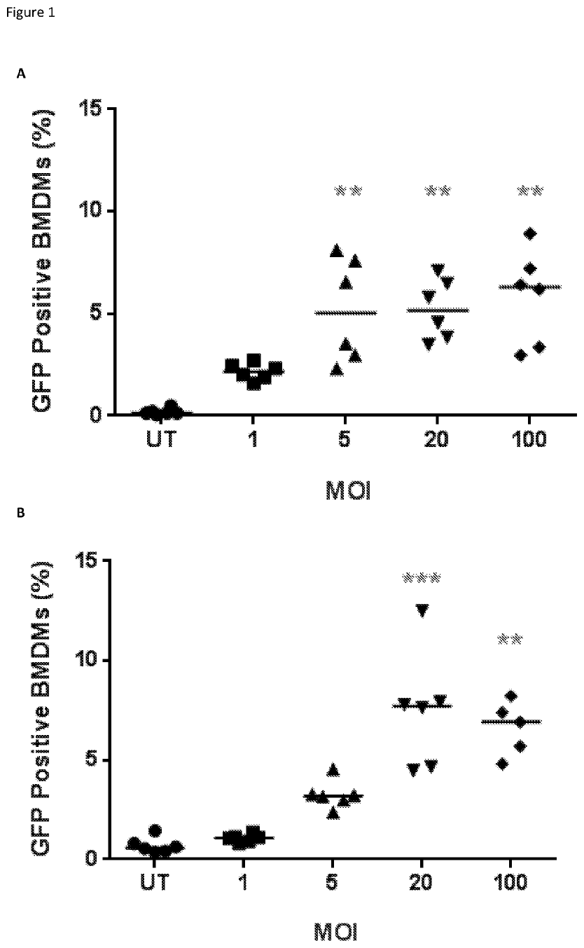

BMDM from C57BL/6 mice were transduced with F/HN pseudotyped simian immunodeficiency virus (SIV) expressing an EGFP transgene (under the control of an hCEF promoter) at increasing multiplicities of infection (MOI, 1, 5, 20, and 100). Bone marrow was differentiated into macrophages by supplying M-CSF (100 ng/ml) in culture. Cells were either a) transduced process by adding the virus with the M-CSF for 7 days (i.e. during the process of differentiating bone marrow into macrophages), or b) transduced on day 7 (i.e. after cells were differentiated), n=6/MOI. Cells were harvested 7 days post viral transduction and the percentage of EGFP positive macrophages was determined by flow cytometry (BD Fortessa) using an extended antibody panel (CD45⁺, CD11b⁺, F4/80⁺, CD64⁺, and CD24⁻) to identify bone marrow derived macrophages. The results are shown in FIG. 1A (during differentiation) and 1B (post-differentiation).

The experiment was repeated using SIV-F/HN expressing a GM-CSF transgene, again added during or post-differentiation, as for EGFP. On day 16, GM-CSF levels were measured in 2-day old media using a mouse GM-CSF Quantikine ELISA kit (R&D systems). Media volume was controlled across samples.

The results showed greater transduction efficiency of cells post-differentiation which occurred in a dose-related manner for both GFP (FIG. 1) and GM-CSF (FIG. 2) transgenes. When comparing the number of GFP positive cells as well as GM-CSF protein levels secreted into cell media (for post-differentiation transduction), the observed dose response peaked at an MOI of 20.

### Example 2—F/HN Lentivirus has a Maximum Feasible Dose of MOI 50 in BMDM

The method of Example 1 was repeated transducing only macrophages post-differentiation to confirm the maximum feasible dose.

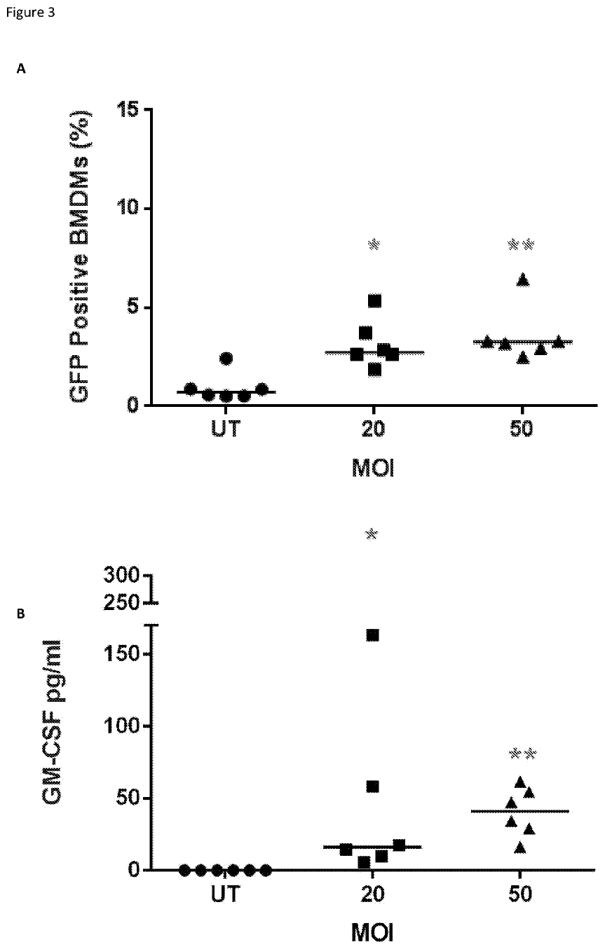

BMDM were transduced with F/HN pseudotyped simian immunodeficiency virus (SIV) expressing a) GFP or b) GM-CSF transgene at multiplicities of infection (MOI) of 20 or 50. Bone marrow was differentiated into macrophages by supplying M-CSF (100 ng/ml) in culture. Cells were transduced on day 7 (n=6/MOI) and then either: a) harvested on day 14 and the percentage of EGFP positive macrophages was determined by flow cytometry (BD Fortessa) using an extended antibody panel (CD45*, CD11b+, F4/80+, CD64+, and CD24) to identify bone marrow derived macrophages. b) or on day 16, GM-CSF levels were measured in 2-day old media using a mouse GM-CSF Quantikine ELISA kit (R&D systems). Media volume was controlled across samples. An MOI of 50 led to a marginal increase in GFP positive cells and GM-CSF production (FIG. 3).

### Example 3—BMDM are Unsuccessfully Transfected Using the Reagent PEI PRO

An attempt was made to transfect bone marrow derived macrophages to express a secreted reporter protein (Gaussia luciferase) using a commercially available (non-viral) transfection agent PEI Pro.

BMDM were transfected with a plasmid expressing Gaussia luciferase under the control of the hCEF promoter in a non-viral expression cassette. Bone marrow was differentiated into macrophages by supplying M-CSF (100 ng/ml) in culture and cells were transfected on day 7 (n=6/MOI). 48 hours post transfection, all media (1 ml per well) was removed and analysed using the Pierce™ Gaussia Luciferase Glow Assay Kit from Thermo Fischer Scientific. Media from A549 cells (adenocarcinomec human alveolar basal epithelial cells) transfected under the same protocol were used as a comparator.

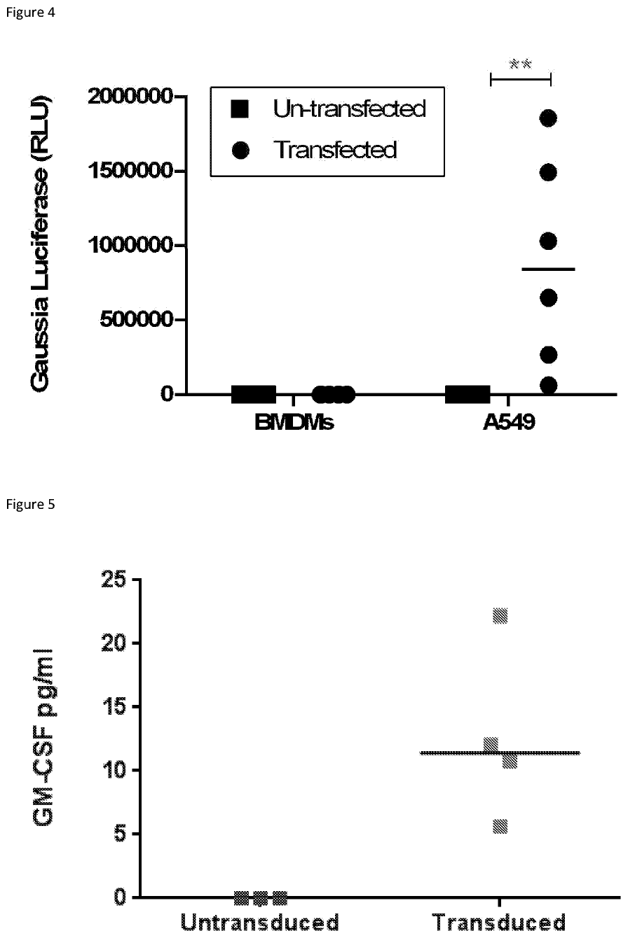

As shown in FIG. 4, there was no evidence of successful transfection or genetic modification of the macrophages. These data reinforce a consensus in the community that non-viral approaches to genetic modification of macrophages are unlikely to yield positive results.

### Example 4—BMDM Transduced with F/HN Lentivirus Secrete Detectable Levels of GM-CSF in the Mouse Lung

BMDM were transduced with F/HN lentivirus expressing GM-CSF under the control of the hCEF promoter at MOI 50. Bone marrow was differentiated into macrophages by supplying M-CSF (100 ng/ml) in culture and 9×10⁶ were transduced on day 7. 24 hours post transduction, cells were harvested, washed twice, and resuspended in PBS. 2×10⁶ BMDM were then delivered to the lungs of mice via oropharyngeal delivery in 50 μl of PBS. The BMDM were either transduced (n=4) or untransduced (n=3).

On day 7, mice were culled and bronchoalveolar lavage fluid (BALF) collected. GM-CSF concentrations were measured using a mouse GM-CSF Quantikine ELISA kit (R&D systems). Significant levels of GM-CSF in the BALF of mice were detected only when transduced BMDM were delivered (FIG. 5), demonstrating proof-of-concept for a macrophage based ex vivo cell therapy approach to expressing secreted proteins in the lung.

### Example 5—BMDM Transduced with F/HN Lentivirus Secrete Detectable Levels of Gaussia Luciferase (Gluc) in the Mouse Lung in a Dose Dependent Manner

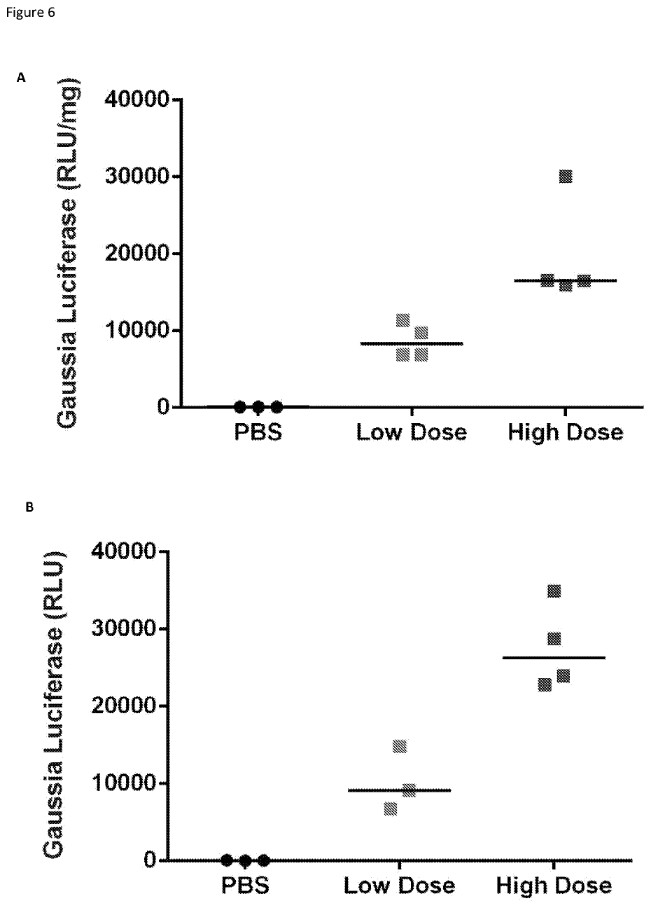

Bone marrow was differentiated into macrophages by supplying M-CSF (100 ng/ml) in culture. On day 7 BMDM were transduced with F/HN lentivirus expressing Gluc under the control of the hCEF promoter at MOI 20. 24 hours post transduction, cells were harvested, washed twice, and resuspended in PBS. Mice either received a low dose (2×10⁶) or high dose (6.32×10⁶) of transduced cells delivered to the mouse lung using an oropharyngeal delivery route. Control mice received cell-free PBS (n=4/group). BALF and lungs were collected 7 days post-delivery and Gluc levels were measured in 10 μl of lung homogenate (FIG. 6A) or BALF (FIG. 6B) using the Pierce™ Gaussia Luciferase Glow Assay Kit from Thermo Fischer Scientific. As shown in FIG. 6, dose-dependent Gluc secretion was observed in both the lung homogenate and BALF.

### Example 6—BMDM Transduced with F/HN Lentivirus Remain in the Mouse Lung and Secrete Detectable Levels of Gaussia Luciferase (Gluc) for Up to Two Weeks

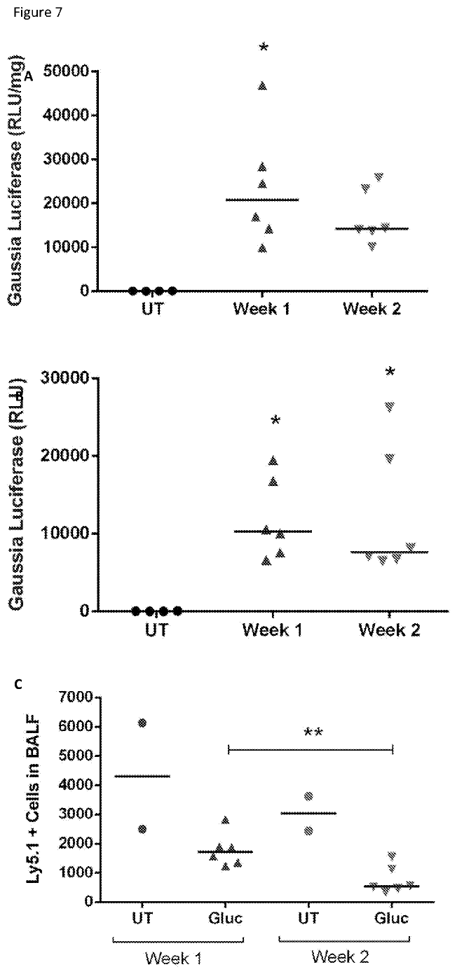

Bone marrow from a Ly5.1 mouse was differentiated into macrophages by supplying M-CSF (100 ng/ml) in culture. BMDM were transduced on day 7 with F/HN lentivirus expressing Gluc under the control of the hCEF promoter at MOI 20. 24 hours post transduction, cells were harvested, washed twice, and resuspended in PBS. 6.32×10⁶ transduced (n=6/group) or untransduced (UT) (n=2/group) cells were delivered to the lungs of Ly5.2 mice using an oropharyngeal delivery route. BALF and lungs were collected 1 and 2 weeks post-delivery and Gluc levels were measured in 10 μl of lung homogenate (FIG. 7A) or BALF (FIG. 7B) using the Pierce™ Gaussia Luciferase Glow Assay Kit from Thermo Fischer Scientific. As shown in FIGS. 7A and 7B, Gluc was expressed in the lung tissue and secreted into the BALF for at least two weeks following delivery of the modified BMDM.

The number of transplanted cells in the BALF was determined by staining for CD45.1 positive cells and the use of CountBright™ Absolute Counting Beads on a BD Accuri™ C6 flow cytometer (FIG. 7C). These data demonstrate that the transplanted BMDM are still present within the recipient mouse, as the cells themselves were directly identified using the donor-specific marker ly5.1. In other words, in addition to transgene expression being maintained, the transplanted BMDM were detected in the mouse lung at these two time points.

### Example 7—Bone Marrow Derived Macrophages (BMDM) Transduced with VSV-G Lentivirus Express a GFP Transgene at Promoter-Specific Levels of Intensity

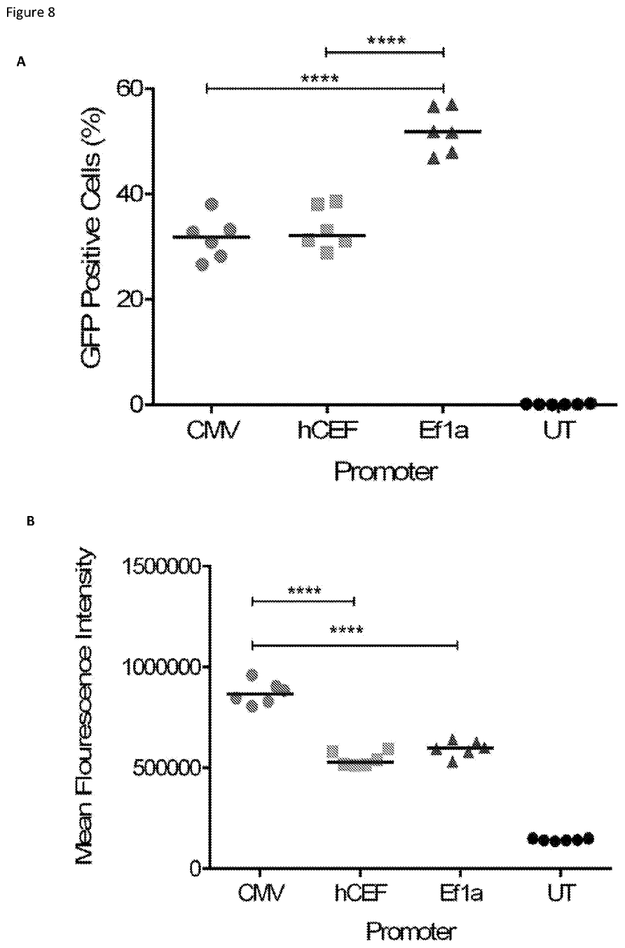

Bone marrow from C57BL/6 mice was differentiated into macrophages by supplying M-CSF (100 ng/ml) in culture. On day 7 (i.e. after cells were differentiated), BMDM were transduced with VSV-G pseudotyped simian immunodeficiency virus (SIV) expressing an EGFP transgene under the control of either a CMV, hCEF, or EF1a promoter at a multiplicity of infection of 50 (n=6/MOI). Cells were transduced on day 7 (i.e. after cells were differentiated), alongside untransduced (UT) controls (n=6). Cells were harvested 7 days post viral transduction and the percentage of EGFP positive macrophages (FIG. 8a), and the mean fluorescent intensity of those positive cells (FIG. 8b) was determined by flow cytometry (BD Accuri™ C6) using TO-PRO-3 to gate live cells, before analysing GFP positive cells. Ordinary One-Way ANOVA with Holm-Sidak's multiple comparisons test comparing promoter data sets (exl UT).

As can be seen from FIG. 8a, all three promoters were active, with >20% GFP positive cells being detected for each (EF1a achieving significantly higher GFP positive % cells than CMV or hCEF). FIG. 8b shows that all three promoters achieved increased GFP expression compared with the UT controls, with CMV achieving significantly greater fluorescence intensity than hCEF or EF1a.

### Example 8—Bone Marrow Derived Macrophages (BMDM) Transduced with F/HN Lentivirus to Secrete GM-CSF in the Mouse Lung, Lead to Robust Changes in Phenotypic Markers of Disease in Mouse Models of Pulmonary Alveolar Proteinosis (PAP)

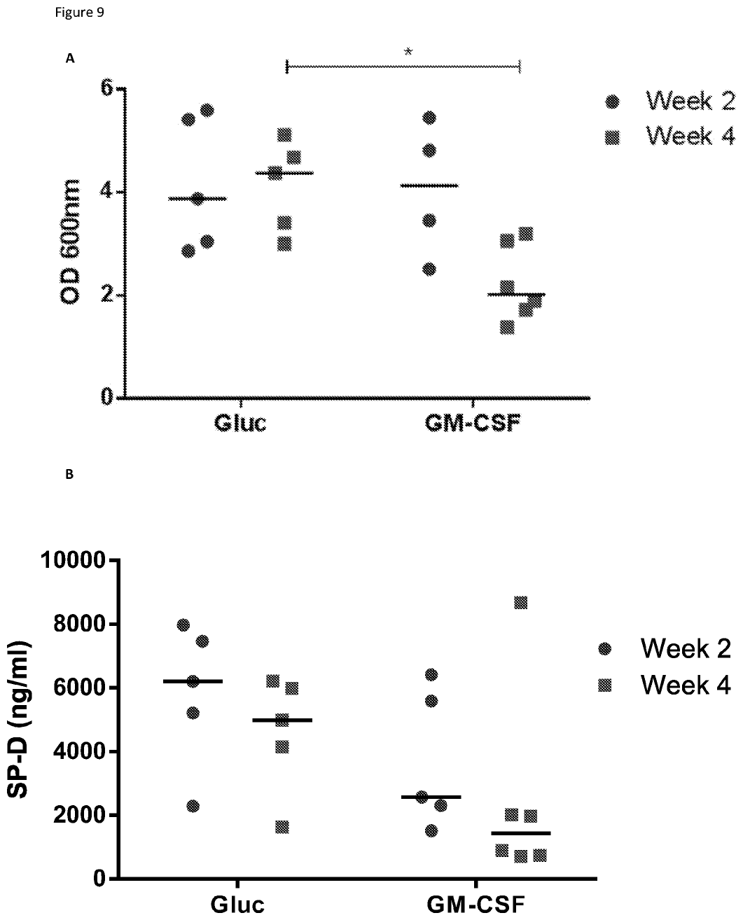

Bone marrow was differentiated into macrophages by supplying M-CSF (100 ng/ml) in culture and were transduced on day 7 with F/HN lentivirus expressing either GM-CSF (n=6/time point) or Gaussia luciferase (Gluc) (n=5/timepoint) under the control of the hCEF promoter at MOI 20. 24 hours post transduction, cells were harvested, washed twice, and resuspended in PBS. 6.32×10⁶ transduced cells were delivered to the lungs of GM-CSF knock-out mice which are a model of PAP, using an oropharyngeal delivery route. BALF was collected 2 weeks or 4 weeks post-delivery and the turbidity of mouse BALF was read by measuring the optical density (OD) of the BALF at 600 nm (FIG. 9A), and the concentration of SP-D in BALF was quantified using a Mouse SP-D Quantikine ELISA Kit from R&D Systems (FIG. 9B).

The BALF of PAP patients is characterised by a milky/white turbid appearance, thick sediment and accumulation of surfactant protein D (SP-D), which is recapitulated in this mouse model of PAP disease. As can be seen in FIG. 9, as compared to expression of the Gluc reporter gene, in vivo expression of GM-CSF following macrophage delivery leads to robust changes in phenotypic markers of PAP disease as four-weeks post-delivery BALF showed marked decrease in turbidity (FIG. 9A) and an observable trend toward a reduced SP-D burden.

### Example 9—BMDM Transduced with F/HN Lentivirus Exhibit Sustained Transgene Expression In Vivo Following Transplantation

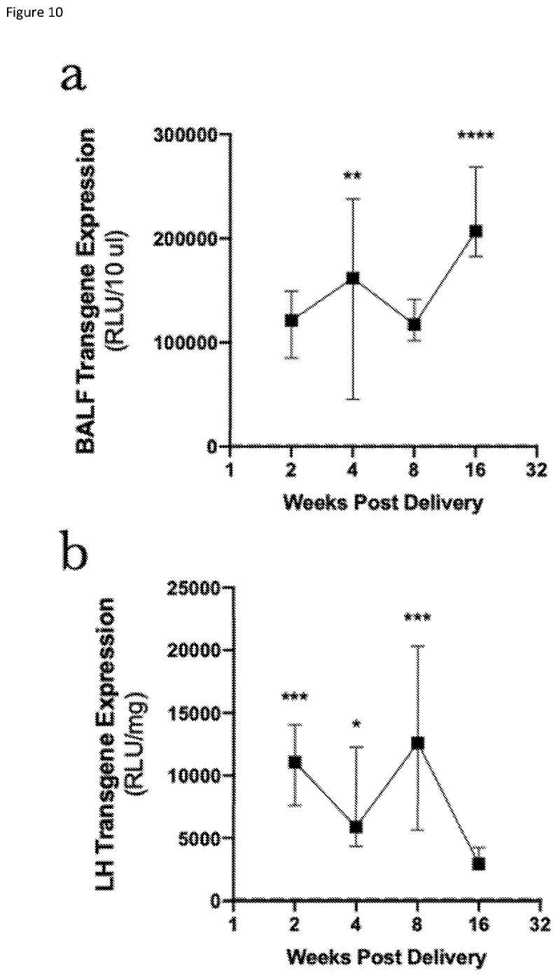

BMDMs were transduced ex vivo with rSIV.F/HN lentivirus encoding murine Glux under the control of the hCEF promoter at MOI 20. Following a single dose of 3×10⁶ cells delivered via oropharyngeal delivery Glux expression was detected in bronchoalveolar lavage fluid (BALF) (FIG. 10A) and lung homogenate (LH) (FIG. 10B) 2, 4, 8, and 16 weeks later

Glux expression was compared to samples from control mice which received a corresponding dose of un-transduced macrophages and were harvested at week 2 (n=6), maximum reported Glux expression in these mice is indicated by red dotted line in FIGS. 10A and B. Maximum expression shadows the x-axis and was essentially 0 in both BALF and LH.

Glux was quantified in 10 μl of BALF and lung homogenate, the latter were then normalised to total protein concentration (mg/ml).

### Example 10—BMDMs are Retained in the Lung Following Transplantation

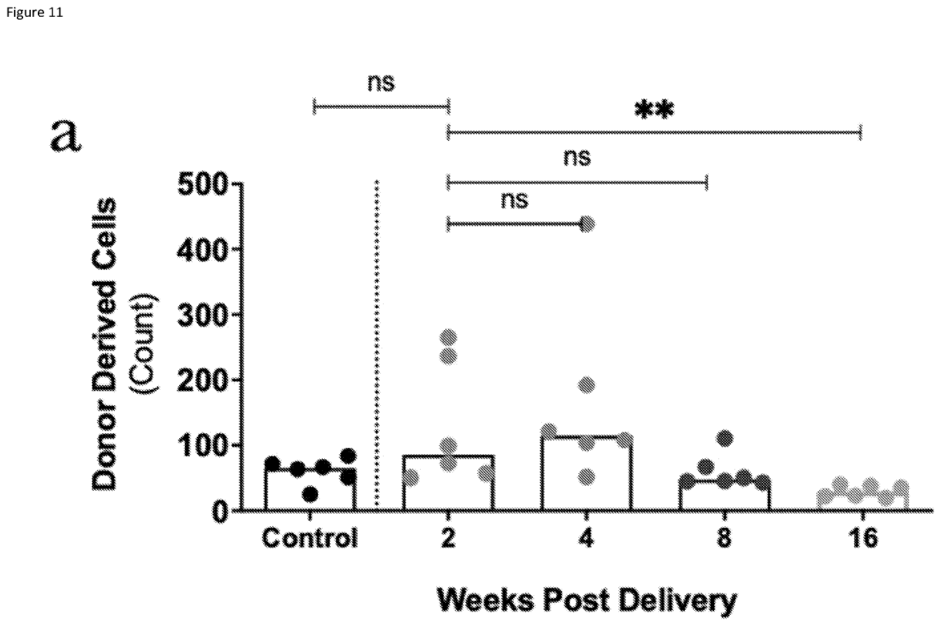

A single dose of 3×10⁶ BMDMs transduced ex vivo with rSIV.F/HN.hCEF.Glux were delivered via oropharyngeal delivery to WT mice. The number of donor-derived cells (CD45.1⁺) in BALF samples were identified by flow cytometry in the BALF of mice harvested 2, 4, 8, and 16 weeks post-delivery (n=6/group). Absolute counts of donor-derived cells in BALF were extrapolated using counting beads. The number of donor-derived cells remained statistically unchanged in samples taken at 2, 4 and even 8 weeks, with a statistically significant reduction only being observed 16 weeks post-transplantation (FIG. 11).

### Example 11—Similar Transduction Efficiency and Integration in BMDMs is Achieved Using Both VSV-G and F/HN Pseudotyped rSIV Lentivirus

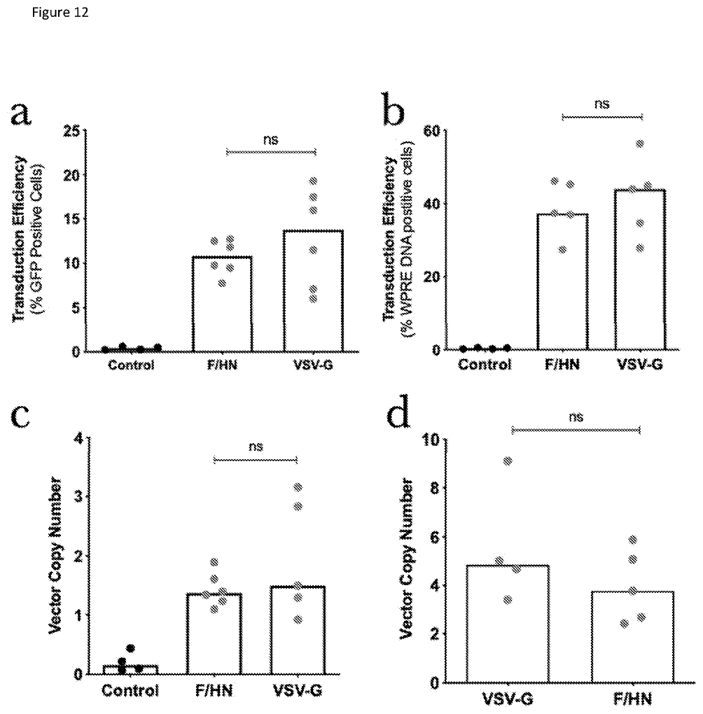

Comparisons between rSIV vectors with a VSV-G and F/HN pseudotype in BMDMs transduced at MOI 46. Untransduced cells served as control samples. Transduction efficiency was calculated by flow cytometry analysis of EGFP transgene expression (n=6) (FIG. 12A), and a ddPCR encapsulation assay quantifying cells positive for viral cDNA (n=5) (FIG. 12B). Vector copy number (VCN) ddPCR analysis of reverse transcribed lentiviral genomes was performed from bulk extracted DNA samples (n=6) (FIG. 12C), and then corrected for transduction efficiency as quantified by the encapsulation assay (FIG. 12D).

BMDMs were transduced for 24 hours and then analysed on day 7. Each sample was split into thirds, 1/3 was then analysed by flow cytometry, another 1/3 by encapsulation. The final 1/3 was divided equally into two and used to procure RNA and DNA extractions. Flow cytometry capture by BD Accuri™ C6 Plus Flow Cytometer, analysis in FlowJo software. Each data point represents an individually transduced well of BMDMs.

Using all metrics, there was no statistical difference in transduction efficiency or integration between VSV-G and F/HN rSIV vectors in BMDMs.

### Example 12—Transgene mRNA and Vector Specific RNA Comparisons in BMDMs Transduced with VSV-G and F/HN Pseudotyped rSIV Lentivirus

The concentration of RRE (left data set for each transfection condition) and WPRE (right data set for each transfection condition) positive RNA copies were quantified by ddPCR in BMDMs transduced by rSIV vectors with a VSV-G or F/HN pseudotype (MOI 46). Untransduced cells served as control. RNA analysis performed on bulk extracted samples of RNA, where RRE is specific to viral genomes, and WPRE is present on both viral genomes and transgene mRNA.

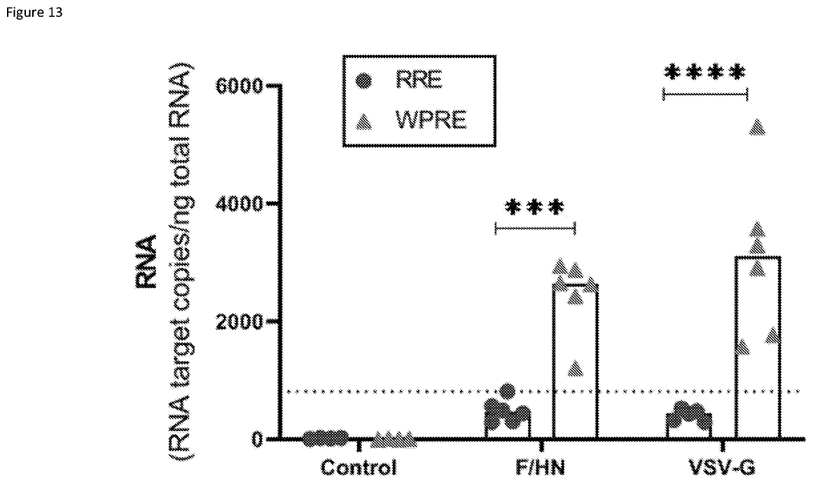

BMDMs were transduced for 24 hours and then analysed on day 7. Each sample was split into thirds, 1/3 was then analysed by flow cytometry, another 1/3 by encapsulation. The final 1/3 was divided equally into two and used to procure RNA and DNA extractions. For both VSV-G and F/HN rSIV, the concentration of WPRE was significantly greater than that of RRE (FIG. 13), demonstrating efficient expression of transgene mRNA.

### Example 13—Relative Transduction Efficiency of rSIV Vectors Encoding CMV, hCEF, and EF1a Promoters in BMDMs

The relative transduction efficiency of BMDMs following transduction with rSIV.VSV-G vectors expressing an EGFP transgene under the control of the CMV, EF1a, and hCEF promoters at increasing multiplicities of infection (MOI: 10, 20, 50) (n=6/MOI). Transduction efficiency was calculated as the percentage of EGFP positive cells in culture by flow cytometry 7 days post-initiation of transduction (48 hours). Capture by BD Accuri™ C6 Plus Flow Cytometer.

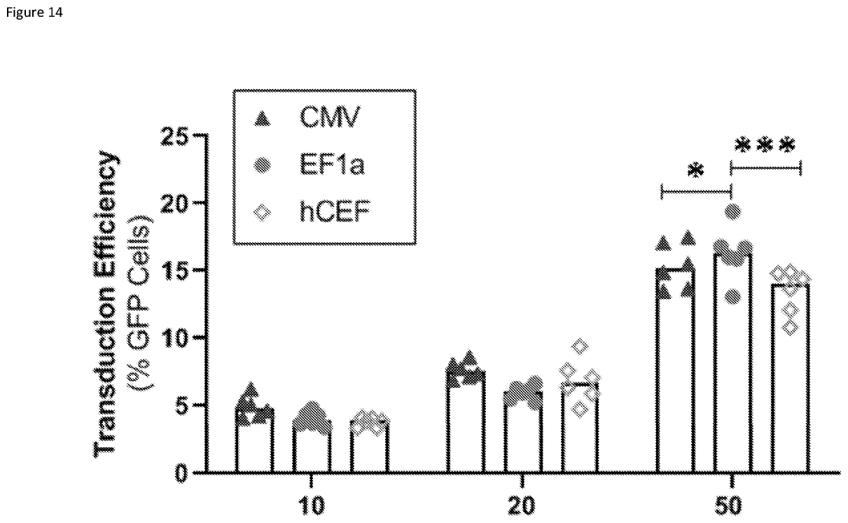

For each promoter, greatest transduction efficiency was observed at MOI 50. At this MOI, the EF1a promoter achieved statistically greater transduction efficiency (FIG. 14).

### Example 14—Relative Gene Expression of CMV, hCEF, and EF1a Promoters in BMDMs

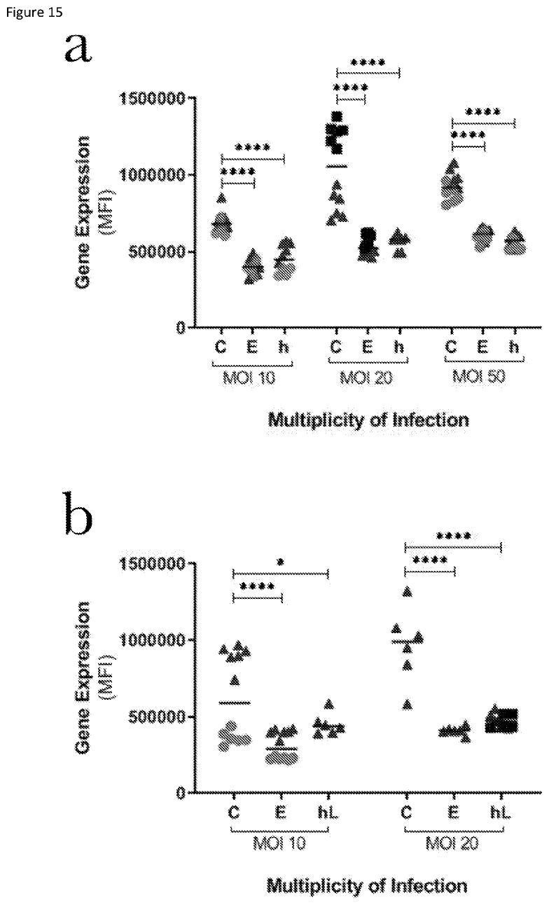

BMDMs were transduced with rSIV.VSV-G (FIG. 15A) and rSIV.F/HN (FIG. 15B) vectors expressing an EGFP transgene under the control of the CMV (C), EF1a (E), and hCEF (h) promoters at increasing multiplicities of infection (MOI: 10, 20, 50) (n=6). Gene expression quantified as mean fluorescent intensity (MFI) of EGFP expression by flow cytometry across previous transduction experiments. Colours relate to different experiments, black square: experiment 5.3.4; red circle: experiment 5.3.5; blue triangle: experiment 5.3.6. Capture by BD Accuri™ C6 Plus Flow Cytometer. Each data point represents an individually transduced well of BMDMs. Bars represent median. All crude vectors produced in small batches, except the large scale purified rSIV.F/HN.hCEF vector in experiment 5.3.4 (hL).

For both rSIV.VSV-G and rSIV.F/HN, greatest gene expression was achieved using the CMV promoter at all MOI.

### Example 15—Induction of Gaussia Luciferase (Glux) Transgene Expression in BMDMs

BMDMs were transduced with one of two regulated expression lentiviral systems: a dual (2) vector system (commercially available GeneSwitch) or a single vector system (derived from GeneSwitch) for 24 hours (+SIV) (MOI 10—controlled for Glux encoding vector). Gene expression was induced by a 24-hour incubation with 10 UM of mifepristone (+M), and Glux was quantified in cell culture media every 24 hours for 5 days, where at each time point cells were incubated with fresh media (1 ml).

Expression in induced samples was compared to non-induced controls at each time point to determine the degree of induction for each vector system. Positive controls were transduced with rSIV.F/HN.Glux whose expression steadily increased over time and untransduced cells served as negative controls.

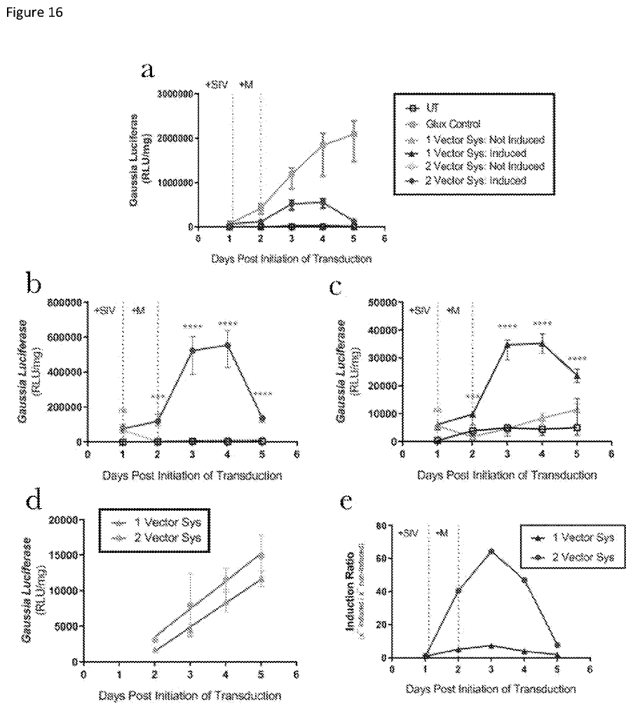

The dual vector system achieved significant expression from 2 days post initiation of transduction, which peaked at day 3-4, with significant transduction still observed at day 5 (FIG. 16B). A similar time course of expression was observed using the single vector system (FIG. 16C). Untransduced controls exhibited increasing levels of leaky expression over time, each data set was fit to a model of simple linear regression (FIG. 16D). The induction ratio of each vector system was calculated as the median difference in gene expression between induced and non-induced controls. Glux was quantified as relative light units (RLU) in 10 μl of media by Pierce™ Gaussia Luciferase Glow Assay Kit and normalised by cell number at the end of the experiment by quantification of total protein (mg/ml) (FIG. 16E). The results of all test conditions and controls were plotted on one graph (FIG. 16A).

### Example 16—Induction of Gaussia Luciferase (Glux) Transgene Expression in BMDMs

BMDMs were transduced with one of two regulated expression lentiviral systems: a dual (2) vector system (commercially available GeneSwitch) or a single vector system (derived from GeneSwitch) for 24 hours (+SIV) (MOI 10-controlled for Glux encoding vector). Gene expression was induced by a 24-hour incubation with 10 UM of mifepristone (+M) on day 2 and day 6. Glux was quantified in cell culture media every 24 hours for up to 7 days post transduction, where at each time point cells were incubated with fresh media (1 ml).

Expression in induced samples was compared to non-induced controls at each time point to determine the degree of induction for each vector system. Positive controls were transduced with rSIV.F/HN.Glux whose expression steadily increased over time and untransduced cells served as negative controls.

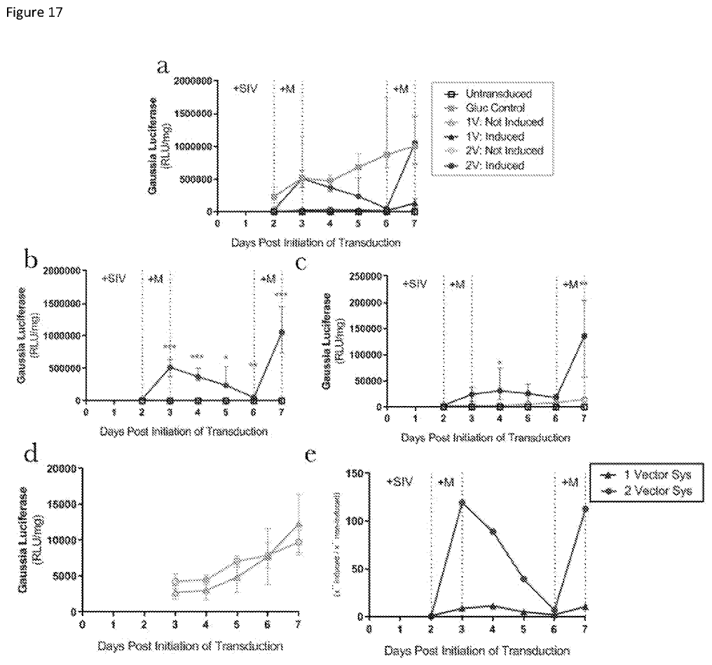

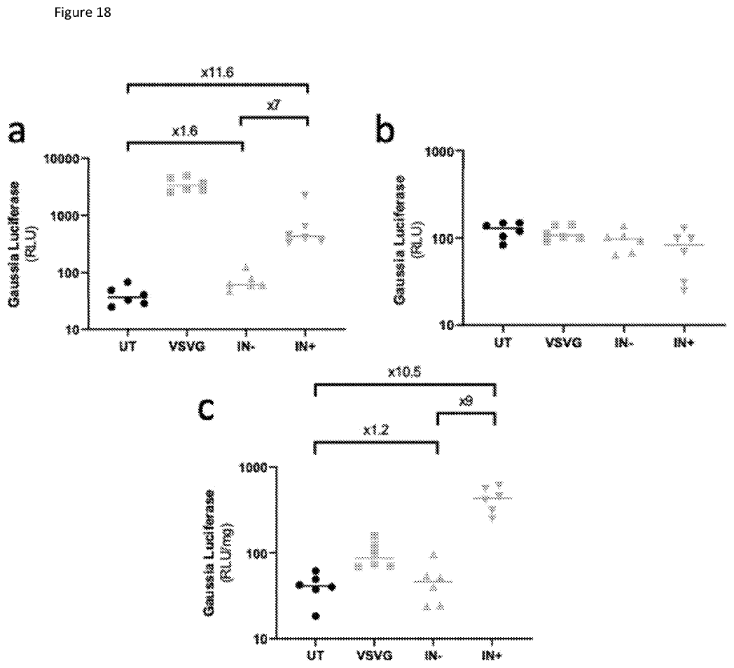

The dual vector system achieved significant expression from 3 days post the first (day 2) initiation of transduction, which decreased from days 4-6 (but still significant), with a greater increase in expression observed on day 7 following the second initiation on day 6 (FIG. 17B). The single vector system achieved significant expression on day 4 post the first (day 2) initiation of transduction, with a greater increase in expression observed on day 7 following the second initiation on day 6 (FIG. 17C). Untransduced controls exhibited increasing levels of leaky expression over time (FIG. 17D). The induction ratio of each vector system was calculated as the median difference in gene expression between induced and non-induced controls. Glux was quantified as relative light units (RLU) in 10 μl of media by Pierce™ Gaussia Luciferase Glow Assay Kit and normalised by cell number at the end of the experiment by quantification of total protein (mg/ml) (FIG. 17E). The results of all test conditions and controls were plotted on one graph (FIG. 17A).

### Example 17—In Vivo Induction of Gaussia Luciferase (Glux) Transgene Expression from Lung Transplanted BMDMs

Bone marrow cells were transduced during differentiation into macrophages (Day 0-2; 48 hours) with a VSV-G-hCEF-Glux constitutive expression vector (VSV-G) or a dual vector regulated expression lentiviral system (MOI 20-controlled for Glux encoding vector). 1E6 cells/mouse were transplanted via oropharyngeal delivery to the lungs of wild type mice as differentiated macrophages on day 7. Mice either received untransduced cells (UT, n=6), cells transduced with VSV-G (n=6), or the inducible vector system (IN, n=12). 7 days later, n=6 of IN mice (IN+) and UT controls received daily administration of 0.5 mg/kg of mifepristone by IP injection for four days. The remaining n=6 IN mice did not receive mifepristone (IN−) and represent test conditions where Glux expression was not induced. All mice were harvested 12 days after cell delivery: the day following the final administration of mifepristone. Glux was quantified as relative light units (RLU) by Pierce™ Gaussia Luciferase Glow Assay Kit in 15 μl of (a) BALF, (b) Serum and (c) lung homogenate, the latter were then normalised to total protein concentration (mg/mL).

Glux expression in BALF from mice which received IN cells and were induced with mifepristone was 7-fold greater than in mice which received IN cells but which were not induced with mifepristone. Glux expression in BALF from mice which received IN cells and were induced with mifepristone was 11.6-fold greater than in BALF from mice which received untransduced cells.

Glux expression in the lung homogenate of mice which received IN cells and were induced with mifepristone was 9-fold greater than in mice which received IN cells but which were not induced with mifepristone. Glux expression in the lung homogenate of mice which received IN cells and were induced with mifepristone was 10.5-fold greater than in lung homogenate of mice which received untransduced cells. No significant difference in Glux expression was observed in the serum of the different groups.

These results demonstrate that transgene expression in macrophages may be driven in vivo using a commercial regulated promoter system (GeneSwitch).

1. An ex vivo method for obtaining immune cells, immunomodulatory cells, induced pluripotent stem cells (iPSCs) or iPSC-derived cells modified to express a transgene of interest, said method comprising transducing the cells with a lentiviral vector comprising the transgene, wherein the transgene is a secreted therapeutic protein.

2. The method according to claim 1, wherein the lentiviral vector is pseudotyped with haemagglutinin-neuraminidase (HN) and fusion (F) proteins from a respiratory paramyxovirus or G glycoprotein from Vesicular Stomatitis Virus (G-VSV).

3. The method according to claim 1, wherein the secreted therapeutic protein is selected from Alpha-1 Antitrypsin (A1A1), Factor VIII, Surfactant Protein B (SFTPB), Factor VII, Factor IX, Factor X, Factor XI, van Willebrand Factor, Granulocyte-Macrophage Colony-Stimulating Factor (GM-CSF), decorin, an anti-inflammatory protein or monoclonal antibody, and a monoclonal antibody against an infectious agent.

4. An ex vivo method for obtaining immune cells, immunomodulatory cells, iPSC, or iPSC-derived cells modified to express a transgene of interest, said method comprising transducing the cells with a lentiviral vector comprising the transgene, wherein the lentiviral vector is pseudotyped with haemagglutinin-neuraminidase (HN) and fusion (F) proteins from a respiratory paramyxovirus or G glycoprotein from Vesicular Stomatitis Virus (G-VSV).

5. The method according to claim 4, wherein the transgene encodes a non-secreted protein involved in macrophage biology selected from CFTR, CSF2RA, CSF2RB, and TRIM-72.

6. The method according to claim 1, wherein the lentiviral vector is selected from the group consisting of a Human immunodeficiency virus (HIV) vector, a Simian immunodeficiency virus (SIV) vector, a Feline immunodeficiency virus (FIV) vector, an Equine infectious anaemia virus (EIAV) vector, and a Visna/maedi virus vector.

7. The method according to claim 6, wherein the lentiviral vector is a SIV vector.

8. The method according to claim 1, wherein the respiratory paramyxovirus is a Sendai virus.

9. The method according to claim 1, wherein the vector further comprises a promoter selected from the group consisting of: a hybrid human cytomegalovirus (CMV) enhancer/elongation factor 1 a (EF1 a) promoter (hCEF), a CMV promoter, an EF1 a promoter, and a steroid-regulated promoter.

10. The method according to claim 1, wherein the immune cells, immunomodulatory cells, iPSC, or iPSC-derived cells are derived from peripheral blood, cord blood, bone marrow, fibroblasts, or adipose tissue.

11. The method according to claim 1, wherein the immune cells, immunomodulatory cells, iPSC, or iPSC-derived cells are:
(a) immune cells which are peripheral blood mononuclear cells (PBMCs);
(b) immunomodulatory cells; or
(c) iPSCs or iPSC-derived cells.

12. The method according to claim 1, wherein the immune cells, immunomodulatory cells, iPSC, or iPSC-derived cells are:
(a) differentiated macrophages;
(b) monocytes, iPSCs or MSCs, and which are differentiated to macrophages after the cells have been transduced with the lentiviral vector; or
(c) iPSC-derived cells which are differentiated from iPSC prior to transduction with the lentiviral vector.

13. The method according to claim 1, wherein the immune cells, immunomodulatory cells, iPSC, or iPSC-derived cells (i) are naïve; or (ii) have previously been mobilized.

14. The method according to claim 1, further comprising:
(a) a step of expanding the immune cells, immunomodulatory cells, iPSC, or iPSC-derived cells prior to transduction with the lentiviral vector; or
(b) a step of expanding the immune cells, immunomodulatory cells, iPSC, or iPSC-derived cells after transduction with the lentiviral vector.

15. The method according to claim 1, further comprising one or more steps to isolate and/or concentrate the immune cells, immunomodulatory cells, iPSC, or iPSC-derived cells, wherein the one or more steps are carried out:
(a) prior to or after the immune cells, immunomodulatory cells, iPSC, or iPSC-derived cells are transduced with the lentiviral vector; and/or
(b) prior to or after a step of expanding the immune cells, immunomodulatory cells, iPSC, or iPSC-derived cells.

16. A population of modified immune cells, immunomodulatory cells, iPSC,
or iPSC-derived cells comprising a transgene of interest, which immune cells, immunomodulatory cells, iPSC, or iPSC-derived cells are obtainable by a method as defined in claim 1.

17. (canceled)

18. (canceled)

19. (canceled)

20. (canceled)

21. (canceled)

22. (canceled)

23. A composition comprising a population of modified immune cells, immunomodulatory cells, iPSCs or iPSC-derived cells comprising a transgene of interest as defined in claim 16, and a pharmaceutically acceptable excipient, buffer, or diluent.

24. (canceled)

25. A gene therapy method comprising administering to a subject in need thereof a therapeutically effective amount of a population of modified immune cells, immunomodulatory cells, iPSCs or iPSC-derived cells comprising a transgene of interest, which cells are obtainable by a method as defined in claim 1.

26. The gene therapy method of claim 25, wherein the immune cells, immunomodulatory cells, iPSCs or iPSC-derived cells are:
(a) autologous cells derived from a patient to be treated; or
(b) allogenic cells derived from an individual other than the patient.

27. The gene therapy method of claim 25, wherein said gene therapy is for the treatment of Cystic Fibrosis (CF); Alpha 1-antitrypsin Deficiency (A1AD); Pulmonary Alveolar Proteinosis (PAP); Chronic obstructive pulmonary disease (COPD); a surfactant deficiency; an inflammatory or allergic lung condition; an infection of the lung; lung cancer; asthma or a fibrotic lung condition.


SEQUENCE LISTING (27 sequences)

SEQ ID NO: 1 - DNA, 574 bp, Artificial Sequence
  hCEF promoter
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagc

SEQ ID NO: 2 - DNA, 873 bp, Cauliflower mosaic virus
ccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagc

SEQ ID NO: 3 - DNA, 395 bp, Homo sapiens
agatccatatccgcggcaattttaaaagaaagggaggaatagggggacagacttcagcagagagactaattaatataataacaacacaattagaaatacaacatttacaaaccaaaattcaaaaaattttaaattttagagccgcggagatcccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagc

SEQ ID NO: 4 - DNA, 4443 bp, Artificial Sequence
  soCFTR2
atgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtga

SEQ ID NO: 5 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 6 - DNA, 435 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 7 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 8 - DNA, 426 bp, Mus musculus
atgtggctgcagaacctgctgttcctgggcattgtggtgtacagcctgtctgcccctacaagatcccctatcacagtgaccagaccttggaaacatgtggaagccatcaaagaggccctgaatctgctggatgacatgcctgtgacactgaatgaagaggtggaagtggtgtccaatgagttcagcttcaagaaactgacctgtgtgcagaccaggctgaagatttttgagcagggcctgagaggcaacttcaccaagctgaaaggggctctgaacatgacagccagctactaccagacctactgtcctcctacacctgagacagactgtgaaacccaagtgaccacctatgctgacttcattgacagcctcaagaccttcctgacagacatcccctttgagtgcaagaaacctggccagaagtga

SEQ ID NO: 9 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 10 - DNA, 1257 bp, Homo sapiens
atgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatga

SEQ ID NO: 11 - DNA, 1257 bp, Homo sapiens
tacgggtcgagacacaggaccccgtaagacgacgaccgaccggacacgacagaccacggacacagggaccgactcctgggagtccccctacgacgggtcttttgtctgtggagggtggtactggtcctggtggggtggaagttgttctagtgggggttggaccgtctcaaacggaagtcggacatgtctgtcgaccgggtggtctcgttgtcgtggttgtagaaaaagtcgggacacaggtaacggtgtcggaaacggtacgactcggacccgtggttccgactgtgggtactactctaggaccttccggacttgaagttggactgtctctagggactccgggtctaggtactcccgaaggtccttgacgactcttgggacttggtcggtctgtcggtcgacgtcgactgttgtccgttacccgacaaggacagactcccggacttcgaccacctgttcaaagaccttctacacttcttcgacatggtgagactccggaagtgtcacttgaaacccctgtgtcttctccggttctttgtctagttactgatacaccttttcccgtgggtcccgttctaacacctggaacactttctcgacctgtccctgtgacacaaacgggaacacttgatgtagaagaagttcccgttcaccctctccgggaaacttcacttcctgtgactccttctcctgaaggtacacctggttcactggtgtcacttccacggttactacttctctgacccctacaagttataggtcgtgacgttctttgactcgtcgacccacgacgactacttcatggacccgttacgatgtcggtataagaaagacggactactcccgttcgacgtcgtggaccttttactcgactgggtactgtagtagtggtttaaagaccttttactcctgtcttctagacggtcggacgtagacgggttcgactcgtagtgtccgtgtatactggacttcagacacgaccctgtcgacccttagtggttccacaagtcgttaccccgtctggactcacctcactgtctccttcggggagacttcgacaggttccgacacgtgttccgtcacgactggtaactactcttcccgtgtctccgacgaccccggtacaaagaccttcggtaggggtacaggtaggggggtcttcacttcaagttgttcgggaaacacaaggactactaactcgtcttgtggttctcgggggacaagtacccgttccaacacttggggtgggtctttact

SEQ ID NO: 12 - protein, 442 aa, Homo sapiens
MPSSVSWGILLLAGLCCLVPVSLAEDPQGDAAQKTDTSHHDQDHPTFAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 13 - DNA, 5013 bp, Artificial Sequence
  FVIII N6 transgene
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 14 - DNA, 4425 bp, Artificial Sequence
  FVIII V3 transgene
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 15 - DNA, 5013 bp, Artificial Sequence
  Complementary strand to FVIII transgene (N6)
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttgtcgtccgtggggtcgtggtccgtcttcgtcaagttacggtggtggtagggactcttactgtatctcttctgtctgggtaccaaacgggtggcctgggggtacgggttctaggtcttacactcgtcgagactggacgactacgacgactccgtctcggggtggggggtaccggactcggacagactggacgtcctccggttcatactttggaagagactactggggtcgggaccccggtaactgtcgttgttgtcggacagactctactgggtgaagtccggggtcgacgtggtgagacccctgtaccacaagtggggactcagaccggacgtcgactccgacttactcttcgacccgtggtgacgacggtgactcgacttcttcgacctgaagtttcagaggtcgtggtcgttgttggactagtcgtggtaggggagactgttggaccgacgaccgtgactgttgtggtcgtcggacccgggggggtcgtacggacacgtgatactgtcggtcgacctgtggtgggacaaaccgttcttctcgtcgggggactgactcagacccccgggggactcggacagactcctcttgttactgtcgttcgacgacctcagaccggactacttgtcggtcctctcgtcgaccccgttcttacactcgtcgtccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 16 - DNA, 4425 bp, Artificial Sequence
  Complementary strand to FVIII transgene (V3)
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttacggtgattacacagattgttgtcgttgtggtcgttactgtcgttacacagagggggtcacgacttctccgtggtctccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 17 - protein, 1670 aa, Artificial Sequence
  FVIII polypeptide (N6)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 18 - protein, 1474 aa, Artificial Sequence [sequence omitted]

SEQ ID NO: 19 - DNA, 1080 bp, Homo sapiens
atgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaa

SEQ ID NO: 20 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 21 - DNA, 1434 bp, Homo sapiens
atgtcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgcaaggagaagagtgtggctgtgctggagcatcagctggtggaggtggaggagacagtgcgtcagttccggggggccgtgggggagcagctgggcaagatgcgggtgttcctggctgcactggagggctccttggaccgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcctga

SEQ ID NO: 22 - protein, 477 aa, Homo sapiens
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 23 - DNA, 5115 bp, Homo sapiens
atggctgtgctcaggcagctggcgctcctcctctggaagaactacaccctgcagaagcggaaggtcctggtgacggtcctggaactcttcctgccattgctgttttctgggatcctcatctggctccgcttgaagattcagtcggaaaatgtgcccaacgccaccatctacccgggccagtccatccaggagctgcctctgttcttcaccttccctccgccaggagacacctgggagcttgcctacatcccttctcacagtgacgctgccaagaccgtcactgagacagtgcgcagggcacttgtgatcaacatgcgagtgcgcggctttccctccgagaaggactttgaggactacattaggtacgacaactgctcgtccagcgtgctggccgccgtggtcttcgagcaccccttcaaccacagcaaggagcccctgccgctggcggtgaaatatcacctacggttcagttacacacggagaaattacatgtggacccaaacaggctcctttttcctgaaagagacagaaggctggcacactacttcccttttcccgcttttcccaaacccaggaccaagggaacctacatcccctgatggcggagaacctgggtacatccgggaaggcttcctggccgtgcagcatgctgtggaccgggccatcatggagtaccatgccgatgccgccacacgccagctgttccagagactgacggtgaccatcaagaggttcccgtacccgccgttcatcgcagaccccttcctcgtggccatccagtaccagctgcccctgctgctgctgctcagcttcacctacaccgcgctcaccattgcccgtgctgtcgtgcaggagaaggaaaggaggctgaaggagtacatgcgcatgatggggctcagcagctggctgcactggagtgcctggttcctcttgttcttcctcttcctcctcatcgccgcctccttcatgaccctgctcttctgtgtcaaggtgaagccaaatgtagccgtgctgtcccgcagcgacccctccctggtgctcgccttcctgctgtgcttcgccatctctaccatctccttcagcttcatggtcagcaccttcttcagcaaagccaacatggcagcagccttcggaggcttcctctacttcttcacctacatcccctacttcttcgtggcccctcggtacaactggatgactctgagccagaagctctgctcctgcctcctgtctaatgtcgccatggcaatgggagcccagctcattgggaaatttgaggcgaaaggcatgggcatccagtggcgagacctcctgagtcccgtcaacgtggacgacgacttctgcttcgggcaggtgctggggatgctgctgctggactctgtgctctatggcctggtgacctggtacatggaggccgtcttcccagggcagttcggcgtgcctcagccctggtacttcttcatcatgccctcctattggtgtgggaagccaagggcggttgcagggaaggaggaagaagacagtgaccccgagaaagcactcagaaacgagtactttgaagccgagccagaggacctggtggcggggatcaagatcaagcacctgtccaaggtgttcagggtgggaaataaggacagggcggccgtcagagacctgaacctcaacctgtacgagggacagatcaccgtcctgctgggccacaacggtgccgggaagaccaccaccctctccatgctcacaggtctctttccccccaccagtggacgggcatacatcagcgggtatgaaatttcccaggacatggttcagatccggaagagcctgggcctgtgcccgcagcacgacatcctgtttgacaacttgacagtcgcagagcacctttatttctacgcccagctgaagggcctgtcacgtcagaagtgccctgaagaagtcaagcagatgctgcacatcatcggcctggaggacaagtggaactcacggagccgcttcctgagcgggggcatgaggcgcaagctctccatcggcatcgccctcatcgcaggctccaaggtgctgatactggacgagcccacctcgggcatggacgccatctccaggagggccatctgggatcttcttcagcggcagaaaagtgaccgcaccatcgtgctgaccacccacttcatggacgaggctgacctgctgggagaccgcatcgccatcatggccaagggggagctgcagtgctgcgggtcctcgctgttcctcaagcagaaatacggtgccggctatcacatgacgctggtgaaggagccgcactgcaacccggaagacatctcccagctggtccaccaccacgtgcccaacgccacgctggagagcagcgctggggccgagctgtctttcatccttcccagagagagcacgcacaggtttgaaggtctctttgctaaactggagaagaagcagaaagagctgggcattgccagctttggggcatccatcaccaccatggaggaagtcttccttcgggtcgggaagctggtggacagcagtatggacatccaggccatccagctccctgccctgcagtaccagcacgagaggcgcgccagcgactgggctgtggacagcaacctctgtggggccatggacccctccgacggcattggagccctcatcgaggaggagcgcaccgctgtcaagctcaacactgggctcgccctgcactgccagcaattctgggccatgttcctgaagaaggccgcatacagctggcgcgagtggaaaatggtggcggcacaggtcctggtgcctctgacctgcgtcaccctggccctcctggccatcaactactcctcggagctcttcgacgaccccatgctgaggctgaccttgggcgagtacggcagaaccgtcgtgcccttctcagttcccgggacctcccagctgggtcagcagctgtcagagcatctgaaagacgcactgcaggctgagggacaggagccccgcgaggtgctcggtgacctggaggagttcttgatcttcagggcttctgtggaggggggcggctttaatgagcggtgccttgtggcagcgtccttcagagatgtgggagagcgcacggtcgtcaacgccttgttcaacaaccaggcgtaccactctccagccactgccctggccgtcgtggacaaccttctgttcaagctgctgtgcgggcctcacgcctccattgtggtctccaacttcccccagccccggagcgccctgcaggctgccaaggaccagtttaacgagggccggaagggattcgacattgccctcaacctgctcttcgccatggcattcttggccagcacgttctccatcctggcggtcagcgagagggccgtgcaggccaagcatgtgcagtttgtgagtggagtccacgtggccagtttctggctctctgctctgctgtgggacctcatctccttcctcatccccagtctgctgctgctggtggtgtttaaggccttcgacgtgcgtgccttcacgcgggacggccacatggctgacaccctgctgctgctcctgctctacggctgggccatcatccccctcatgtacctgatgaacttcttcttcttgggggcggccactgcctacacgaggctgaccatcttcaacatcctgtcaggcatcgccaccttcctgatggtcaccatcatgcgcatcccagctgtaaaactggaagaactttccaaaaccctggatcacgtgttcctggtgctgcccaaccactgtctggggatggcagtcagcagtttctacgagaactacgagacgcggaggtactgcacctcctccgaggtcgccgcccactactgcaagaaatataacatccagtaccaggagaacttctatgcctggagcgccccgggggtcggccggtttgtggcctccatggccgcctcagggtgcgcctacctcatcctgctcttcctcatcgagaccaacctgcttcagagactcaggggcatcctctgcgccctccggaggaggcggacactgacagaattatacacccggatgcctgtgcttcctgaggaccaagatgtagcggacgagaggacccgcatcctggcccccagtccggactccctgctccacacacctctgattatcaaggagctctccaaggtgtacgagcagcgggtgcccctcctggccgtggacaggctctccctcgcggtgcagaaaggggagtgcttcggcctgctgggcttcaatggagccgggaagaccacgactttcaaaatgctgaccggggaggagagcctcacttctggggatgcctttgtcgggggtcacagaatcagctctgatgtcggaaaggtgcggcagcggatcggctactgcccgcagtttgatgccttgctggaccacatgacaggccgggagatgctggtcatgtacgctcggctccggggcatccctgagcgccacatcggggcctgcgtggagaacactctgcggggcctgctgctggagccacatgccaacaagctggtcaggacgtacagtggtggtaacaagcggaagctgagcaccggcatcgccctgatcggagagcctgctgtcatcttcctggacgagccgtccactggcatggaccccgtggcccggcgcctgctttgggacaccgtggcacgagcccgagagtctggcaaggccatcatcatcacctcccacagcatggaggagtgtgaggccctgtgcacccggctggccatcatggtgcaggggcagttcaagtgcctgggcagcccccagcacctcaagagcaagttcggcagcggctactccctgcgggccaaggtgcagagtgaagggcaacaggaggcgctggaggagttcaaggccttcgtggacctgacctttccaggcagcgtcctggaagatgagcaccaaggcatggtccattaccacctgccgggccgtgacctcagctgggcgaaggttttcggtattctggagaaagccaaggaaaagtacggcgtggacgactactccgtgagccagatctcgctggaacaggtcttcctgagcttcgcccacctgcagccgcccaccgcagaggaggggcgatga

SEQ ID NO: 24 - protein, 1704 aa, Homo sapiens
MAVLRQLALLLWKNYTLQKRKVLVTVLELFLPLLFSGILIWLRLKIQSENVPNATIYPGQSIQELPLFFTFPPPGDTWELAYIPSHSDAAKTVTETVRRALVINMRVRGFPSEKDFEDYIRYDNCSSSVLAAVVFEHPFNHSKEPLPLAVKYHLRFSYTRRNYMWTQTGSFFLKETEGWHTTSLFPLFPNPGPREPTSPDGGEPGYIREGFLAVQHAVDRAIMEYHADAATRQLFQRLTVTIKRFPYPPFIADPFLVAIQYQLPLLLLLSFTYTALTIARAVVQEKERRLKEYMRMMGLSSWLHWSAWFLLFFLFLLIAASFMTLLFCVKVKPNVAVLSRSDPSLVLAFLLCFAISTISFSFMVSTFFSKANMAAAFGGFLYFFTYIPYFFVAPRYNWMTLSQKLCSCLLSNVAMAMGAQLIGKFEAKGMGIQWRDLLSPVNVDDDFCFGQVLGMLLLDSVLYGLVTWYMEAVFPGQFGVPQPWYFFIMPSYWCGKPRAVAGKEEEDSDPEKALRNEYFEAEPEDLVAGIKIKHLSKVFRVGNKDRAAVRDLNLNLYEGQITVLLGHNGAGKTTTLSMLTGLFPPTSGRAYISGYEISQDMVQIRKSLGLCPQHDILFDNLTVAEHLYFYAQLKGLSRQKCPEEVKQMLHIIGLEDKWNSRSRFLSGGMRRKLSIGIALIAGSKVLILDEPTSGMDAISRRAIWDLLQRQKSDRTIVLTTHFMDEADLLGDRIAIMAKGELQCCGSSLFLKQKYGAGYHMTLVKEPHCNPEDISQLVHHHVPNATLESSAGAELSFILPRESTHRFEGLFAKLEKKQKELGIASFGASITTMEEVFLRVGKLVDSSMDIQAIQLPALQYQHERRASDWAVDSNLCGAMDPSDGIGALIEEERTAVKLNTGLALHCQQFWAMFLKKAAYSWREWKMVAAQVLVPLTCVTLALLAINYSSELFDDPMLRLTLGEYGRTVVPFSVPGTSQLGQQLSEHLKDALQAEGQEPREVLGDLEEFLIFRASVEGGGFNERCLVAASFRDVGERTVVNALFNNQAYHSPATALAVVDNLLFKLLCGPHASIVVSNFPQPRSALQAAKDQFNEGRKGFDIALNLLFAMAFLASTFSILAVSERAVQAKHVQFVSGVHVASFWLSALLWDLISFLIPSLLLLVVFKAFDVRAFTRDGHMADTLLLLLLYGWAIIPLMYLMNFFFLGAATAYTRLTIFNILSGIATFLMVTIMRIPAVKLEELSKTLDHVFLVLPNHCLGMAVSSFYENYETRRYCTSSEVAAHYCKKYNIQYQENFYAWSAPGVGRFVASMAASGCAYLILLFLIETNLLQRLRGILCALRRRRTLTELYTRMPVLPEDQDVADERTRILAPSPDSLLHTPLIIKELSKVYEQRVPLLAVDRLSLAVQKGECFGLLGFNGAGKTTTFKMLTGEESLTSGDAFVGGHRISSDVGKVRQRIGYCPQFDALLDHMTGREMLVMYARLRGIPERHIGACVENTLRGLLLEPHANKLVRTYSGGNKRKLSTGIALIGEPAVIFLDEPSTGMDPVARRLLWDTVARARESGKAIIITSHSMEECEALCTRLAIMVQGQFKCLGSPQHLKSKFGSGYSLRAKVQSEGQQEALEEFKAFVDLTFPGSVLEDEHQGMVHYHLPGRDLSWAKVFGILEKAKEKYGVDDYSVSQISLEQVFLSFAHLQPPTAEEGR

SEQ ID NO: 25 - DNA, 600 bp, Woodchuck hepatitis virus
gggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagct

SEQ ID NO: 26 - DNA, 526 bp, Artificial Sequence
  mifepristone-regulated promoter
accgagctcttacgcgggtcgaagcggagtactgtcctccgagtggagtactgtcctccgagcggagtactgtcctccgagtcgagggtcgaagcggagtactgtcctccgagtggagtactgtcctccgagcggagtactgtcctccgagtcgactctagagggtatataatggatctcgagatatcggagctcgtttagtgaaccgtcagatcgcctggagacgccatccacgctgttttgacctccatagaagacaccgggaccgatccagcctccgcggccgggaacggtgcattggaacgcgcattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccaagcttggtaccgagctcggatccactagtccagtgtggtggaattctgcagatcgaaacgatgatagatccc

SEQ ID NO: 27 - DNA, 1965 bp, Artificial Sequence
  nucleic acid sequence encoding a trans-activator for use with a mifepristone-regulated promoter
atggactcccagcagccagatctgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttagaattcccgggtgtcgaccagaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgggtcgacgcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaa